(19) United States
(12) Patent Application Publication
(10) Pub. No.: US 2023/0399331 A1
(43) Pub. Date: Dec. 14, 2023

(54) SOLID FORMS OF JAK INHIBITOR AND PROCESS OF PREPARING THE SAME

(71) Applicant: Incyte Corporation, Wilmington, DE (US)

(72) Inventors: Zhongjiang Jia, Kennett Square, PA (US); Weiguo Liu, Wilmington, DE (US); Pingli Liu, Wilmington, DE (US); David Meloni, Bear, DE (US); Yongchun Pan, Wilmington, DE (US); Jiacheng Zhou, Newark, DE (US); Travis Houston, West Lafayette, IN (US)

(21) Appl. No.: 18/209,674

(22) Filed: Jun. 14, 2023

Related U.S. Application Data
(60) Provisional application No. 63/352,094, filed on Jun. 14, 2022.
(60) Provisional application No. 63/411,808, filed on Sep. 30, 2022.

(51) Int. Cl. C07D 487/04; A61K 9/06; A61K 47/10; A61K 47/14; A61K 47/36; A61K 47/06; A61K 47/44; A61K 47/32; A61K 47/18; A61K 47/26; A61K 9/107; A61K 9/20

(52) U.S. Cl. CPC C07D 487/04; A61K 9/06; A61K 47/10; A61K 47/14; A61K 47/36; A61K 47/06; A61K 47/44; A61K 47/32; A61K 47/183; A61K 47/26; A61K 9/107; A61K 9/2054; A61K 9/2018; A61K 9/2009; A61K 9/2013; C07B 2200/13

(57) ABSTRACT

The present disclosure is related to solid forms of ruxolitinib di-hydrate and ruxolitinib free base, process of preparing the same, and compositions comprising the same.

### CROSS-REFERENCE TO RELATED APPLICATIONS

The present application claims the benefit of U.S. Provisional Application Nos. 63/352,094, filed Jun. 14, 2022, and 63/411,808, filed Sep. 30, 2022, each of which is incorporated herein by reference in its entirety.

### FIELD

The present disclosure is related to solid forms of ruxolitinib di-hydrate and ruxolitinib free base, process of preparing the same, and compositions comprising the same.

### BACKGROUND

The Janus kinase family of protein tyrosine kinases (JAKs) belong to the non-receptor type of tyrosine kinases and include family members: JAK1 (also known as Janus kinase-1), JAK2 (also known as Janus kinase-2), JAK3 (also known as Janus kinase, leukocyte; JAKL; L-JAK and Janus kinase-3) and TYK2 (also known as protein-tyrosine kinase 2). The pathway involving JAKs and Signal Transducers and Activators of Transcription (STATs) is engaged in the signaling of a wide range of cytokines. Cytokines are low-molecular weight polypeptides or glycoproteins that stimulate biological responses in virtually all cell types. Generally, cytokine receptors do not have intrinsic tyrosine kinase activity, and thus require receptor-associated kinases to propagate a phosphorylation cascade. JAKs fulfill this function. Cytokines bind to their receptors, causing receptor dimerization, and this enables JAKs to phosphorylate each other as well as specific tyrosine motifs within the cytokine receptors. STATs that recognize these phosphotyrosine motifs are recruited to the receptor, and are then themselves activated by a JAK-dependent tyrosine phosphorylation event. Upon activation, STATs dissociate from the receptors, dimerize, and translocate to the nucleus to bind to specific DNA sites and alter transcription (Scott, M. J., C. J. Godshall, et al. (2002). “Jaks, STATs, Cytokines, and Sepsis.” Clin Diagn Lab Immunol 9(6): 1153-9).

The JAK family plays a role in the cytokine-dependent regulation of proliferation and function of cells involved in immune response. The JAK/STAT pathway, and in particular all four members of the JAK family, are believed to play a role in the pathogenesis of the asthmatic response, chronic obstructive pulmonary disease, bronchitis, and other related inflammatory diseases of the lower respiratory tract. Moreover, multiple cytokines that signal through JAK kinases have been linked to inflammatory diseases or conditions of the upper respiratory tract such as those affecting the nose and sinuses (e.g., rhinitis, sinusitis) whether classically allergic reactions or not. The JAK/STAT pathway has also been implicated to play a role in inflammatory diseases/conditions of the eye including, but not limited to, iritis, uveitis, scleritis, conjunctivitis, as well as chronic allergic responses.

Consistent with the foregoing, the JAK 1/2 inhibitor ruxolitinib, ((R)-3-(4-(7H-pyrrolo[2,3-d]pyrimidin-4-yl)-1H-pyrazol-1-yl)-3-cyclopentylpropanenitrile), has been approved in the US as an oral medication for treatment of myelofibrosis, polycythemia vera, acute graft versus host disease, and chronic graft versus host disease. Ruxolitinib has also been approved as a topical cream for treatment of atopic dermatitis and has been investigated in clinical trials for the treatment of vitiligo and psoriasis.

The aforementioned approved products currently use the phosphate salt of ruxolitinib. Stable crystalline ruxolitinib phosphate is described and patented in U.S. Pat. No. 8,722,693. Given the successful use of stable crystalline ruxolitinib phosphate in approved medications, there was no need to investigate other salts or solid forms of free base. Surprisingly, however, the applicants discovered the crystalline di-hydrate form of ruxolitinib described herein during scale-up work related to manufacturing a topical product using ruxolitinib phosphate. Accordingly, crystalline ruxolitinib di-hydrate is discussed herein, along with methods of making and using crystalline ruxolitinib di-hydrate, including in certain pharmaceutical formulations.

Discussed herein are also other solid forms of ruxolitinib such as anhydrous crystalline ruxolitinib free base.

### SUMMARY

The present disclosure provides, inter alia, a solid form, which is ruxolitinib di-hydrate, having the structure below:

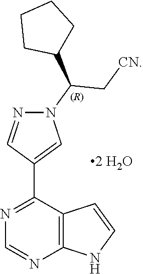

The present disclosure provides a solid form, which is crystalline ruxolitinib free base.

The present disclosure further provides processes of preparing a solid form of ruxolitinib di-hydrate comprising isolating the solid form from a solution comprising ruxolitinib free base and an aqueous solvent component. The present disclosure further provides processes of preparing anhydrous crystalline ruxolitinib free base.

The present disclosure also provides pharmaceutical compositions comprising a solid form of ruxolitinib di-hydrate. The present disclosure also provides pharmaceutical compositions comprising anhydrous crystalline ruxolitinib free base. In one aspect, the pharmaceutical composition is a topical pharmaceutical formulation. In one aspect, the pharmaceutical composition is an oral dosage form that is a sustained-release dosage form. The present disclosure further provides a process for preparing a topical pharmaceutical formulation for skin application, comprising incorporating ruxolitinib di-hydrate into the formulation. The present disclosure further provides a process for preparing a topical pharmaceutical formulation for skin application, comprising incorporating anhydrous crystalline ruxolitinib free base into the formulation.

The present disclosure further provides a process for preparing an oral formulation, comprising mixing or granulating ruxolitinib di-hydrate with one or more pharmaceutically acceptable carriers. The present disclosure further provides a process for preparing an oral formulation, comprising mixing or granulating anhydrous crystalline ruxolitinib free base with one or more pharmaceutically acceptable carriers.

The present disclosure further provides methods of treating a disease described herein in a patient in need thereof, comprising administering to the patient a pharmaceutical composition or a solid form of the disclosure.

The present disclosure further provides methods of treating a skin disorder, comprising applying a pharmaceutical composition or a solid form described herein to an affected area of skin of the patient.

The present disclosure also provides ruxolitinib di-hydrate for use in any of the methods described herein. The present disclosure also provides anhydrous crystalline ruxolitinib free base for use in any of the methods described herein. The present disclosure further provides use of ruxolitinib di-hydrate for preparation of a medicament for use in any of the methods described herein. The present disclosure further provides use of anhydrous crystalline ruxolitinib free base for preparation of a medicament for use in any of the methods described herein.

### DESCRIPTION OF DRAWINGS

FIG. 1 is an Oak Ridge Thermal Ellipsoid Plot (ORTEP) representation of the single X-ray crystal structure of crystalline ruxolitinib di-hydrate.

FIG. 2 is an X-ray powder diffraction (XRPD) pattern of one representative sample of crystalline ruxolitinib di-hydrate.

FIG. 3 is a differential scanning calorimetry (DSC) thermogram of crystalline ruxolitinib di-hydrate.

FIG. 4 is a thermogravimetric analysis (TGA) thermogram of crystalline ruxolitinib di-hydrate.

FIG. 5 is a dynamic vapor sorption (DVS) analysis of crystalline ruxolitinib di-hydrate.

FIG. 6 is a comparison of an XRPD pattern of crystalline ruxolitinib di-hydrate with an XRPD of crystalline ruxolitinib phosphate.

FIG. 7 is an XRPD pattern of crystalline ruxolitinib di-hydrate from another batch than the sample that generated the XRPD pattern in FIG. 2.

FIG. 8 is an XRPD pattern of crystalline ruxolitinib di-hydrate after DVS.

FIG. 9 is an XRPD pattern of one representative sample of crystalline ruxolitinib di-hydrate.

FIG. 10 is a DSC thermogram of one representative sample of crystalline ruxolitinib di-hydrate.

FIG. 11 is a TGA thermogram of one representative sample of crystalline ruxolitinib di-hydrate.

FIG. 12 is a DVS analysis of one representative sample of crystalline ruxolitinib di-hydrate.

FIG. 13 is an XRPD pattern of one representative sample of anhydrous crystalline ruxolitinib free base.

FIG. 14 is an XRPD pattern of anhydrous crystalline ruxolitinib free base generated via vacuum drying versus P₂O₅.

FIG. 15 is a DSC thermogram of one representative sample of anhydrous crystalline ruxolitinib free base.

FIG. 16 is a DSC thermogram of one representative sample of anhydrous crystalline ruxolitinib free base.

FIG. 17 is a TGA thermogram of one representative sample of anhydrous crystalline ruxolitinib free base.

FIG. 18 is a TGA thermogram of one representative sample of anhydrous crystalline ruxolitinib free base.

FIG. 19 is a DVS analysis of one representative sample of anhydrous crystalline ruxolitinib free base.

FIG. 20 is an XRPD pattern of anhydrous crystalline ruxolitinib free base posts DVS as compared to starting material.

FIG. 21 is a calculated XRPD pattern for ruxolitinib di-hydrate.

### DETAILED DESCRIPTION

### Ruxolitinib di-hydrate

The present disclosure provides, inter alia, a solid form which is crystalline ruxolitinib di-hydrate:

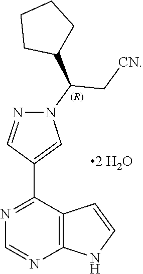

In some embodiments, the solid form is substantially isolated.

In some embodiments, the solid form is characterized by having at least one XRPD peak, in terms of 2-theta (±0.2 degrees), selected from 6.9, 10.6, 11.6, 12.9, 15.1, 15.4, 19.0, 21.8, 22.7, 23.1, 24.8, and 25.7 degrees. In some embodiments, the solid form is characterized by having at least two XRPD peaks, in terms of 2-theta (±0.2 degrees), selected from 6.9, 10.6, 11.6, 12.9, 15.1, 15.4, 19.0, 21.8, 22.7, 23.1, 24.8, and 25.7 degrees. In some embodiments, the solid form is characterized by having at least three XRPD peaks, in terms of 2-theta (±0.2 degrees), selected from 6.9, 10.6, 11.6, 12.9, 15.1, 15.4, 19.0, 21.8, 22.7, 23.1, 24.8, and 25.7 degrees. In some embodiments, the solid form is characterized by having at least four XRPD peaks, in terms of 2-theta (±0.2 degrees), selected from 6.9, 10.6, 11.6, 12.9, 15.1, 15.4, 19.0, 21.8, 22.7, 23.1, 24.8, and 25.7 degrees. In some embodiments, the solid form is characterized by having at least five XRPD peaks, in terms of 2-theta (±0.2 degrees), selected from 6.9, 10.6, 11.6, 12.9, 15.1, 15.4, 19.0, 21.8, 22.7, 23.1, 24.8, and 25.7 degrees. In some embodiments, the solid form is characterized by having at least six XRPD peaks, in terms of 2-theta (±0.2 degrees), selected from 6.9, 10.6, 11.6, 12.9, 15.1, 15.4, 19.0, 21.8, 22.7, 23.1, 24.8, and 25.7 degrees. In some embodiments, the solid form is characterized by having XRPD peaks, in terms of 2-theta (±0.2 degrees), selected from 6.9, 10.6, 11.6, 12.9, 15.1, 15.4, 19.0, 21.8, 22.7, 23.1, 24.8, and 25.7 degrees.

In some embodiments, the solid form is characterized by having XRPD peaks, in terms of 2-theta (±0.2 degrees), selected from 19.0, 22.7, and 23.1 degrees. In some embodiments, the solid form is characterized by having further XRPD peaks, in terms of 2-theta (±0.2 degrees), selected from 10.6 and 15.4 degrees. In some embodiments, the solid form is characterized by having further XRPD peaks, in terms of 2-theta (±0.2 degrees), selected from 11.6 and 25.7 degrees. In some embodiments, the solid form is characterized by having further XRPD peaks, in terms of 2-theta (±0.2 degrees), selected from 6.9 and 21.8 degrees. In some embodiments, the solid form is characterized by having further XRPD peaks, in terms of 2-theta (±0.2 degrees), selected from 12.9, 15.1, and 24.8 degrees.

In some embodiments, the solid form is characterized by having XRPD peaks, in terms of 2-theta (±0.2 degrees), selected from any one, two, three, four, five, six or more peaks recited in Tables 1, 2, 3A, or 3B. In some embodiments, the solid form is characterized by having XRPD peaks, in terms of 2-theta (±0.2 degrees), selected from any one, two, three, four, five, six or more peaks recited in Table 1. In some embodiments, the solid form is characterized by having XRPD peaks, in terms of 2-theta (±0.2 degrees), selected from any one, two, three, four, five, six or more peaks recited in Table 2. In some embodiments, the solid form is characterized by having XRPD peaks, in terms of 2-theta (±0.2 degrees), selected from any one, two, three, four, five, six or more peaks recited in Table 3A. In some embodiments, the solid form is characterized by having XRPD peaks, in terms of 2-theta (±0.2 degrees), selected from any one, two, three, four, five, six or more peaks recited in Table 3B. In some embodiments, the solid form is characterized by having XRPD peaks, in terms of 2-theta (±0.2 degrees), selected from peaks recited in Tables 1, 2, 3A, or 3B.

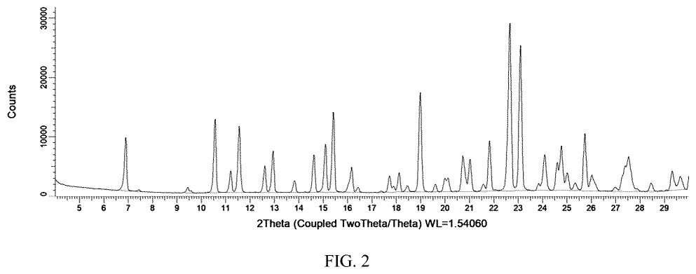

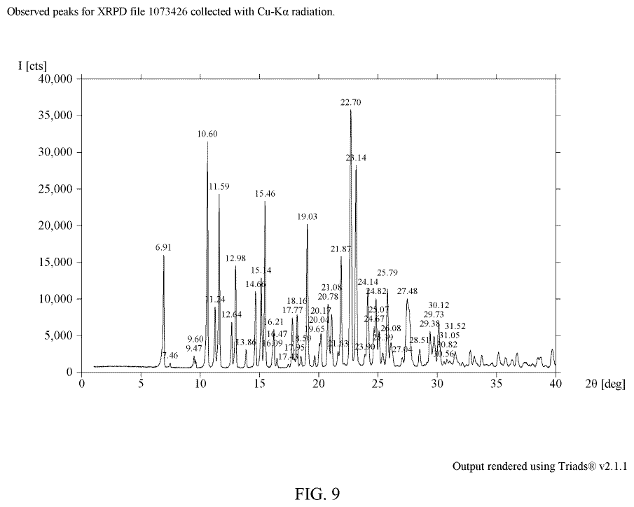

In some embodiments, the solid form is characterized by having an XRPD pattern with characteristic peaks as substantially shown in FIG. 2. In some embodiments, the solid form is characterized by having an XRPD pattern with characteristic peaks as substantially shown in FIG. 9.

In some embodiments, the solid form is characterized by single crystal x-ray diffraction having a P212121 space group and cell formula units (Z) of 8. In some embodiments, the solid form has unit cell parameters: a is about 9.97 Å, b is about 15.18 Å, c is about 23.64 Å, α is about 90°, β is about 90°, and γ is about 90°. In some embodiments, the solid form is characterized by having an endothermic peak with an onset temperature (±5° C.) at 61° C. and a maximum temperature (±5° C.) at 67° C., in a DSC thermogram. In some embodiments, the solid form is characterized by having an endothermic peak with an onset temperature (±5° C.) at 61 to 68° C. and a maximum temperature (±5° C.) at 67 to 72° C., in a DSC thermogram. In some embodiments, the solid form is characterized by having an endothermic peak with an onset temperature (±5° C.) at 68° C. and a maximum temperature (±5° C.) at 72° C., in a DSC thermogram. In some embodiments, the solid form is characterized by having a first endothermic peak with onset temperature (±5° C.) at 68° C. and a maximum temperature (±5° C.) at 72° C., and a second endothermic peak with a maximum temperature (±5° C.) at 110° C., and in a DSC thermogram.

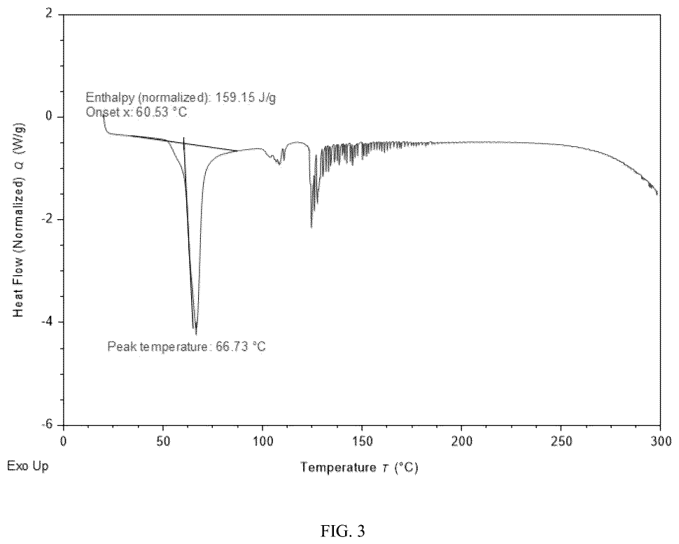

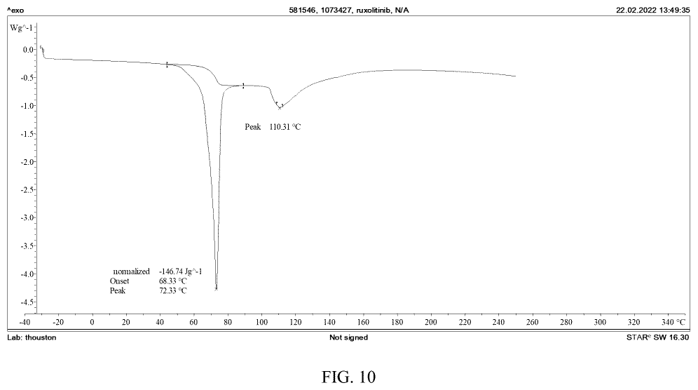

In some embodiments, the solid form is characterized by having a DSC thermogram substantially as depicted in FIG. 3. In some embodiments, the solid form is characterized by having a DSC thermogram substantially as depicted in FIG. 10.

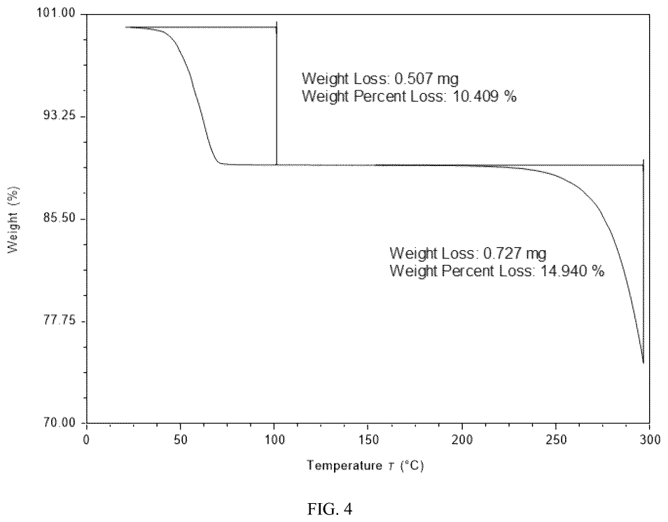

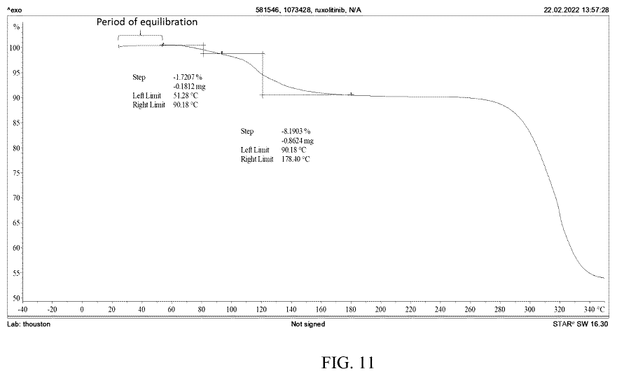

In some embodiments, the solid form is characterized by having a TGA thermogram substantially as depicted in FIG. 4. In some embodiments, the solid form is characterized by having a TGA thermogram substantially as depicted in FIG. 11.

Provided herein is a process of preparing a solid form, which is ruxolitinib di-hydrate comprising contacting ruxolitinib free base with water.

Provided herein is a process of preparing a solid form, which is ruxolitinib di-hydrate comprising isolating the solid form from a solution comprising ruxolitinib free base and an aqueous solvent component.

The isolating can comprise crystallizing the solid form from a solution comprising ruxolitinib free base and an aqueous solvent component.

In some embodiments, the crystallizing comprises cooling the solution to crystallize the solid form.

In some embodiments, the crystallizing comprises:
a) heating the solution comprising ruxolitinib free base and an aqueous solvent component; and
b) after said heating, cooling the solution to crystallize the solid form.

In some embodiments, the crystallizing comprises:
a) heating the solution comprising ruxolitinib free base and an aqueous solvent component;
b) after said heating, cooling the solution;
c) after said cooling, adding seeds of crystalline ruxolitinib di-hydrate to the solution; and
d) after said adding of said seeds, stirring the solution to crystallize the solid form.

In some embodiments, the solution is formed using amorphous ruxolitinib free base.

In step a), the solution comprising ruxolitinib free base and a solvent mixture can be heated to a temperature of from about 40° C. to about 80° C. In some embodiments, in step a), the solution comprising ruxolitinib free base and a solvent mixture is heated to a temperature of from about 50° C. to about 70° C. In some embodiments, in step a), the solution comprising ruxolitinib free base and a solvent mixture is heated to a temperature of from about 55° C. to about 65° C.

In step b), the solution can be cooled to a temperature of from about 10° C. to about 40° C. In some embodiments, in step b), the solution is cooled to a temperature of from about 15° C. to about 35° C. In some embodiments, in step b), the solution is cooled to a temperature of from about 20° C. to about 30° C. In some embodiments, in step b), the solution is cooled to a temperature of about ambient temperature.

In step d), the solution can be stirred for about 1 to about 30 hours. In some embodiments, in step d), the solution is stirred for about 10 to about 20 hours. In some embodiments, in step d), the solution is stirred for about 14 to about 18 hours.

In some embodiments, the aqueous solvent component is water.

In some embodiments, the aqueous solvent component comprises a polar protic solvent and water. In some embodiments, the polar protic solvent is an alcohol. In some embodiments, the polar protic solvent is a C₁₋₆ alcohol. In some embodiments, the C₁₋₆ alcohol is isopropanol. In some embodiments, the volume to volume ratio of the polar protic solvent to the water is about 1 to 0.1 to about 1 to 10. In some embodiments, the volume to volume ratio of polar protic solvent to water is about 1 to 0.5 to about 1 to 5. In some embodiments, the volume to volume ratio of polar protic solvent to water is about 1 to 1 to about 1 to 3. In some embodiments, the volume to volume ratio of polar protic solvent to water is about 1 to 2 to about 1 to 2.5.

The ruxolitinib free base can be prepared by a process comprising reacting ruxolitinib phosphate:

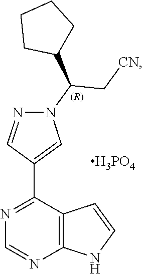

with a base in a solvent component.

In some embodiments, the ruxolitinib free base is amorphous.

In some embodiments, the reacting of ruxolitinib phosphate with a base comprises using from about 1 to about 10 molar equivalents of the base relative to ruxolitinib phosphate. In some embodiments, the reacting of ruxolitinib phosphate with a base comprises using from about 1 to about 5 molar equivalents of the base relative to ruxolitinib phosphate. In some embodiments, the reacting of ruxolitinib phosphate with a base comprises using from about 1 to about 3 molar equivalents of the base relative to ruxolitinib phosphate. In some embodiments, the reacting of ruxolitinib phosphate with a base comprises using from about 2 to about 10 molar equivalents of the base relative to ruxolitinib phosphate. In some embodiments, the reacting of ruxolitinib phosphate with a base comprises using from about 2 to about 5 molar equivalents of the base relative to ruxolitinib phosphate. In some embodiments, the reacting of ruxolitinib phosphate with a base comprises using from about 2 to about 3 molar equivalents of the base relative to ruxolitinib phosphate.

In some embodiments, the reacting of ruxolitinib phosphate with a base comprises using an amount of the base sufficient to generate a pH of about 7.5 to about 8. In some embodiments, the reacting of ruxolitinib phosphate with a base comprises using an amount of the base sufficient to generate a pH of about 7 to about 8.

In some embodiments, the base is a hydroxide base. In some embodiments, the base is an alkali metal hydroxide or an alkaline earth metal hydroxide. In some embodiments, the base is an alkali metal hydroxide. In some embodiments, the base is an alkaline earth metal hydroxide. In some embodiments, the base is KOH. In some embodiments, the base is NaOH.

In some embodiments, the solvent component comprises water. In some embodiments, the solvent component comprises one or more aprotic solvents and water. In some embodiments, the solvent component comprises water, an ester solvent, a halogenated solvent, or a mixture thereof. In some embodiments, the ester solvent is ethyl acetate. In some embodiments, the halogenated solvent is dichloromethane. In some embodiments, the solvent component comprises ethyl acetate, dichloromethane, and water.

In some embodiments, the ruxolitinib phosphate in the solvent mixture is cooled to a temperature of from about 0° C. to about 10° C. In some embodiments, the ruxolitinib phosphate in the solvent mixture is cooled to a temperature of from about 0° C. to about 5° C.

Provided herein is a solid form of ruxolitinib di-hydrate, which is prepared by any of the process described herein.

### Anhydrous Crystalline Ruxolitinib Free Base

The present disclosure also provides, inter alia, a solid form which is anhydrous crystalline ruxolitinib free base. In some embodiments, the solid form is Form I. In some embodiments, the solid form is substantially isolated.

In some embodiments, the solid form is characterized by having at least one XRPD peak, in terms of 2-theta (±0.2 degrees), selected from 7.2, 11.5, 11.6, 13.2, 14.0, 15.4, 15.7, 18.2, 19.1, 19.6, 22.0, and 23.9 degrees. In some embodiments, the solid form is characterized by having at least two XRPD peaks, in terms of 2-theta (±0.2 degrees), selected from 7.2, 11.5, 11.6, 13.2, 14.0, 15.4, 15.7, 18.2, 19.1, 19.6, 22.0, and 23.9 degrees. In some embodiments, the solid form is characterized by having at least three XRPD peaks, in terms of 2-theta (±0.2 degrees), selected from 7.2, 11.5, 11.6, 13.2, 14.0, 15.4, 15.7, 18.2, 19.1, 19.6, 22.0, and 23.9 degrees. In some embodiments, the solid form is characterized by having at least four XRPD peaks, in terms of 2-theta (±0.2 degrees), selected from 7.2, 11.5, 11.6, 13.2, 14.0, 15.4, 15.7, 18.2, 19.1, 19.6, 22.0, and 23.9 degrees. In some embodiments, the solid form is characterized by having at least five XRPD peaks, in terms of 2-theta (±0.2 degrees), selected from 7.2, 11.5, 11.6, 13.2, 14.0, 15.4, 15.7, 18.2, 19.1, 19.6, 22.0, and 23.9 degrees. In some embodiments, the solid form is characterized by having at least six XRPD peaks, in terms of 2-theta (±0.2 degrees), selected from 7.2, 11.5, 11.6, 13.2, 14.0, 15.4, 15.7, 18.2, 19.1, 19.6, 22.0, and 23.9 degrees. In some embodiments, the solid form is characterized by having XRPD peaks, in terms of 2-theta (±0.2 degrees), selected from 7.2, 11.5, 11.6, 13.2, 14.0, 15.4, 15.7, 18.2, 19.1, 19.6, 22.0, and 23.9 degrees.

In some embodiments, the solid form is characterized by having XRPD peaks, in terms of 2-theta (±0.2 degrees), selected from 7.2, 13.2, and 15.8, degrees. In some embodiments, the solid form is characterized by having further XRPD peaks, in terms of 2-theta (±0.2 degrees), selected from 19.6 and 23.9 degrees. In some embodiments, the solid form is characterized by having further XRPD peaks, in terms of 2-theta (±0.2 degrees), selected from 11.5 and 15.4 degrees. In some embodiments, the solid form is characterized by having further XRPD peaks, in terms of 2-theta (±0.2 degrees), selected from 11.6 and 19.1 degrees.

In some embodiments, the solid form is characterized by having XRPD peaks, in terms of 2-theta (±0.2 degrees), selected from any one, two, three, four, five, six or more peaks recited in Tables 9A or 9B. In some embodiments, the solid form is characterized by having XRPD peaks, in terms of 2-theta (±0.2 degrees), selected from any one, two, three, four, five, six or more peaks recited in Table 9A. In some embodiments, the solid form is characterized by having XRPD peaks, in terms of 2-theta (±0.2 degrees), selected from any one, two, three, four, five, six or more peaks recited in Table 9B. In some embodiments, the solid form is characterized by having XRPD peaks, in terms of 2-theta (±0.2 degrees), selected from peaks recited in Tables 9A or 9B.

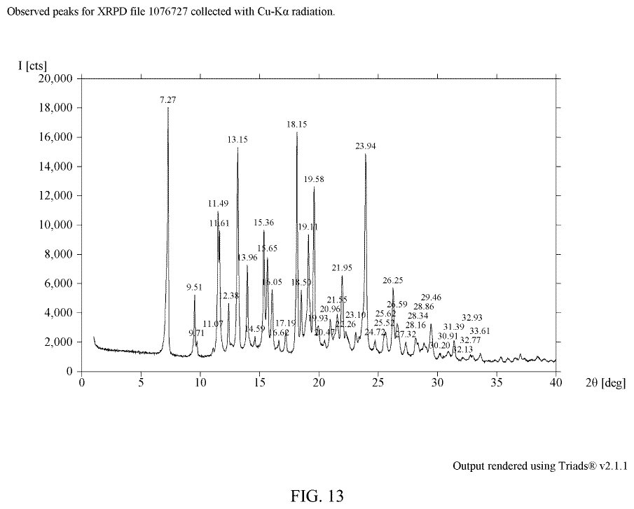

In some embodiments, the solid form is characterized by having an XRPD pattern with characteristic peaks as substantially shown in FIG. 13.

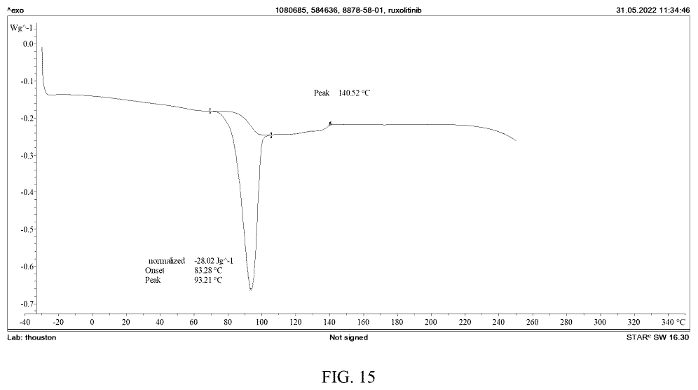

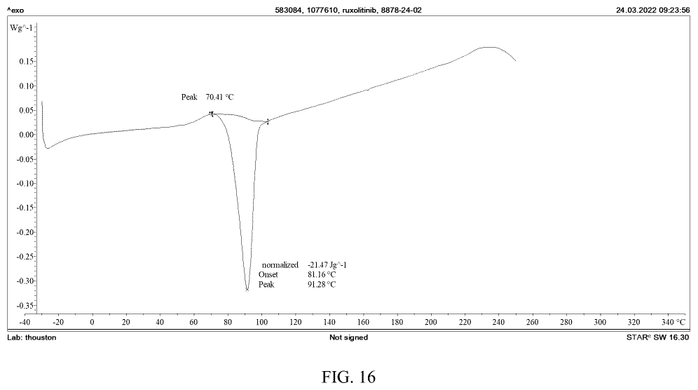

In some embodiments, the solid form is characterized by having an endothermic peak with an onset temperature (±5° C.) at 83° C. and a maximum temperature (±5° C.) at 93° C., in a DSC thermogram. In some embodiments, the solid form is characterized by having an endothermic peak with an onset temperature (±5° C.) at 81° C. and a maximum temperature (±5° C.) at 91° C., in a DSC thermogram. In some embodiments, the solid form is characterized by having a DSC thermogram substantially as depicted in FIG. 15 or FIG. 16.

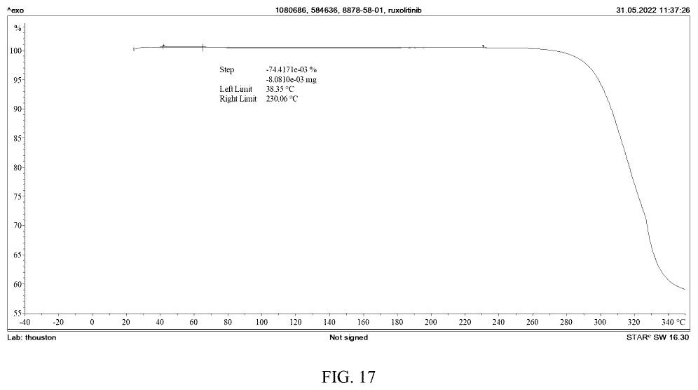

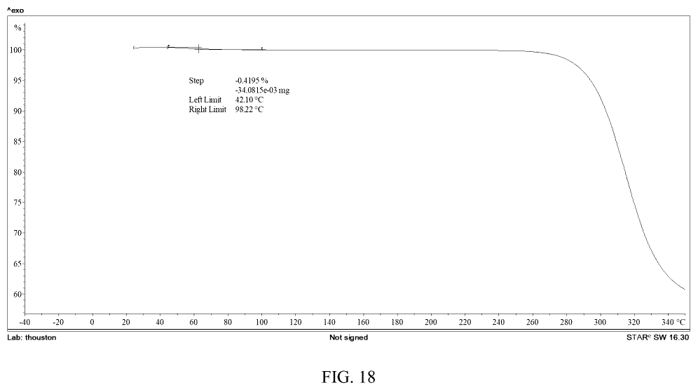

In some embodiments, the solid form is characterized by having a TGA thermogram substantially as depicted in FIG. 17 or FIG. 18.

Provided herein are processes of preparing anhydrous crystalline ruxolitinib free base. In some embodiments, the process of preparing anhydrous crystalline ruxolitinib free base comprises drying ruxolitinib di-hydrate.

The drying can include drying crystalline ruxolitinib di-hydrate in a vacuum oven from about room temperature to about 60° C. In some embodiments, the drying is carried out at about room temperature, 35° C., 40° C., 45° C., 50° C., 55° C., or 60° C. In some embodiments, the drying is carried out for about 1 day to about 10 days. In some embodiments, the drying is carried out for about 1 day to about 5 days. In some embodiments, the drying is carried out for about 1, 2, 3, 4, 5, 6, 7, 8, 9, or 10 days. In some embodiments, the drying comprising drying crystalline ruxolitinib di-hydrate in a jar with desiccant at about room temperature. In some embodiments, the desiccant is P₂O₅. In some embodiments, the drying is carried out for about 4 to 10 days. In some embodiments, the drying is carried out for about 4 to 5 days.

Provided herein is anhydrous crystalline ruxolitinib, which is prepared by any of the process described herein.

Generally, the term “about” in the context of the amounts of temperatures or equivalents of reagents using in the synthetic process described herein means±10%. In some embodiments, the term “about” means±5%.

Solid forms can be detected, identified, and characterized by well-known techniques, such as, but not limited to, X-ray powder diffraction (XRPD). Other characterization methods such as differential scanning calorimetry (DSC), thermogravimetric analysis (TGA), dynamic vapor sorption (DVS), single crystal X-ray diffractometry, vibrational spectroscopy, solution calorimetry, solid state nuclear magnetic resonance (NMR), infrared (IR) spectroscopy, Raman spectroscopy, hot stage optical microscopy, scanning electron microscopy (SEM), electron crystallography and quantitative analysis, particle size analysis (PSA), surface area analysis, solubility, and rate of dissolution further help identify the form as well as help determine stability and solvent/water content.

An XRPD pattern of reflections (peaks) is typically considered to be representative of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, “peak” refers to any peak or other special feature that one skilled in the art would recognize as not attributable to background noise. Peak assignments, such as those reported herein, can vary by plus or minus about 0.2° (2-theta). The terms “substantially” and “about” as used in the context of XRPD herein are meant to encompass all of the above-mentioned variations.

In the same way, temperature readings in connection with DSC, TGA, or other thermal experiments can vary about ±5° C. depending on the instrument, particular settings, sample preparation, etc. Accordingly, a crystalline form reported herein having a DSC thermogram “substantially” as shown in any of the Figures or the term “about” is understood to accommodate such variation.

In some embodiments, the solid forms described herein can be substantially isolated. By “substantially isolated” is meant that the solid form is at least partially or substantially separated from the environment in which it was formed or detected. Partial separation can include, for example, a composition enriched with the compound or the intermediate. Substantially isolated can include compositions containing at least about 50%, at least about 60%, at least about 70%, at least about 80%, at least about 90%, at least about 95%, at least about 97%, or at least about 99% by weight of the compound or intermediate.

In some embodiments, ruxolitinib di-hydrate is crystalline.

The expressions “ambient temperature,” “room temperature,” and “rt,” as used herein, are understood in the art and refer generally to a temperature, e.g., a reaction temperature, that is about the temperature of the room in which the reaction is carried out, for example, a temperature from about 20° C. to about 30° C.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, or spectrophotometry (e.g., UV-visible); or by chromatography such as high performance liquid chromatography (HPLC) or thin layer chromatography (TLC) or other related techniques.

As used herein, the terms “reacting” and “contacting” are used as known in the art and generally refers to the bringing together of chemical reagents in such a manner so as to allow their interaction at the molecular level to achieve a chemical or physical transformation. In some embodiments, the reacting involves two reagents, wherein one or more equivalents of second reagent are used with respect to the first reagent. The reacting steps of the processes described herein can be conducted for a time and under conditions suitable for preparing the identified product.

The reactions of the processes described herein can be carried out in suitable solvents. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected. In some embodiments, reactions can be carried out in the absence of solvent, such as when at least one of the reagents is a liquid or gas.

Suitable solvents can include halogenated solvents such as carbon tetrachloride, bromodichloromethane, dibromochloromethane, bromoform, chloroform, bromochloromethane, dibromomethane, butyl chloride, dichloromethane, tetrachloroethylene, trichloroethylene, 1,1,1-trichloroethane, 1,1,2-trichloroethane, 1,1-di chloroethane, 2-chloropropane, 1,1,1-trifluorotoluene, 1,2-dichloroethane, 1,2-dibromoethane, hexafluorobenzene, 1,2,4-trichlorobenzene, 1,2-dichlorobenzene, chlorobenzene, fluorobenzene, mixtures thereof and the like.

Suitable solvents can include ether solvents such as: dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, t-butyl methyl ether, mixtures thereof and the like.

Suitable protic solvents can include, by way of example and without limitation, water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, mixtures thereof, and the like.

Suitable aprotic solvents can include, by way of example and without limitation, tetrahydrofuran (TRF), N,N-dimethylformamide (DMF), N,N-dimethylacetamide (DMA), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, hexamethylphosphoramide, mixtures thereof, and the like.

Suitable hydrocarbon solvents include benzene, cyclohexane, pentane, hexane, toluene, cycloheptane, methylcyclohexane, heptane (e.g., n-heptane), ethylbenzene, m-, o-, or p-xylene, octane, indane, nonane, naphthalene, mixtures thereof, and the like.

The reactions of the processes described herein can be carried out at appropriate temperatures. Reaction temperatures will depend on, for example, the melting and boiling points of the reagents and solvent, if present; thermodynamics of the reaction (e.g., vigorously exothermic reactions may need to be carried out at reduced temperatures); and the kinetics of the reaction (e.g., a high activation energy barrier may need elevated temperatures). “Elevated temperature” refers to temperatures above room temperature (room temperature can include a temperature from about 20° C. to about 30° C.).

The reactions of the processes described herein can be carried out in air or under an inert atmosphere. Typically, reactions containing reagents or products that are substantially reactive with air can be carried out using air-sensitive synthetic techniques that are well known to the skilled artisan.

Example bases include alkali metal hydroxides (e.g., lithium hydroxide, sodium hydroxide, and potassium hydroxide), alkaline earth metal hydroxide (e.g., calcium hydroxide), and alkali metal carbonate (e.g., lithium carbonate, sodium carbonate, and potassium carbonate). Some examples of strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include sodium and potassium salts of methyl, ethyl, n-propyl, i-propyl, n-butyl, t-butyl, trimethylsilyl and cyclohexyl substituted amides.

Upon carrying out preparation of the solid forms of the disclosure (and intermediates for making the solid forms), concentration, filtration, extraction, solid-phase extraction, recrystallization, chromatography, and the like may be used to isolate the desired product(s).

### Pharmaceutical Formulations and Dosage Forms

Generally, the term “about” in the context of the amounts of excipient or active ingredients in the compositions, formulations, and dosage forms described herein means ±10%. In some embodiments, the term “about” means±5%.

When employed as pharmaceuticals, the compounds or solid forms described herein can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Also provided herein is a pharmaceutical composition comprising a solid form described herein. The pharmaceutical compositions contain, as the active ingredient, one or more of the solid forms described herein in combination with one or more pharmaceutically acceptable carriers (excipients). In making the compositions, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions can be formulated so as to provide quick, sustained or delayed release of the active ingredient after administration to the patient.

As used herein, “a free base basis” means that the weight of ruxolitinib is calculated based on the weight of ruxolitinib free base in the total composition or dosage form. For example, 10 mg of ruxolitinib di-hydrate on a free base basis means 11.18 mg of ruxolitinib di-hydrate, which equates to 10 mg of ruxolitinib free base.

In some embodiments, the composition comprises from about 5 mg to about 50 mg of ruxolitinib di-hydrate on a free base basis. In some embodiments, the composition comprises from about 5 mg to about 25 mg of ruxolitinib di-hydrate on a free base basis. In some embodiments, the composition comprises from about 10 mg to about 50 mg of ruxolitinib di-hydrate on a free base basis. In some embodiments, the composition comprises from about 10 mg to about 40 mg of ruxolitinib di-hydrate on a free base basis. In some embodiments, the composition comprises from about 10 mg to about 30 mg of ruxolitinib di-hydrate on a free base basis. In some embodiments, the composition comprises about 5 mg of ruxolitinib di-hydrate on a free base basis. In some embodiments, the composition comprises about 10 mg of ruxolitinib di-hydrate on a free base basis. In some embodiments, the composition comprises about 15 mg of ruxolitinib di-hydrate on a free base basis. In some embodiments, the composition comprises about 20 mg of ruxolitinib di-hydrate on a free base basis. In some embodiments, the composition comprises about 25 mg of ruxolitinib di-hydrate on a free base basis. In some embodiments, the composition comprises about 30 mg of ruxolitinib di-hydrate on a free base basis. In some embodiments, the composition comprises about 40 mg of ruxolitinib di-hydrate on a free base basis. In some embodiments, the composition comprises about 50 mg of ruxolitinib di-hydrate on a free base basis. In some embodiments, each of the foregoing compositions is an oral dosage form. In some embodiments, each of the foregoing compositions is a sustained-release oral dosage form. In some embodiments, each of the foregoing dosage forms is a tablet. In some embodiments, each of the foregoing dosage forms is a capsule. In some embodiments, each of the foregoing compositions is a topical formulation.

In some embodiments, the composition comprises from about 5 mg to about 50 mg of anhydrous crystalline ruxolitinib free base. In some embodiments, the composition comprises from about 5 mg to about 25 mg of anhydrous crystalline ruxolitinib free base. In some embodiments, the composition comprises from about 10 mg to about 50 mg of anhydrous crystalline ruxolitinib free base. In some embodiments, the composition comprises from about mg to about 40 mg of anhydrous crystalline ruxolitinib free base. In some embodiments, the composition comprises from about 10 mg to about 30 mg of anhydrous crystalline ruxolitinib free base. In some embodiments, the composition comprises about 5 mg of anhydrous crystalline ruxolitinib free base. In some embodiments, the composition comprises about 10 mg of anhydrous crystalline ruxolitinib free base. In some embodiments, the composition comprises about 15 mg of anhydrous crystalline ruxolitinib free base. In some embodiments, the composition comprises about 20 mg of anhydrous crystalline ruxolitinib free base. In some embodiments, the composition comprises about 25 mg of anhydrous crystalline ruxolitinib free base. In some embodiments, the composition comprises about 30 mg of anhydrous crystalline ruxolitinib free base. In some embodiments, the composition comprises about 40 mg of anhydrous crystalline ruxolitinib free base. In some embodiments, the composition comprises about 50 mg of anhydrous crystalline ruxolitinib free base. In some embodiments, each of the foregoing compositions is an oral dosage form. In some embodiments, each of the foregoing compositions is a sustained-release oral dosage form. In some embodiments, each of the foregoing dosage forms is a tablet. In some embodiments, each of the foregoing dosage forms is a capsule. In some embodiments, each of the foregoing compositions is a topical formulation.

In some embodiments, the present disclosure provides an oral dosage form comprising ruxolitinib di-hydrate. In some embodiments, the oral dosage form comprises ruxolitinib di-hydrate, which is present in an amount of less than about 0.9%, less than about 0.8%, less than about 0.7%, less than about 0.6%, less than about 0.5%, less than about 0.4%, less than about less than about 0.2%, less than about 0.1%, less than about 0.09%, less than about 0.08%, less than about 0.07%, less than about 0.06%, less than about 0.05%, less than about 0.04%, less than about 0.008%, less than about 0.007%, less than about 0.006%, less than about 0.005%, less than about 0.004%, less than about 0.003%, less than about 0.002%, or less than about 0.001% by weight of the total ruxolitinib on a free base basis in the dosage form. In some embodiments, the oral dosage form comprises ruxolitinib di-hydrate, which is present in an amount of less than about 0.9%, less than about 0.8%, less than about 0.7%, less than about 0.6%, less than about 0.5%, less than about 0.4%, less than about 0.3%, less than about 0.2%, less than about 0.1%, less than about 0.09%, less than about 0.08%, less than about 0.07%, less than about 0.06%, less than about 0.05%, less than about 0.04%, less than about 0.03%, less than about 0.02%, less than about 0.01%, less than about 0.009%, less than about 0.008%, less than about 0.007%, less than about 0.006%, less than about 0.005%, less than about 0.004%, less than about 0.003%, less than about 0.002%, or less than about 0.001% by weight of the dosage form.

For preparing solid compositions such as tablets, the principal active ingredient is mixed with a pharmaceutical excipient to form a solid preformulation composition containing a homogeneous mixture of the active ingredient. When referring to these preformulation compositions as homogeneous, the active ingredient is typically dispersed evenly throughout the composition so that the composition can be readily subdivided into equally effective unit dosage forms such as tablets, pills and capsules.

The tablets or pills can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions described herein can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by topical administration. In some embodiments, the compositions are administered by topical administration to the skin. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the excipients, carriers, or stabilizers described herein will result in the formation of pharmaceutical salts.

The therapeutic dosage of the solid forms described herein can vary according to, for example, the particular use for which the treatment is made, the manner of administration of the compound, the health and condition of the patient, and the judgment of the prescribing physician. The proportion or concentration of the solid forms in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

In some embodiments, the pharmaceutical composition is an oral dosage form.

In some embodiments, the oral dosage form is an immediate dosage form. In some embodiments, the ruxolitinib di-hydrate is present in an amount of about 5 to about 25 mg on a free base basis. In some embodiments, the ruxolitinib di-hydrate is present in an amount of about mg, about 10 mg, about 15 mg, about 20 mg, or about 25 mg of ruxolitinib di-hydrate on a free base basis. In some embodiments, the anhydrous crystalline ruxolitinib free base is present in an amount of about 5 to about 25 mg. In some embodiments, the anhydrous crystalline ruxolitinib free base is present in an amount of about 5 mg, about 10 mg, about 15 mg, about 20 mg, or about 25 mg.

### Sustained-Release Dosage Forms

In some embodiments, the pharmaceutical composition is an oral dosage form.

In some embodiments, the oral dosage form is a sustained-release dosage form comprising a solid form described herein.

In some embodiments, the sustained release form comprises ruxolitinib di-hydrate as an active ingredient. In some embodiments, the ruxolitinib di-hydrate is present in an amount of about 10 to about 50 mg on a free base basis. In some embodiments, the ruxolitinib di-hydrate is present in an amount of about 10 mg, about 12.5 mg, about 15 mg, about 20 mg, about 25 mg, about 30 mg, about 35 mg, about 37.5 mg, about 40 mg, about 45 mg, or about 50 mg of on a free base basis. In some embodiments, the ruxolitinib di-hydrate is present in an amount of about mg, about 20 mg, about 30 mg, about 40 mg, or about 50 mg of ruxolitinib di-hydrate on a free base basis.

In some embodiments, the sustained release form comprises anhydrous crystalline ruxolitinib free base as an active ingredient. In some embodiments, the anhydrous crystalline ruxolitinib free base is present in an amount of about 10 to about 50 mg. In some embodiments, the anhydrous crystalline ruxolitinib free base is present in an amount of about 10 mg, about 12.5 mg, about 15 mg, about 20 mg, about 25 mg, about 30 mg, about 35 mg, about 37.5 mg, about 40 mg, about 45 mg, or about 50 mg of In some embodiments, the anhydrous crystalline ruxolitinib free base is present in an amount of about 10 mg, about 20 mg, about 30 mg, about 40 mg, or about 50 mg.

In some embodiments, the oral, sustained-release dosage form comprises ruxolitinib di-hydrate, which is present in an amount of less than about 0.9%, less than about 0.8%, less than about 0.7%, less than about 0.6%, less than about 0.5%, less than about 0.4%, less than about less than about 0.2%, less than about 0.1%, less than about 0.09%, less than about 0.08%, less than about 0.07%, less than about 0.06%, less than about 0.05%, less than about 0.04%, less than about 0.03%, less than about 0.02%, less than about 0.01%, less than about 0.009%, less than about 0.008%, less than about 0.007%, less than about 0.006%, less than about 0.005%, less than about 0.004%, less than about 0.003%, less than about 0.002%, or less than about 0.001% by weight of the total ruxolitinib on a free base basis in the dosage form. In some embodiments, the oral, sustained-release dosage form comprises ruxolitinib di-hydrate, which is present in an amount of less than about 0.9%, less than about 0.8%, less than about 0.7%, less than about less than about 0.5%, less than about 0.4%, less than about 0.3%, less than about 0.2%, less than about 0.1%, less than about 0.09%, less than about 0.08%, less than about 0.07%, less than about 0.06%, less than about 0.05%, less than about 0.04%, less than about 0.03%, less than about 0.02%, less than about 0.01%, less than about 0.009%, less than about 0.008%, less than about 0.007%, less than about 0.006%, less than about 0.005%, less than about 0.004%, less than about 0.003%, less than about 0.002%, or less than about 0.001% by weight of the dosage form.

The dosage forms can comprise a sustained-release matrix former. In some embodiments, the dosage form comprises about 10% to about 30% by weight of a sustained-release matrix former. In some embodiments, the sustained-release matrix former is one or more cellulosic ethers. In some embodiments, the sustained-release matrix former is hydroxypropyl methylcellulose (HPMC, hypromellose) (which is a high viscosity polymer). In some embodiments, the dosage form comprises about 10% to about 30% by weight of one or more hydroxypropyl methylcellulose(s). In some embodiments, the formulation has about 20% by weight of one or more hydroxypropyl methylcelluloses. Example hydroxypropyl methylcelluloses include Methocel K15M, Methocel K4M, and Methocel K100LV.

The sustained-release dosage forms described herein can further include one or more fillers, glidants, disintegrants, binders, or lubricants as inactive ingredients. Fillers can be present in the formulations in an amount of 0% to about 85% by weight. In some embodiments, the formulation has about 50% to about 80%, about 55% to about 75%, or about 60% to about 70% by weight of filler. Non-limiting examples of fillers include lactose monohydrate, microcrystalline cellulose, starch 1500, and lactose anhydrous, or combinations thereof. In some embodiments, the filler comprises microcrystalline cellulose, lactose monohydrate, or both.

Lubricants can be present in the dosage forms described herein in an amount of 0% to about 5% by weight. Non-limiting examples of lubricants include magnesium stearate, stearic acid (stearin), hydrogenated oil, polyethylene glycol, sodium stearyl fumarate, and glyceryl behenate. In some embodiments, the formulations include magnesium stearate, stearic acid, or both.

Glidants can be present in the dosage forms described herein in an amount of 0 to about 5% by weight. Non-limiting examples of glidants include talc, colloidal silicon dioxide, and cornstarch. In some embodiments, the glidant is colloidal silicon dioxide.

Disintegrants can be present in the dosage forms described herein in an amount of 0% to about 10% by weight. Non-limiting examples of disintegrants include croscarmellose sodium, crospovidone, starch, cellulose, and low substituted hydroxypropyl cellulose. Croscarmellose sodium is a preferred disintegrant.

Film-coating agents can be present in an amount of 0% to about 5% by weight. Non-limiting illustrative examples of film-coating agents include hypromellose or polyvinyl alcohol based coating with titanium dioxide, talc and optionally colorants available in several commercially available complete coating systems.

Examples of sustained release dosage forms include tablets, caplets, capsules, and the like, containing any of the sustained-release formulations described herein. Dosage forms can further include pharmaceutically acceptable coatings, pigments, or dyes.

The dosage forms contain a sustained-release formulation that results in the relatively slow release of ruxolitinib once administered, characterized by particular pharmacokinetic parameters different from those of an immediate-release formulation. The sustained-release dosage forms can minimize potentially harmful spikes in drug plasma concentrations that are associated with immediate-release formulations, and can help provide continuous, steady, and therapeutically effective plasma levels of drug. The dosage forms can be administered to a human patient as needed for therapeutic efficacy against the disease being treated, for example, once daily.

### Topical and Cream Formulations

In some embodiments, the pharmaceutical composition is a topical pharmaceutical formulation. In some embodiments described herein, the topical pharmaceutical formulation is suitable for skin application.

In some embodiments, the topical formulation comprises from about 0.5% to about 1.5% of ruxolitinib di-hydrate on a free base basis by weight of the topical formulation. In some embodiments, the topical formulation comprises about 1.5% of ruxolitinib di-hydrate on a free base basis by weight of the topical formulation. In some embodiments, the topical formulation comprises about 1.0% of ruxolitinib di-hydrate on a free base basis by weight of the topical formulation. In some embodiments, the topical formulation comprises about 0.75% of ruxolitinib di-hydrate on a free base basis by weight of the topical formulation. In some embodiments, the topical formulation comprises about 0.5% of ruxolitinib di-hydrate on a free base basis by weight of the topical formulation.

In some embodiments, the topical formulation comprises from about 0.5% to about 1.5% of anhydrous crystalline ruxolitinib free base by weight of the topical formulation. In some embodiments, the topical formulation comprises about 1.5% of anhydrous crystalline ruxolitinib free base by weight of the topical formulation. In some embodiments, the topical formulation comprises about 1.0% of anhydrous crystalline ruxolitinib free base by weight of the topical formulation. In some embodiments, the topical formulation comprises about 0.75% of anhydrous crystalline ruxolitinib free base by weight of the topical formulation. In some embodiments, the topical formulation comprises about 0.5% of anhydrous crystalline ruxolitinib free base by weight of the topical formulation.

In some embodiments, the topical formulation is prepared by dissolving ruxolitinib di-hydrate in a solvent component. In some embodiments, the solvent component comprises water.

When ruxolitinib di-hydrate is completely dissolved in a solvent component, it is generally understood to be present in the formulation as ruxolitinib free base, the crystal structure being lost upon dissolution.

In some embodiments, the topical formulation is prepared by dissolving anhydrous crystalline ruxolitinib free base in a solvent component. In some embodiments, the solvent component comprises water. When anhydrous crystalline ruxolitinib free base is completely dissolved in a solvent component, it is generally understood to be present in the formulation as ruxolitinib free base, the crystal structure being lost upon dissolution.

In some embodiments, the topical formulation comprises from about 0.5% to about 1.5% of ruxolitinib on a free base basis by weight of the topical formulation. In some embodiments, the topical formulation comprises about 1.5% of ruxolitinib on a free base basis by weight of the topical formulation. In some embodiments, the topical formulation comprises about 1.0% of ruxolitinib on a free base basis by weight of the topical formulation. In some embodiments, the topical formulation comprises about 0.75% of ruxolitinib on a free base basis by weight of the topical formulation. In some embodiments, the topical formulation comprises about 0.5% of ruxolitinib on a free base basis by weight of the topical formulation.

In some embodiments, the topical formulation comprises ruxolitinib di-hydrate, which is present in an amount of less than about 0.9%, less than about 0.8%, less than about 0.7%, less than about 0.6%, less than about 0.5%, less than about 0.4%, less than about 0.3%, less than about 0.2%, less than about 0.1%, less than about 0.09%, less than about 0.08%, less than about less than about 0.06%, less than about 0.05%, less than about 0.04%, less than about less than about 0.02%, less than about 0.01%, less than about 0.009%, less than about less than about 0.007%, less than about 0.006%, less than about 0.005%, less than about less than about 0.003%, less than about 0.002%, or less than about 0.001% on a free base basis in the formulation.

In some embodiments, the topical formulation comprises ruxolitinib di-hydrate, which is present in an amount of less than about 0.9%, less than about 0.8%, less than about 0.7%, less than about 0.6%, less than about 0.5%, less than about 0.4%, less than about 0.3%, less than about 0.2%, less than about 0.1%, less than about 0.09%, less than about 0.08%, less than about 0.07%, less than about 0.06%, less than about 0.05%, less than about 0.04%, less than about less than about 0.02%, less than about 0.01%, less than about 0.009%, less than about less than about 0.007%, less than about 0.006%, less than about 0.005%, less than about less than about 0.003%, less than about 0.002%, or less than about 0.001% on a free base basis by weight of the formulation.

In some embodiments, the topical formulation comprises (a) ruxolitinib free base in an amount of about 0.5% to about 1.5% by weight of the formulation; and (b) ruxolitinib di-hydrate, which is present in an amount of less than about 0.9%, less than about 0.8%, less than about less than about 0.6%, less than about 0.5%, less than about 0.4%, less than about 0.3%, less than about 0.2%, less than about 0.1%, less than about 0.09%, less than about 0.08%, less than about 0.07%, less than about 0.06%, less than about 0.05%, less than about 0.04%, less than about 0.03%, less than about 0.02%, less than about 0.01%, less than about 0.009%, less than about 0.008%, less than about 0.007%, less than about 0.006%, less than about 0.005%, less than about 0.004%, less than about 0.003%, less than about 0.002%, or less than about 0.001% on a free base basis by weight of the formulation.

In some embodiments, the topical pharmaceutical formulation is prepared at a large batch size. In some embodiments, the topical pharmaceutical formulation is prepared at a batch size of 1000 kg or higher.

In some embodiments, the present disclosure provides a topical pharmaceutical formulation, comprising (a) ruxolitinib, or a pharmaceutically acceptable salt thereof, and (b) ruxolitinib di-hydrate, which is present in an amount of less than about 0.9%, less than about 0.8%, less than about 0.7%, less than about 0.6%, less than about 0.5%, less than about 0.4%, less than about 0.3%, less than about 0.2%, less than about 0.1%, less than about 0.09%, less than about 0.08%, less than about 0.07%, less than about 0.06%, less than about 0.05%, less than about 0.04%, less than about 0.03%, less than about 0.02%, less than about 0.01%, less than about 0.009%, less than about 0.008%, less than about 0.007%, less than about 0.006%, less than about 0.005%, less than about 0.004%, less than about 0.003%, less than about 0.002%, or less than about 0.001% on a free base basis by weight of the formulation. In some embodiments, the ruxolitinib, or the pharmaceutically acceptable salt thereof, is present in an amount of about to about 1.5% on a free base basis by weight of the formulation. In some embodiments, the ruxolitinib, or the pharmaceutically acceptable salt thereof, is present in an amount of about 1.5% on a free base basis by weight of the formulation. In some embodiments, the ruxolitinib, or the pharmaceutically acceptable salt thereof, is ruxolitinib phosphate. In some embodiments, the formulation is prepared at a large batch size. In some embodiments, the batch size is 1000 kg or higher.

In some embodiments, the present disclosure provides a topical pharmaceutical formulation, comprising ruxolitinib, or a pharmaceutically acceptable salt thereof, wherein the formulation comprises less than about 0.9%, less than about 0.8%, less than about 0.7%, less than about 0.6%, less than about 0.5%, less than about 0.4%, less than about 0.3%, less than about 0.2%, less than about 0.1%, less than about 0.09%, less than about 0.08%, less than about less than about 0.06%, less than about 0.05%, less than about 0.04%, less than about less than about 0.02%, less than about 0.01%, less than about 0.009%, less than about 0.008%, less than about 0.007%, less than about 0.006%, less than about 0.005%, less than about less than about 0.003%, less than about 0.002%, or less than about 0.001% of ruxolitinib di-hydrate on a free basis basis by weight of the formulation, wherein the formulation is prepared at a large batch size. In some embodiments, the ruxolitinib, or the pharmaceutically acceptable salt thereof, is present in an amount of about 0.5% to about 1.5% on a free base basis by weight of the formulation. In some embodiments, the ruxolitinib, or the pharmaceutically acceptable salt thereof, is present in an amount of about 1.5% on a free base basis by weight of the formulation. In some embodiments, the ruxolitinib, or the pharmaceutically acceptable salt thereof, is ruxolitinib phosphate. In some embodiments, the batch size is 1000 kg or higher.

In some embodiments, provided herein is a process for releasing a batch of the topical pharmaceutical formulation described herein, comprising (i) testing a sample of the topical pharmaceutical formulation for the absence of crystalline ruxolitinib di-hydrate; and, if the sample passes the test in step (i), then: (ii) releasing the batch for public use. In some embodiments, the testing comprises observing a sample of the formulation under a light microscope in order to detect the absence or presence of crystals. In some embodiments, the testing comprises observing a sample of the formulation under a light microscope in order to detect the absence or presence of crystals, wherein the sample passes the test when crystals are not detected.

In some embodiments, the topical formulation is a cream formulation. In some embodiments, the cream formulation is an oil-in-water emulsion.

In some embodiments, the topical formulation is prepared by incorporating ruxolitinib di-hydrate in the formulation. In some embodiments, the topical formulation is prepared by dissolving ruxolitinib di-hydrate in a solvent component.

In some embodiments, the cream formulation is prepared by incorporating ruxolitinib di-hydrate in an oil-in-water emulsion.

In some embodiments, the emulsion comprises from about 0.5% to about 1.5% of ruxolitinib di-hydrate on a free base basis by weight of the emulsion. In some embodiments, the emulsion comprises about 1.5% of ruxolitinib di-hydrate on a free base basis by weight of the emulsion. In some embodiments, the emulsion comprises about 1.0% of ruxolitinib di-hydrate on a free base basis by weight of the emulsion. In some embodiments, the emulsion comprises about of ruxolitinib di-hydrate on a free base basis by weight of the emulsion. In some embodiments, the emulsion comprises about 0.5% of ruxolitinib di-hydrate on a free base basis by weight of the emulsion.

In some embodiments, the emulsion comprises from about 0.5% to about 1.5% of ruxolitinib on a free base basis by weight of the emulsion. In some embodiments, the emulsion comprises about 1.5% of ruxolitinib on a free base basis by weight of the emulsion. In some embodiments, the emulsion comprises about 1.0% of ruxolitinib on a free base basis by weight of the emulsion. In some embodiments, the emulsion comprises about 0.75% of ruxolitinib on a free base basis by weight of the emulsion. In some embodiments, the emulsion comprises about 0.5% of ruxolitinib on a free base basis by weight of the emulsion.

As used herein, “% ruxolitinib on a free base basis” or “on a free base basis” in the context of emulsions or topical formulations prepared by incorporating ruxolitinib di-hydrate in the emulsion means that the % w/w is calculated based on the weight of ruxolitinib free base in the total emulsion. For example, for 100 grams of a 1.5% (w/w) formulations, 1.68 grams of ruxolitinib di-hydrate would need to be incorporated in the emulsion or topical formulation to give 1.5 grams of ruxolitinib free base.

In some embodiments, the cream is a solubilized cream.

In some embodiments, the cream has a pH from about 2.8 to about 3.6. In the context of pH, “about” refers to ±0.5 (preferably ±0.3 or more preferably ±0.2).

In some embodiments, the cream formulation is any of the cream formulations disclosed in U.S. Patent Publ. No. 2015/0250790, except using ruxolitinib di-hydrate or anhydrous crystalline ruxolitinib free base as described herein, which is incorporated herein by reference in its entirety.

In some embodiments, the cream is an oil-in-water emulsion as described in US 2015/0250790, which is incorporated herein by reference in its entirety. In particular, Examples 3-6 of US 2015/0250790 (and particularly Tables 3-5 and accompanying text) are incorporated herein by reference.

In some embodiments, the oil component is present in an amount of about 10% to about 40% by weight of the emulsion.

In some embodiments, the oil component is present in an amount of about 10% to about 24% by weight of the emulsion.

In some embodiments, the oil component is present in an amount of about 15% to about 24% by weight of the emulsion.

In some embodiments, the oil component is present in an amount of about 18% to about 24% by weight of the emulsion.

In some embodiments, the oil component comprises one or more substances independently selected from petrolatums, fatty alcohols, mineral oils, triglycerides, and silicone oils.

In some embodiments, the oil component comprises one or more substances independently selected from white petrolatum, cetyl alcohol, stearyl alcohol, light mineral oil, medium chain triglycerides, and dimethicone.

In some embodiments, the oil component comprises an occlusive agent component.

In some embodiments, the occlusive agent component is present in an amount of about 2% to about 15% by weight of the emulsion.

In some embodiments, the occlusive agent component is present in an amount of about 5% to about 10% by weight of the emulsion.

In some embodiments, the occlusive agent component comprises one or more substances selected from fatty acids (e.g., lanolin acid), fatty alcohols (e.g., lanolin alcohol), hydrocarbon oils & waxes (e.g., petrolatum), polyhydric alcohols (e.g., propylene glycol), silicones (e.g., dimethicone), sterols (e.g., cholesterol), vegetable or animal fat (e.g., cocoa butter), vegetable wax (e.g., Carnauba wax), and wax ester (e.g., bees wax).

In some embodiments, the occlusive agent component comprises one or more substances selected from lanolin acid fatty alcohols, lanolin alcohol, petrolatum, propylene glycol, dimethicone, cholesterol, cocoa butter, Carnauba wax, and bees wax.

In some embodiments, the occlusive agent component comprises petrolatum.

In some embodiments, the occlusive agent component comprises white petrolatum.

In some embodiments, the oil component comprises a stiffening agent component.

In some embodiments, the stiffening agent component is present in an amount of about 2% to about 8% by weight of the emulsion.

In some embodiments, the stiffening agent component is present in an amount of about 3% to about 6% by weight of the emulsion.

In some embodiments, the stiffening agent component is present in an amount of about 4% to about 7% by weight of the emulsion.

In some embodiments, the stiffening agent component comprises one or more substances independently selected from fatty alcohols.

In some embodiments, the stiffening agent component comprises one or more substances independently selected from C₁₂₋₂₀ fatty alcohols.

In some embodiments, the stiffening agent component comprises one or more substances independently selected from C₁₆₋₁₈ fatty alcohols.

In some embodiments, the stiffening agent component comprises one or more substances independently selected from cetyl alcohol and stearyl alcohol.

In some embodiments, the oil component comprises an emollient component.

In some embodiments, the emollient component is present in an amount of about 5% to about 15% by weight of the emulsion.

In some embodiments, the emollient component is present in an amount of about 7% to about 13% by weight of the emulsion.

In some embodiments, the emollient component comprises one or more substances independently selected from mineral oils and triglycerides.

In some embodiments, the emollient component comprises one or more substances independently selected from light mineral oil and medium chain triglycerides.

In some embodiments, the emollient component comprises one or more substances independently selected from light mineral oil, medium chain triglycerides, and dimethicone.

In some embodiments, the water is present in an amount of about 35% to about 65% by weight of the emulsion.

In some embodiments, the water is present in an amount of about 40% to about 60% by weight of the emulsion.

In some embodiments, the water is present in an amount of about 45% to about 55% by weight of the emulsion.

In some embodiments, the emulsifier component is present in an amount of about 1% to about 9% by weight of the emulsion.

In some embodiments, the emulsifier component is present in an amount of about 2% to about 6% by weight of the emulsion.

In some embodiments, the emulsifier component is present in an amount of about 3% to about 5% by weight of the emulsion.

In some embodiments, the emulsifier component is present in an amount of about 4% to about 7% by weight of the emulsion.

In some embodiments, the emulsion comprises an emulsifier component and a stiffening agent component, wherein the combined amount of emulsifier component and stiffening agent component is at least about 8% by weight of the emulsion.

In some embodiments, the emulsifier component comprises one or more substances independently selected from glyceryl fatty esters and sorbitan fatty esters.

In some embodiments, the emulsifier component comprises one or more substances independently selected from glyceryl stearate, and polysorbate 20.

In some embodiments, the emulsion further comprises a stabilizing agent component.

In some embodiments, the stabilizing agent component is present in an amount of about to about 5% by weight of the emulsion.

In some embodiments, the stabilizing agent component is present in an amount of about to about 2% by weight of the emulsion.

In some embodiments, the stabilizing agent component is present in an amount of about to about 0.5% by weight of the emulsion.

In some embodiments, the stabilizing agent component comprises one or more substances independently selected from polysaccharides.

In some embodiments, the stabilizing agent component comprises xanthan gum.

In some embodiments, the emulsion further comprises a solvent component.

In some embodiments, the solvent component is present in an amount of about 10% to about 35% by weight of the emulsion.

In some embodiments, the solvent component is present in an amount of about 15% to about 30% by weight of the emulsion.

In some embodiments, the solvent component is present in an amount of about 20% to about 25% by weight of the emulsion.

In some embodiments, the solvent component comprises one or more substances independently selected from alkylene glycols and polyalkylene glycols.

In some embodiments, the solvent component comprises one or more substances independently selected from propylene glycol and polyethylene glycol.

In some embodiments, the emulsion comprises:
from about 35% to about 65% of water by weight of the emulsion;
from about 10% to about 40% of an oil component by weight of the emulsion;
from about 1% to about 9% of an emulsifier component by weight of the emulsion;
from about 10% to about 35% of a solvent component by weight of the emulsion;
from about 0.05% to about 5% of a stabilizing agent component by weight of the emulsion; and
from about 0.5% to 1.5% of ruxolitinib on a free base basis by weight of the emulsion on a free base basis.

In some embodiments, the emulsion comprises:
from about 35% to about 65% of water by weight of the emulsion;
from about 10% to about 24% of an oil component by weight of the emulsion;
from about 1% to about 9% of an emulsifier component by weight of the emulsion;
from about 10% to about 35% of a solvent component by weight of the emulsion;
from about 0.05% to about 5% of a stabilizing agent component by weight of the emulsion; and
from about 0.5% to 1.5% of ruxolitinib on a free base basis by weight of the emulsion on a free base basis.
In some embodiments, the emulsion comprises:
from about 40% to about 60% of water by weight of the emulsion;
from about 15% to about 30% of an oil component by weight of the emulsion;
from about 2% to about 6% of an emulsifier component by weight of the emulsion;
from about 15% to about 30% of a solvent component by weight of the emulsion;
from about 0.1% to about 2% of a stabilizing agent component by weight of the emulsion; and
from about 0.5% to 1.5% of ruxolitinib on a free base basis by weight of the emulsion on a free base basis.

In some embodiments, the emulsion comprises:
from about 40% to about 60% of water by weight of the emulsion;
from about 15% to about 30% of an oil component by weight of the emulsion;
from about 2% to about 6% of an emulsifier component by weight of the emulsion;
from about 15% to about 24% of a solvent component by weight of the emulsion;
from about 0.1% to about 2% of a stabilizing agent component by weight of the emulsion; and
from about 0.5% to 1.5% of ruxolitinib on a free base basis by weight of the emulsion on a free base basis.

In some embodiments, the emulsion comprises:
from about 45% to about 55% of water by weight of the emulsion;
from about 15% to about 24% of an oil component by weight of the emulsion;
from about 3% to about 5% of an emulsifier component by weight of the emulsion;
from about 20% to about 25% of a solvent component by weight of the emulsion;
from about 0.3% to about 0.5% of a stabilizing agent component by weight of the emulsion; and
from about 0.5% to 1.5% of ruxolitinib on a free base basis by weight of the emulsion on a free base basis.

In some embodiments, the emulsion comprises:
from about 45% to about 55% of water by weight of the emulsion;
from about 15% to about 24% of an oil component by weight of the emulsion;
from about 4% to about 7% of an emulsifier component by weight of the emulsion;
from about 20% to about 25% of a solvent component by weight of the emulsion;
from about 0.3% to about 0.5% of a stabilizing agent component by weight of the emulsion; and
from about 0.5% to 1.5% of ruxolitinib on a free base basis by weight of the emulsion on a free base basis.

In some embodiments:
the oil component comprises one or more substances independently selected from petrolatums, fatty alcohols, mineral oils, triglycerides, and dimethicones;
the emulsifier component comprises one or more substances independently selected from glyceryl fatty esters and sorbitan fatty esters;
the solvent component comprises one or more substances independently selected from alkylene glycols and polyalkylene glycols; and
the stabilizing agent component comprises one or more substances independently selected from polysaccharides.

In some embodiments:
the oil component comprises one or more substances independently selected from white petrolatum, cetyl alcohol, stearyl alcohol, light mineral oil, medium chain triglycerides, and dimethicone;
the emulsifier component comprises one or more substances independently selected from glyceryl stearate and polysorbate 20;
the solvent component comprises one or more substances independently selected from propylene glycol and polyethylene glycol; and
the stabilizing agent component comprises xanthan gum.

In some embodiments, the emulsion comprises:
from about 35% to about 65% of water by weight of the emulsion;
from about 2% to about 15% of an occlusive agent component by weight of the emulsion;
from about 2% to about 8% of a stiffening agent component by weight of the emulsion;
from about 5% to about 15% of an emollient component by weight of the emulsion;
from about 1% to about 9% of an emulsifier component by weight of the emulsion;
from about 0.05% to about 5% of a stabilizing agent component by weight of the emulsion;
from about 10% to about 35% of a solvent component by weight of the emulsion; and
from about 0.5% to 1.5% of ruxolitinib on a free base basis by weight of the emulsion on a free base basis.

In some embodiments, the emulsion comprises:
from about 40% to about 60% of water by weight of the emulsion;
from about 5% to about 10% of an occlusive agent component by weight of the emulsion;
from about 2% to about 8% of a stiffening agent component by weight of the emulsion;
from about 7% to about 12% of an emollient component by weight of the emulsion;
from about 2% to about 6% of an emulsifier component by weight of the emulsion;
from about 0.1% to about 2% of a stabilizing agent by weight of the emulsion;
from about 15% to about 30% of a solvent component by weight of the emulsion; and
from about 0.5% to 1.5% of ruxolitinib on a free base basis by weight of the emulsion on a free base basis.

In some embodiments, the emulsion comprises:
from about 45% to about 55% of water by weight of the emulsion;
from about 5% to about 10% of an occlusive agent component by weight of the emulsion;
from about 3% to about 6% of a stiffening agent component by weight of the emulsion;
from about 7% to about 13% of an emollient component by weight of the emulsion;
from about 3% to about 5% of an emulsifier component by weight of the emulsion;
from about 0.3% to about 0.5% of a stabilizing agent component by weight of the emulsion;
from about 20% to about 25% of a solvent component by weight of the emulsion; and
from about 0.5% to 1.5% of ruxolitinib on a free base basis by weight of the emulsion on a free base basis.

In some embodiments, the emulsion comprises:
from about 45% to about 55% of water by weight of the emulsion;
from about 5% to about 10% of an occlusive agent component by weight of the emulsion;
from about 4% to about 7% of a stiffening agent component by weight of the emulsion;
from about 7% to about 13% of an emollient component by weight of the emulsion;
from about 4% to about 7% of an emulsifier component by weight of the emulsion;
from about 0.3% to about 0.5% of a stabilizing agent component by weight of the emulsion;
from about 20% to about 25% of a solvent component by weight of the emulsion; and
from about 0.5% to 1.5% of ruxolitinib on a free base basis by weight of the emulsion on a free base basis.

In some embodiments, the emulsion comprises:
from about 45% to about 55% of water by weight of the emulsion;
about 7% of an occlusive agent component by weight of the emulsion;
from about 4.5% to about 5% of a stiffening agent component by weight of the emulsion;
about 10% of an emollient component by weight of the emulsion;
from about 4% to about 4.5% of an emulsifier component by weight of the emulsion;
about 0.4% of a stabilizing agent component by weight of the emulsion;
about 22% of a solvent component by weight of the emulsion; and
from about 0.5% to 1.5% of ruxolitinib on a free base basis by weight of the emulsion on a free base basis.

In some embodiments, the combined amount of the stiffening agent component and the emulsifier component is at least about 8% by weight of the emulsion.

In some embodiments:
the occlusive agent component comprises a petrolatum;
the stiffening agent component comprises one or more substances independently selected from one or more fatty alcohols;
the emollient component comprises one or more substances independently selected from mineral oils and triglycerides;
the emulsifier component comprises one or more substances independently selected from glyceryl fatty esters and sorbitan fatty esters;
the stabilizing agent component comprises one or more substances independently selected from polysaccharides; and
the solvent component comprises one or more substances independently selected from alkylene glycols and polyalkylene glycols.

In some embodiments:
the occlusive agent component comprises white petrolatum;
the stiffening agent component comprises one or more substances independently selected from cetyl alcohol and stearyl alcohol;
the emollient component comprises one or more substances independently selected from light mineral oil, medium chain triglycerides, and dimethicone;
the emulsifier component comprises one or more substances independently selected from glyceryl stearate and polysorbate 20;
the stabilizing agent component comprises xanthan gum; and
the solvent component comprises one or more substances independently selected from propylene glycol and polyethylene glycol.

In some embodiments, the emulsion further comprises an antimicrobial preservative component.

In some embodiments, the antimicrobial preservative component is present in an amount of about 0.05% to about 3% by weight of the emulsion.

In some embodiments, the antimicrobial preservative component is present in an amount of about 0.1% to about 1% by weight of the emulsion.

In some embodiments, the antimicrobial preservative component comprises one or more substances independently selected from alkyl parabens and phenoxyethanol.

In some embodiments, the antimicrobial preservative component comprises one or more substances independently selected from methyl paraben, propyl paraben, and phenoxyethanol.

In some embodiments, the emulsion further comprises a chelating agent component.

In some embodiments, the chelating agent component comprises edetate disodium.

As used herein, the term “emulsifier component” refers, in one aspect, to a substance, or mixtures of substances that maintains an element or particle in suspension within a fluid medium. In some embodiments, the emulsifier component allows an oil phase to form an emulsion when combined with water. In some embodiments, the emulsifier component refers to one or more non-ionic surfactants.

As used herein, the term “occlusive agent component” refers to a hydrophobic agent or mixtures of hydrophobic agents that form an occlusive film on skin that reduces transepidermal water loss (TEWL) by preventing evaporation of water from the stratum corneum.

As used herein, the term “stiffening agent component” refers to a substance or mixture of substances that increases the viscosity and/or consistency of the cream or improves the rheology of the cream.

As used herein, the term “emollient component” refers to an agent that softens or soothes the skin or soothes an irritated internal surface.

As used herein, the term “stabilizing agent component” refers to a substance or mixture of substances that improves the stability of the cream and/or the compatibility of the components in the cram. In some embodiments, the stabilizing agent component prevents agglomeration of the emulsion and stabilizes the droplets in the oil-in-water emulsion.

As used herein, the term “solvent component” is a liquid substance or mixture of liquid substances capable of dissolving ruxolitinib di-hydrate in the cream. In some embodiments, the solvent component is a liquid substance or mixture of liquid substances in which ruxolitinib, or its pharmaceutically acceptable salt, has reasonable solubility. For example, solubilities of ruxolitinib free base are reported in Table 21 of US 2015/0250790, which is incorporated herein by reference in its entirety. Solubility information for ruxolitinib di-hydrate is shown in Tables 2 and 3 infra.

As used herein, the phrase “antimicrobial preservative component” is a substance or mixtures of substances which inhibits microbial growth in the cream.

As used herein, the phrase “chelating agent component” refers to a compound or mixtures of compounds that has the ability to bind strongly with metal ions.

As used herein, “% by weight of the emulsion” means the percent concentration of the component in the emulsion is on weight/weight basis. For example, 1% w/w of component A=[(mass of component A)/(total mass of the emulsion)]×100.

As used herein, the term “component” can mean one substance or a mixture of substances.

As used herein, the term “fatty acid” refers to an aliphatic acid that is saturated or unsaturated. In some embodiments, the fatty acid is in a mixture of different fatty acids. In some embodiments, the fatty acid has between about eight to about thirty carbons on average. In some embodiments, the fatty acid has about 12 to 20, 14-20, or 16-18 carbons on average. Suitable fatty acids include, but are not limited to, cetyl acid, stearic acid, lauric acid, myristic acid, erucic acid, palmitic acid, palmitoleic acid, capric acid, caprylic acid, oleic acid, linoleic acid, linolenic acid, hydroxystearic acid, 12-hydroxystearic acid, cetostearic acid, isostearic acid, sesquioleic acid, sesqui-9-octadecanoic acid, sesquiisooctadecanoic acid, behenic acid, isobehenic acid, and arachidonic acid, or mixtures thereof.

As used herein, the term “fatty alcohol” refers to an aliphatic alcohol that is saturated or unsaturated. In some embodiments, the fatty alcohol is in a mixture of different fatty alcohols. In some embodiments, the fatty alcohol has between about 12 to about 20, about 14 to about 20, or about 16 to about 18 carbons on average. Suitable fatty alcohols include, but are not limited to, stearyl alcohol, lauryl alcohol, palmityl alcohol, cetyl alcohol, capryl alcohol, caprylyl alcohol, oleyl alcohol, linolenyl alcohol, arachidonic alcohol, behenyl alcohol, isobehenyl alcohol, selachyl alcohol, chimyl alcohol, and linoleyl alcohol, or mixtures thereof.

As used herein, the term “polyalkylene glycol”, employed alone or in combination with other terms, refers to a polymer containing oxyalkylene monomer units, or copolymer of different oxyalkylene monomer units, wherein the alkylene group has 2 to 6, 2 to 4, or 2 to 3 carbon atoms. As used herein, the term “oxyalkylene”, employed alone or in combination with other terms, refers to a group of formula —O-alkylene-. In some embodiments, the polyalkylene glycol is polyethylene glycol.

As used herein, the term, “sorbitan fatty ester” includes products derived from sorbitan or sorbitol and fatty acids and, optionally, poly(ethylene glycol) units, including sorbitan esters and polyethoxylated sorbitan esters. In some embodiments, the sorbitan fatty ester is a polyethoxylated sorbitan ester.

As used herein, the term “sorbitan ester” refers to a compound, or mixture of compounds, derived from the esterification of sorbitol and at least one fatty acid. Fatty acids useful for deriving the sorbitan esters include, but are not limited to, those described herein. Suitable sorbitan esters include, but are not limited to, the Span™ series (available from Uniqema), which includes Span 20 (sorbitan monolaurate), 40 (sorbitan monopalmitate), 60 (sorbitan monostearate), 65 (sorbitan tristearate), 80 (sorbitan monooleate), and 85 (sorbitan trioleate). Other suitable sorbitan esters include those listed in R. C. Rowe and P. J. Shesky, Handbook of pharmaceutical excipients, (2006), 5th ed., which is incorporated herein by reference in its entirety.

As used herein, the term “polyethoxylated sorbitan ester” refers to a compound, or mixture thereof, derived from the ethoxylation of a sorbitan ester. The polyoxethylene portion of the compound can be between the fatty ester and the sorbitan moiety. As used herein, the term “sorbitan ester” refers to a compound, or mixture of compounds, derived from the esterification of sorbitol and at least one fatty acid. Fatty acids useful for deriving the polyethoyxlated sorbitan esters include, but are not limited to, those described herein. In some embodiments, the polyoxyethylene portion of the compound or mixture has about 2 to about 200 oxyethylene units. In some embodiments, the polyoxyethylene portion of the compound or mixture has about 2 to about 100 oxyethylene units. In some embodiments, the polyoxyethylene portion of the compound or mixture has about 4 to about 80 oxyethylene units. In some embodiments, the polyoxyethylene portion of the compound or mixture has about 4 to about 40 oxyethylene units. In some embodiments, the polyoxyethylene portion of the compound or mixture has about 4 to about 20 oxyethylene units. Suitable polyethoxylated sorbitan esters include, but are not limited to the Tween™ series (available from Uniqema), which includes Tween 20 (POE(20) sorbitan monolaurate), 21 (POE(4) sorbitan monolaurate), 40 (POE(20) sorbitan monopalmitate), 60

(POE(20) sorbitan monostearate), 60K (POE(20) sorbitan monostearate), 61 (POE(4) sorbitan monostearate), 65 (POE(20) sorbitan tristearate), 80 (POE(20) sorbitan monooleate), 80K (POE(20) sorbitan monooleate), 81 (POE(5) sorbitan monooleate), and 85 (POE(20) sorbitan trioleate). As used herein, the abbreviation “POE” refers to polyoxyethylene. The number following the POE abbreviation refers to the number of oxyethylene repeat units in the compound. Other suitable polyethoxylated sorbitan esters include the polyoxyethylene sorbitan fatty acid esters listed in R. C. Rowe and P. J. Shesky, Handbook of pharmaceutical excipients, (2006), 5th ed., which is incorporated herein by reference in its entirety. In some embodiments, the polyethoxylated sorbitan ester is a polysorbate. In some embodiments, the polyethoxylated sorbitan ester is polysorbate 20.

As used herein, the term “glyceryl fatty esters” refers to mono-, di- or triglycerides of fatty acids. The glyceryl fatty esters may be optionally substituted with sulfonic acid groups, or pharmaceutically acceptable salts thereof. Suitable fatty acids for deriving glycerides of fatty acids include, but are not limited to, those described herein. In some embodiments, the glyceryl fatty ester is a mono-glyceride of a fatty acid having 12 to 18 carbon atoms. In some embodiments, the glyceryl fatty ester is glyceryl stearate.

As used herein, the term “triglycerides” refers to a triglyceride of a fatty acid. In some embodiments, the triglyceride is medium chain triglycerides.

As used herein, the term “alkylene glycol” refers to a group of formula —O-alkylene-, wherein the alkylene group has 2 to 6, 2 to 4, or 2 to 3 carbon atoms. In some embodiments, the alkylene glycol is propylene glycol (1,2-propanediol).

As used herein, the term “polyethylene glycol” refers to a polymer containing ethylene glycol monomer units of formula -O-CH2-CH2-. Suitable polyethylene glycols may have a free hydroxyl group at each end of the polymer molecule, or may have one or more hydroxyl groups etherified with a lower alkyl, e.g., a methyl group. Also suitable are derivatives of polyethylene glycols having esterifiable carboxy groups. Polyethylene glycols useful in the present disclosure can be polymers of any chain length or molecular weight, and can include branching. In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 900. In some embodiments, the average molecular weight of the polyethylene glycol is about 400. Suitable polyethylene glycols include, but are not limited to polyethylene glycol-200, polyethylene glycol-300, polyethylene glycol-400, polyethylene glycol-600, and polyethylene glycol-900. The number following the dash in the name refers to the average molecular weight of the polymer.

### Methods

Ruxolitinib inhibits JAK1 and JAK2 and is selective for JAK1 over JAK3. An aspect of the present disclosure pertains to methods of treating a JAK-associated disease or disorder in an individual (e.g., patient) by administering to the individual in need of such treatment a therapeutically effective amount or dose of a solid form described herein, pharmaceutical formulation, or pharmaceutical composition of the present disclosure. JAK-associated diseases can include those characterized by expression of a mutant JAK2 such as those having at least one mutation in the pseudo-kinase domain (e.g., JAK2V617F).

Provided herein is a method of treating a disease in a patient in need thereof, comprising administering to the patient a pharmaceutical composition described herein, wherein the disease is myelofibrosis, polycythemia vera, acute graft versus host disease or chronic graft versus host disease.

Examples of JAK-associated diseases include myeloproliferative disorders (MPDS) such as polycythemia vera (PV), essential thrombocythemia (ET), myelofibrosis or myeloid metaplasia with myelofibrosis (MMM), chronic myelogenous leukemia (CML), chronic myelomonocytic leukemia (CMML), hypereosinophilic syndrome (FEES), systemic mast cell disease (SMCD), and the like. In some embodiments, the myeloproliferative disorder is myelofibrosis. In some embodiments, the myelofibrosis is primary myelofibrosis, post-polycythemia vera myelofibrosis, or post-essential thrombocythemia myelofibrosis. In some embodiments, the myeloproliferative disease is polycythemia vera. In some embodiments, the myeloproliferative disease is essential thrombocythemia.

In further embodiments, the JAK-associated disease is cancer including those characterized by solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, melanoma etc.), hematological cancers (e.g., lymphoma, leukemia such as acute lymphoblastic leukemia, or multiple myeloma), and skin cancer such as cutaneous T-cell lymphoma (CTCL) and cutaneous B-cell lymphoma. Example cutaneous T-cell lymphomas include Sezary syndrome and mycosis fungoides.

Examples of JAK-associated diseases include diseases involving the immune system including, for example, organ transplant rejection (e.g., allograft rejection and graft versus host disease). In some embodiments, the JAK-associated disease is graft versus host disease. In some embodiments, the JAK-associated disease is acute graft versus host disease. In some embodiments, the JAK-associated disease is chronic graft versus host disease.

Further examples of JAK-associated diseases include allergic conditions such as asthma, food allergies and rhinitis. Other examples of JAK-associated diseases include viral diseases such as Epstein Barr Virus (EBV), Hepatitis B, Hepatitis C, HIV, HTLV 1, Varicella-Zoster Virus (VZV), Human Papilloma Virus (HPV), and a coronavirus (e.g., SARS-COV-2).

Further JAK-associated diseases include inflammation and inflammatory diseases. Example inflammatory diseases include inflammatory diseases of the eye (e.g., iritis, uveitis, scleritis, conjunctivitis, or related disease), inflammatory diseases of the respiratory tract (e.g., the upper respiratory tract including the nose and sinuses such as rhinitis or sinusitis or the lower respiratory tract including bronchitis, chronic obstructive pulmonary disease, and the like), inflammatory myopathy such as myocarditis, and other inflammatory diseases. Other inflammatory diseases treatable by the compounds of the disclosure include systemic inflammatory response syndrome (SIRS) and septic shock.

Further examples of JAK-associated diseases include autoimmune diseases such as multiple sclerosis, rheumatoid arthritis, juvenile arthritis, type I diabetes, lupus, psoriasis, inflammatory bowel disease, ulcerative colitis, Crohn's disease, myasthenia gravis, immunoglobulin nephropathies, autoimmune thyroid disorders, and the like.

The JAK inhibitors described herein can further be used to treat ischemia reperfusion injuries or a disease or condition related to an inflammatory ischemic event such as stroke or cardiac arrest. The JAK inhibitors described herein can further be used to treat anorexia, cachexia, or fatigue such as that resulting from or associated with cancer. The JAK inhibitors described herein can further be used to treat restenosis, sclerodermitis, or fibrosis. The JAK inhibitors described herein can further be used to treat conditions associated with hypoxia or astrogliosis such as, for example, diabetic retinopathy, cancer, or neurodegeneration. See, e.g., Dudley, A. C. et al. Biochem. J. 2005, 390(Pt 2):427-36 and Sriram, K. et al. J. Biol. Chem. 2004, 279(19):19936-47. Epub 2004 Mar. 2.

The JAK inhibitors described herein can further be used to treat gout and increased prostate size due to, e.g., benign prostatic hypertrophy or benign prostatic hyperplasia.

Further examples of JAK-associated diseases include skin diseases. In some embodiments, provided herein is method of treating a skin disorder in a patient in need thereof, comprising administering to the patient a pharmaceutical composition described herein. In some embodiments, provided herein is method of treating a skin disorder in a patient in need thereof, comprising administering to an affected skin area of the patient a topical pharmaceutical formulation described herein. In some embodiments, provided herein is a method of treating a skin disorder, comprising applying a pharmaceutical composition described herein to an area of skin of a patient in need thereof.

In some embodiments, the skin disorder is an autoimmune skin disease. In some embodiments, the skin disorder is atopic dermatitis. In some embodiments, the skin disorder is vitiligo. In some embodiments, the autoimmune disease is an autoimmune bullous skin disorder such as pemphigus vulgaris (PV) or bullous pemphigoid (BP). In some embodiments, the skin disease is lichen planus. In some embodiments, the skin disease is prurigo nodularis. In some embodiments, the skin disease is hidradenitis suppurativa. In some embodiments, the skin disease is psoriasis. In some embodiments, the skin disease is psoriasis vulgaris or plaque psoriasis. In some embodiments, the skin disease is skin rash, skin irritation, or skin sensitization. In some embodiments, the skin disease is contact dermatitis or allergic contact dermatitis. In some embodiments, the skin disease is bullous pemphigoid.

The present disclosure further provides a method of treating dermatological side effects of other pharmaceuticals by administration of the compound of the disclosure. For example, numerous pharmaceutical agents result in unwanted allergic reactions which can manifest as acneiform rash or related dermatitis. Example pharmaceutical agents that have such undesirable side effects include anti-cancer drugs such as gefitinib, cetuximab, erlotinib, and the like. The formulations of the disclosure can be administered systemically or topically (e.g., localized to the vicinity of the dermatitis) in combination with (e.g., simultaneously or sequentially) the pharmaceutical agent having the undesirable dermatological side effect. In some embodiments, the formulation of the disclosure can be administered topically together with one or more other pharmaceuticals, where the other pharmaceuticals when topically applied in the absence of a formulation of the disclosure cause contact dermatitis, allergic contact sensitization, or similar skin disorder. Accordingly, formulation of the disclosure include topical formulations further comprising an additional pharmaceutical agent which can cause dermatitis, skin disorders, or related side effects.

In some embodiments, contacting a JAK with a solid form of the disclosure includes the administration of a compound of the present disclosure to an individual or patient, such as a human, having a JAK, as well as, for example, introducing a compound of the disclosure into a sample containing a cellular or purified preparation containing the JAK.

As used herein, the term “individual” or “patient,” used interchangeably, refers to any animal, including mammals, preferably mice, rats, other rodents, rabbits, dogs, cats, swine, cattle, sheep, horses, or primates, and most preferably humans. In some embodiments, the patient is a human patient.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder; and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compound and composition described herein can prevent a disease, condition, or disorder. Preventing or prevention of a disease, condition or disorder refers to administering the compound or composition described herein in an individual who may be predisposed to the disease, condition or disorder but does not yet experience or display the pathology or symptomatology of the disease.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:
(1) inhibiting the disease; for example, inhibiting a disease, condition or disorder in an individual who is experiencing or displaying the pathology or symptomatology of the disease, condition or disorder (i.e., arresting further development of the pathology and/or symptomatology); and
(2) ameliorating the disease; for example, ameliorating a disease, condition or disorder in an individual who is experiencing or displaying the pathology or symptomatology of the disease, condition or disorder (i.e., reversing the pathology and/or symptomatology).

### Combination Therapies

One or more additional pharmaceutical agents such as, for example, chemotherapeutics, anti-inflammatory agents, steroids, immunosuppressants, as well as Bcr-Abl, Flt-3, RAF and FAK kinase inhibitors such as, for example, those described in WO 2006/056399, or other agents can be used in combination with the composition described herein for treatment of JAK-associated diseases, disorders or conditions. The one or more additional pharmaceutical agents can be administered to a patient simultaneously or sequentially.

Example chemotherapeutic include proteosome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include the compounds, and pharmaceutically acceptable salts thereof, of the genera and species disclosed in U.S. Pat. No. 5,521,184, WO 04/005281, and US Pat. No. 7,745,437.

Example suitable Flt-3 inhibitors include compounds, and their pharmaceutically acceptable salts, as disclosed in WO 03/037347, WO 03/099771, and WO 04/046120.

Example suitable RAF inhibitors include compounds, and their pharmaceutically acceptable salts, as disclosed in WO 00/09495 and WO 05/028444.

Example suitable FAK inhibitors include compounds, and their pharmaceutically acceptable salts, as disclosed in WO 04/080980, WO 04/056786, WO 03/024967, WO 01/064655, WO 00/053595, and WO 01/014402.

In some embodiments, the compositions described herein can be used in combination with one or more other kinase inhibitors including imatinib, particularly for treating patients resistant to imatinib or other kinase inhibitors.

In some embodiments, a corticosteroid such as dexamethasone is administered to a patient in combination with the composition described herein where the dexamethasone is administered intermittently as opposed to continuously.

### Kits

The present disclosure also includes pharmaceutical kits useful, for example, in the treatment or prevention of JAK-associated diseases or disorders, such as cancer, which include one or more containers containing a compound or composition. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### Additional Embodiments

Embodiment 1: A solid form, which is crystalline ruxolitinib di-hydrate:

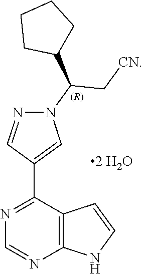

Embodiment 2: The solid form of embodiment 1, wherein the solid form is substantially isolated.
Embodiment 3: The solid form of embodiments 1 or 2, wherein the solid form is characterized by having at least one XRPD peak, in terms of 2-theta (±0.2 degrees), selected from 6.9, 10.6, 11.6, 12.9, 15.1, 15.4, 19.0, 21.8, 22.7, 23.1, 24.8, and 25.7 degrees.
Embodiment 4: The solid form of embodiments 1 or 2, wherein the solid form is characterized by having at least two XRPD peaks, in terms of 2-theta (±0.2 degrees), selected from 6.9, 10.6, 11.6, 12.9, 15.1, 15.4, 19.0, 21.8, 22.7, 23.1, 24.8, and 25.7 degrees.
Embodiment 5: The solid form of embodiments 1 or 2, wherein the solid form is characterized by having at least three XRPD peaks, in terms of 2-theta (±0.2 degrees), selected from 6.9, 10.6, 11.6, 12.9, 15.1, 15.4, 19.0, 21.8, 22.7, 23.1, 24.8, and 25.7 degrees.
Embodiment 6: The solid form of embodiments 1 or 2, wherein the solid form is characterized by having at least four XRPD peaks, in terms of 2-theta (±0.2 degrees), selected from 6.9, 10.6, 11.6, 12.9, 15.1, 15.4, 19.0, 21.8, 22.7, 23.1, 24.8, and 25.7 degrees.
Embodiment 7: The solid form of embodiments 1 or 2, wherein the solid form is characterized by having at least five XRPD peaks, in terms of 2-theta (±0.2 degrees), selected from 6.9, 10.6, 11.6, 12.9, 15.1, 15.4, 19.0, 21.8, 22.7, 23.1, 24.8, and 25.7 degrees.
Embodiment 8: The solid form of embodiments 1 or 2, wherein the solid form is characterized by having at least six XRPD peaks, in terms of 2-theta (±0.2 degrees), selected from 6.9, 10.6, 11.6, 12.9, 15.1, 15.4, 19.0, 21.8, 22.7, 23.1, 24.8, and 25.7 degrees.
Embodiment 9: The solid form of embodiments 1 or 2, wherein the solid form is characterized by having XRPD peaks, in terms of 2-theta (±0.2 degrees), selected from 6.9, 11.6, 12.9, 15.1, 15.4, 19.0, 21.8, 22.7, 23.1, 24.8, and 25.7 degrees.
Embodiment 10: The solid form of embodiments 1 or 2, wherein the solid form is characterized by having XRPD peaks, in terms of 2-theta (±0.2 degrees), selected from 19.0, 22.7, and 23.1 degrees.
Embodiment 11: The solid form of embodiment 10, wherein the solid form is characterized by having further XRPD peaks, in terms of 2-theta (±0.2 degrees), selected from 10.6 and 15.4 degrees.
Embodiment 12: The solid form of embodiment 11, wherein the solid form is characterized by having further XRPD peaks, in terms of 2-theta (±0.2 degrees), selected from 11.6 and 25.7 degrees.
Embodiment 13: The solid form of embodiment 12, wherein the solid form is characterized by having further XRPD peaks, in terms of 2-theta (±0.2 degrees), selected from 6.9 and 21.8 degrees.
Embodiment 14: The solid form of embodiment 13, wherein the solid form is characterized by having further XRPD peaks, in terms of 2-theta (±0.2 degrees), selected from 12.9, 15.1, and 24.8 degrees.
Embodiment 15: The solid form of any one of embodiments 1-14, wherein the solid form is characterized by having an XRPD pattern with characteristic peaks as substantially shown in FIG. 2.
Embodiment 16: The solid form of any one of embodiments 1-14, wherein the solid form is characterized by having an XRPD pattern with characteristic peaks as substantially shown in FIG. 9.
Embodiment 17: The solid form of any one of embodiments 1-16, wherein the solid form is characterized by single crystal x-ray diffraction having a P212121 space group and cell formula units (Z) of 8.
Embodiment 18: The solid form of embodiment 17, wherein the solid form has unit cell parameters: a is about 9.97 Å, b is about 15.18 Å, c is about 23.64 Å, α is about 90°, β is about 90°, and γ is about 90°.
Embodiment 19: The solid form of any one of embodiments 1-18, wherein the solid form is characterized by having an endothermic peak with an onset temperature (±5° C.) at 61° C. and a maximum temperature (±5° C.) at 67° C., in a DSC thermogram.
Embodiment 20: The solid form of any one of embodiments 1-18, wherein the solid form is characterized by having a first endothermic peak with onset temperature (±5° C.) at 68° C. and a maximum temperature (±5° C.) at 72° C., and a second endothermic peak with a maximum temperature (±5° C.) at 110° C., and in a DSC thermogram.
Embodiment 21: The solid form of any one of embodiments 1-18, wherein the solid form is characterized by having a DSC thermogram substantially as depicted in FIG. 3.
Embodiment 22: The solid form of any one of embodiments 1-18, wherein the solid form is characterized by having a DSC thermogram substantially as depicted in FIG. 10.
Embodiment 23: The solid form of any one of embodiments 1-22, wherein the solid form is characterized by having a TGA thermogram substantially as depicted in FIG. 4.
Embodiment 24: The solid form of any one of embodiments 1-22, wherein the solid form is characterized by having a TGA thermogram substantially as depicted in FIG. 11.
Embodiment 25: A solid form, which is anhydrous crystalline ruxolitinib free base.
Embodiment 26: The solid form of embodiment 25, wherein the solid form is substantially isolated.
Embodiment 27: The solid form of embodiments 25 or 26, wherein the solid form is characterized by having at least one XRPD peak, in terms of 2-theta (±0.2 degrees), selected from 7.2, 11.5, 11.6, 13.2, 14.0, 15.4, 15.7, 18.2, 19.1, 19.6, 22.0, and 23.9 degrees.
Embodiment 28: The solid form of embodiments 25 or 26, wherein the solid form is characterized by having XRPD peaks, in terms of 2-theta (±0.2 degrees), selected from 7.2, 11.5, 11.6, 13.2, 14.0, 15.4, 15.7, 18.2, 19.1, 19.6, 22.0, and 23.9 degrees.
Embodiment 29: The solid form of embodiments 25 or 26, wherein the solid form is characterized by having XRPD peaks, in terms of 2-theta (±0.2 degrees), selected from 7.2, 13.2, and 15.8, degrees.
Embodiment 30: The solid form of embodiment 29, wherein the solid form is characterized by having further XRPD peaks, in terms of 2-theta (±0.2 degrees), selected from 19.6 and 23.9 degrees.
Embodiment 31: The solid form of embodiment 30, wherein the solid form is characterized by having further XRPD peaks, in terms of 2-theta (±0.2 degrees), selected from 11.5 and 15.4 degrees.
Embodiment 32: The solid form of embodiment 31, wherein the solid form is characterized by having further XRPD peaks, in terms of 2-theta (±0.2 degrees), selected from 11.6 and 19.1 degrees.
Embodiment 33: The solid form of any one of embodiments 25-32, wherein the solid form is characterized by having an XRPD pattern with characteristic peaks as substantially shown in FIG. 13.
Embodiment 34: The solid form of any one of embodiments 25-33, wherein the solid form is characterized by having an endothermic peak with an onset temperature (±5° C.) at 83° C. and a maximum temperature (±5° C.) at 93° C., in a DSC thermogram.
Embodiment 35: The solid form of any one of embodiments 25-33, wherein the solid form is characterized by having an endothermic peak with an onset temperature (±5° C.) at 81° C. and a maximum temperature (±5° C.) at 91° C., in a DSC thermogram.
Embodiment 36: The solid form of any one of embodiments 25-33, wherein the solid form is characterized by having a DSC thermogram substantially as depicted in FIG. 15 or FIG. 16.
Embodiment 37: The solid form of any one of embodiments 25-36, wherein the solid form is characterized by having a TGA thermogram substantially as depicted in FIG. 17 or FIG. 18.
Embodiment 38: A process of preparing a solid form, which is ruxolitinib di-hydrate:

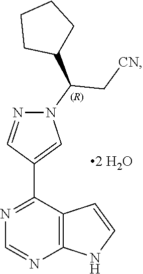

comprising contacting ruxolitinib free base with water.
Embodiment 39: A process of preparing a solid form, which is ruxolitinib di-hydrate:

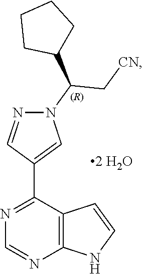

comprising isolating the solid form from a solution comprising ruxolitinib free base and an aqueous solvent component.
Embodiment 40: The process of embodiment 39, wherein said isolating comprises crystallizing the solid form from a solution comprising ruxolitinib free base and an aqueous solvent component.
Embodiment 41: The process of embodiment 40, wherein said crystallizing comprises cooling the solution to crystallize the solid form.
Embodiment 42: The process of embodiment 40, wherein said crystallizing comprises:
a) heating the solution comprising ruxolitinib free base and an aqueous solvent component; and
b) after said heating, cooling the solution to crystallize the solid form.
   Embodiment 43: The process of embodiment 40, wherein said crystallizing comprises:
a) heating the solution comprising ruxolitinib free base and an aqueous solvent component;
b) after said heating, cooling the solution;
c) after said cooling, adding seeds of crystalline ruxolitinib di-hydrate to the solution; and
d) after said adding of said seeds, stirring the solution to crystallize the solid form.
   Embodiment 44: The process of any one of embodiments 39-43, wherein the solution is formed using amorphous ruxolitinib free base.
   Embodiment 45: The process of any one of embodiments 42-44, wherein in step a), the solution comprising ruxolitinib free base and a solvent mixture is heated to a temperature of from about 40° C. to about 80° C.
   Embodiment 46: The process of any one of embodiments 42-44, wherein in step a), the solution comprising ruxolitinib free base and a solvent mixture is heated to a temperature of from about 50° C. to about 70° C.
   Embodiment 47: The process of any one of embodiments 42-44, wherein in step a), the solution comprising ruxolitinib free base and a solvent mixture is heated to a temperature of from about 55° C. to about 65° C.
   Embodiment 48: The process of any one of embodiments 42-47, wherein in step b), the solution is cooled to a temperature of from about 10° C. to about 40° C.
   Embodiment 49: The process of any one of embodiments 42-47, wherein in step b), the solution is cooled to a temperature of from about 15° C. to about 35° C.
   Embodiment 50: The process of any one of embodiments 42-47, wherein in step b), the solution is cooled to a temperature of from about 20° C. to about 30° C.
   Embodiment 51: The process of any one of embodiments 42-47, wherein in step b), the solution is cooled to a temperature of about ambient temperature.
   Embodiment 52: The process of any one of embodiments 43-51, wherein in step d), the solution is stirred for about 1 to about 30 hours.
   Embodiment 53: The process of any one of embodiments 43-51, wherein in step d), the solution is stirred for about 10 to about 20 hours.
   Embodiment 54: The process of any one of embodiments 43-51, wherein in step d), the solution is stirred for about 14 to about 18 hours.
   Embodiment 55: The process of any one of embodiments 39-54, wherein the aqueous solvent component is water.
   Embodiment 56: The process of any one of embodiments 39-54, wherein the aqueous solvent component comprises a polar protic solvent and water.
   Embodiment 57: The process of embodiment 56, wherein the polar protic solvent is an alcohol.
   Embodiment 58: The process of embodiment 56, wherein the polar protic solvent is a C₁₋₆ alcohol.
   Embodiment 59: The process of embodiment 58, wherein the C₁₋₆ alcohol is isopropanol.
   Embodiment 60: The process of any one of embodiments 56-59, wherein the volume to volume ratio of the polar protic solvent to the water is about 1 to 0.1 to about 1 to 10.
   Embodiment 61: The process of any one of embodiments 56-59, wherein the volume to volume ratio of polar protic solvent to water is about 1 to 0.5 to about 1 to 5.
   Embodiment 62: The process of any one of embodiments 56-59, wherein the volume to volume ratio of polar protic solvent to water is about 1 to 1 to about 1 to 3.
   Embodiment 63: The process of any one of embodiments 56-59, wherein the volume to volume ratio of polar protic solvent to water is about 1 to 2 to about 1 to 2.5.
   Embodiment 64: The process of any one of embodiments 38-63, wherein the ruxolitinib free base is prepared by a process comprising reacting ruxolitinib phosphate:

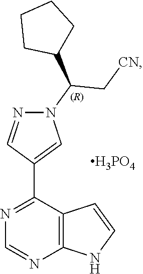

with a base in a solvent component.
Embodiment 65: The process of embodiment 64, wherein the ruxolitinib free base is amorphous.
Embodiment 66: The process of embodiments 64 or 65, wherein the reacting of ruxolitinib phosphate with a base comprises using from about 1 to about 10 molar equivalents of the base relative to ruxolitinib phosphate.
Embodiment 67: The process of embodiments 64 or 65, wherein the reacting of ruxolitinib phosphate with a base comprises using from about 1 to about 5 molar equivalents of the base relative to ruxolitinib phosphate.
Embodiment 68: The process of embodiments 64 or 65, wherein the reacting of ruxolitinib phosphate with a base comprises using from about 1 to about 3 molar equivalents of the base relative to ruxolitinib phosphate.
Embodiment 69: The process of embodiments 64 or 65, wherein the reacting of ruxolitinib phosphate with a base comprises using from about 2 to about 10 molar equivalents of the base relative to ruxolitinib phosphate.
Embodiment 70: The process of embodiments 64 or 65, wherein the reacting of ruxolitinib phosphate with a base comprises using from about 2 to about 5 molar equivalents of the base relative to ruxolitinib phosphate.
Embodiment 71: The process of embodiments 64 or 65, wherein the reacting of ruxolitinib phosphate with a base comprises using from about 2 to about 3 molar equivalents of the base relative to ruxolitinib phosphate.
Embodiment 72: The process of any one of embodiments 64-71, wherein the reacting of ruxolitinib phosphate with a base comprises using an amount of the base sufficient to generate a pH of about 7.5 to about 8.
Embodiment 73: The process of any one of embodiments 64-71, wherein the reacting of ruxolitinib phosphate with a base comprises using an amount of the base sufficient to generate a pH of about 7 to about 8.
Embodiment 74: The process of any one of embodiments 64-73, wherein the base is a hydroxide base.
Embodiment 75: The process of any one of embodiments 64-73, wherein the base is an alkali metal hydroxide or an alkaline earth metal hydroxide.
Embodiment 76: The process of any one of embodiments 64-73, wherein the base is KOH.
Embodiment 77: The process of any one of embodiments 64-73, wherein the base is NaOH.
Embodiment 78: The process of any one of embodiments 64-77, wherein the solvent component comprises water.
Embodiment 79: The process of any one of embodiments 64-77, wherein the solvent component comprises one or more aprotic solvents and water.
Embodiment 80: The process of any one of embodiments 64-77, wherein the solvent component comprises water, an ester solvent, a halogenated solvent, or a mixture thereof.
Embodiment 81: The process of embodiment 80, wherein the ester solvent is ethyl acetate.
Embodiment 82: The process of embodiment 80, wherein the halogenated solvent is dichloromethane.
Embodiment 83: The process of embodiment 80, wherein the solvent component comprises ethyl acetate, dichloromethane, and water.
Embodiment 84: The process of any one of embodiments 64-83, wherein the ruxolitinib phosphate in the solvent component is cooled to a temperature of from about 0° C. to about 10° C.
Embodiment 85: The process of any one of embodiments 64-83, wherein the ruxolitinib phosphate in the solvent component is cooled to a temperature of from about 0° C. to about 5° C.
Embodiment 86: A process of preparing a solid form, which is anhydrous crystalline ruxolitinib free base comprising drying crystalline ruxolitinib di-hydrate.
Embodiment 87: The process of embodiment 86, wherein the drying comprising drying crystalline ruxolitinib di-hydrate in ajar with desiccant at about room temperature.
Embodiment 88: The process of embodiment 87, wherein the desiccant is P₂O₅.
Embodiment 89: A solid form, which is ruxolitinib di-hydrate, which is prepared by the process of any one of embodiments 38-85.
Embodiment 90: A solid form, which is anhydrous crystalline ruxolitinib, which is prepared by the process of any one of embodiments 86-88.
Embodiment 91: A pharmaceutical composition comprising the solid form of any one of embodiments 1-37, 89 and 90.
Embodiment 92: The pharmaceutical composition of embodiment 91, which is an oral dosage form.
Embodiment 93: The pharmaceutical composition of embodiment 92, wherein the oral dosage form is an immediate dosage form.
Embodiment 94: The pharmaceutical composition of embodiment 93, wherein the ruxolitinib di-hydrate is present in an amount of about 5 to about 25 mg on a free base basis.
Embodiment 95: The pharmaceutical composition of embodiment 93, wherein the ruxolitinib di-hydrate is present in an amount of about 5 mg, about 10 mg, about 15 mg, about 20 mg, or about 25 mg of ruxolitinib di-hydrate on a free base basis.
Embodiment 96: The pharmaceutical composition of embodiment 93, wherein the oral dosage form is a sustained-release dosage form.
Embodiment 97: The pharmaceutical composition of embodiment 96, wherein the ruxolitinib di-hydrate is present in an amount of about 10 to about 50 mg on a free base basis.
Embodiment 98: The pharmaceutical composition of embodiment 96, wherein the ruxolitinib di-hydrate is present in an amount of about 10 mg, about 20 mg, about 30 mg, about mg, or about 50 mg of ruxolitinib di-hydrate on a free base basis.
Embodiment 99: The pharmaceutical composition of embodiment 91, wherein the composition is a topical formulation.
Embodiment 100: The pharmaceutical composition of embodiment 99, wherein the topical formulation is a cream formulation.
Embodiment 101: The pharmaceutical composition of embodiment 100, wherein the cream formulation comprises an oil-in-water emulsion.
Embodiment 102: The pharmaceutical composition of embodiment 101, wherein the cream formulation is prepared by incorporating ruxolitinib di-hydrate in the oil-in-water emulsion.
Embodiment 103: A topical pharmaceutical formulation, comprising ruxolitinib free base and a solvent component, wherein the formulation is prepared by dissolving the solid form of any one of embodiments 1-37, 89 and 90 in a solvent component.
Embodiment 104: The topical pharmaceutical formulation of any one of embodiments 99-103, wherein the ruxolitinib free base is present in an amount of about 0.5% to about 1.5% of ruxolitinib free base by weight of the formulation.
Embodiment 105: The topical pharmaceutical formulation of embodiment 104, wherein the ruxolitinib free base is present in an amount of about 1.5% of ruxolitinib free base by weight of the formulation.
Embodiment 106: The topical pharmaceutical formulation of any one of embodiments 103-105, wherein the topical pharmaceutical formulation comprises ruxolitinib di-hydrate, which is present in an amount of less than about 0.9%, less than about 0.8%, less than about 0.7%, less than about 0.6%, less than about 0.5%, less than about 0.4%, less than about 0.3%, less than about 0.2%, less than about 0.1%, less than about 0.09%, less than about 0.08%, less than about less than about 0.06%, less than about 0.05%, less than about 0.04%, less than about 0.03%, less than about 0.02%, less than about 0.01%, less than about 0.009%, less than about less than about 0.007%, less than about 0.006%, less than about 0.005%, less than about less than about 0.003%, less than about 0.002%, or less than about 0.001% on a free base basis by weight of the formulation.
Embodiment 107: A topical pharmaceutical formulation, comprising (a) ruxolitinib free base in an amount of about 0.5% to about 1.5% by weight of the formulation; and (b) ruxolitinib di-hydrate, which is present in an amount of less than about 0.9%, less than about 0.8%, less than about 0.7%, less than about 0.6%, less than about 0.5%, less than about 0.4%, less than about less than about 0.2%, less than about 0.1%, less than about 0.09%, less than about 0.08%, less than about 0.07%, less than about 0.06%, less than about 0.05%, less than about 0.04%, less than about 0.008%, less than about 0.007%, less than about 0.006%, less than about 0.005%, less than about 0.004%, less than about 0.003%, less than about 0.002%, or less than about 0.001% on a free base basis by weight of the formulation.
Embodiment 108: The topical pharmaceutical formulation of any one of embodiments 99-107, wherein the topical pharmaceutical formulation is prepared at a large batch size.
Embodiment 109: The topical pharmaceutical formulation of any one of embodiments 103-107, wherein the topical pharmaceutical formulation is prepared at a batch size of 1000 kg or higher.
Embodiment 110: A topical pharmaceutical formulation, comprising (a) ruxolitinib, or a pharmaceutically acceptable salt thereof, and (b) ruxolitinib di-hydrate, which is present in an amount of less than about 0.9%, less than about 0.8%, less than about 0.7%, less than about less than about 0.5%, less than about 0.4%, less than about 0.3%, less than about 0.2%, less than about 0.1%, less than about 0.09%, less than about 0.08%, less than about 0.07%, less than about 0.06%, less than about 0.05%, less than about 0.04%, less than about 0.03%, less than about 0.02%, less than about 0.01%, less than about 0.009%, less than about 0.008%, less than about 0.007%, less than about 0.006%, less than about 0.005%, less than about 0.004%, less than about 0.003%, less than about 0.002%, or less than about 0.001% on a free base basis by weight of the formulation.
Embodiment 111: The topical pharmaceutical formulation of embodiment 110, wherein the topical pharmaceutical formulation is prepared at a large batch size.
Embodiment 112: The topical pharmaceutical formulation of embodiment 110, wherein the topical pharmaceutical formulation is prepared at a batch size of 1000 kg or higher.
Embodiment 113: The topical pharmaceutical formulation of any one of embodiments 110-112, wherein the ruxolitinib, or the pharmaceutically acceptable salt thereof, is present in an amount of about 0.5% to about 1.5% on a free base basis by weight of the formulation.
Embodiment 114: The topical pharmaceutical formulation of embodiment 9113 wherein the ruxolitinib, or the pharmaceutically acceptable salt thereof, is present in an amount of about 1.5% on a free base basis by weight of formulation.
Embodiment 115: The topical pharmaceutical formulation of any one of embodiments 110-114, wherein the ruxolitinib, or the pharmaceutically acceptable salt thereof, is ruxolitinib phosphate.
Embodiment 116: A topical pharmaceutical formulation, comprising ruxolitinib, or a pharmaceutically acceptable salt thereof, wherein the formulation comprises less than about less than about 0.8%, less than about 0.7%, less than about 0.6%, less than about 0.5%, less than about 0.4%, less than about 0.3%, less than about 0.2%, less than about 0.1%, less than about 0.09%, less than about 0.08%, less than about 0.07%, less than about 0.06%, less than about 0.05%, less than about 0.04%, less than about 0.03%, less than about 0.02%, less than about 0.01%, less than about 0.009%, less than about 0.008%, less than about 0.007%, less than about 0.006%, less than about 0.005%, less than about 0.004%, less than about 0.003%, less than about 0.002%, or less than about 0.001% of ruxolitinib di-hydrate on a free base basis by weight of the formulation, wherein the formulation is prepared at a large batch size.
Embodiment 117: The topical pharmaceutical formulation of embodiment 116, wherein the ruxolitinib, or the pharmaceutically acceptable salt thereof, is present in an amount of about to about 1.5% of on a free base basis by weight of the formulation.
Embodiment 118: The topical pharmaceutical formulation of embodiment 117, wherein the ruxolitinib, or the pharmaceutically acceptable salt thereof, is present in an amount of about 1.5% on a free base basis by weight of formulation.
Embodiment 119: The topical pharmaceutical formulation of any one of embodiments 116-118, wherein the ruxolitinib, or the pharmaceutically acceptable salt thereof, is ruxolitinib phosphate.
Embodiment 120: The topical pharmaceutical formulation of any one of embodiments 116-119, wherein the batch size is 1000 kg or higher.
Embodiment 121: A process for releasing a batch of the topical pharmaceutical formulation of any one of embodiments 116-120, comprising (i) testing a sample of the topical pharmaceutical formulation for the absence of crystalline ruxolitinib di-hydrate; and, if the sample passes the test in step (i), then: (ii) releasing the batch for public use.
Embodiment 122: The process of embodiment 121, wherein the testing comprises observing a sample of the formulation under a light microscope in order to detect the absence or presence of crystals, wherein the sample passes the test when crystals are not detected.
Embodiment 123: A method of treating a disease in a patient in need thereof, comprising administering to the patient a pharmaceutical composition of any one of embodiments 91-115, wherein the disease is myelofibrosis, polycythemia vera, acute graft versus host disease or chronic graft versus host disease.
Embodiment 124: A method of treating a skin disorder in a patient in need thereof, comprising administering to the patient a pharmaceutical composition of any one of embodiments 91-115.
Embodiment 125: A method of treating a skin disorder in a patient in need thereof, comprising administering to an affected skin area of the patient a topical pharmaceutical formulation of any one of embodiments 99-115.
Embodiment 126: The method of any one of embodiments 124-125, wherein the skin disorder is an autoimmune skin disease.
Embodiment 127: The method of any one of embodiments 124-125, wherein the skin disorder is atopic dermatitis.
Embodiment 128: The method of any one of embodiments 124-125, wherein the skin disorder is lichen planus.
Embodiment 129: The method of any one of embodiments 124-125, wherein the skin disorder is hidradenitis suppurativa.
Embodiment 130: The method of any one of embodiments 124-125, wherein the skin disorder is psoriasis.
Embodiment 131: The method of any one of embodiments 124-125, wherein the skin disorder is plaque psoriasis.
Embodiment 132: The method of any one of embodiments 124-125, wherein the skin disorder is skin rash, skin irritation, or skin sensitization.
Embodiment 133: The method of any one of embodiments 124-125, wherein the skin disorder is contact dermatitis or allergic contact dermatitis.
Embodiment 134: The method of any one of embodiments 124-125, wherein the skin disorder is bullous pemphigoid.
Embodiment 135: The method of any one of embodiments 124-134, the patient is a human patient.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

The disclosure will be described in greater detail by way of specific examples. The following examples are offered for illustrative purposes, and are not intended to limit the disclosure in any manner. Those of skill in the art will readily recognize a variety of noncritical parameters which can be changed or modified to yield essentially the same results.

### EXAMPLES

The crystalline di-hydrate form of ruxolitinib was discovered during scale-up work to 3200 kg of a topical product having 1.5% (w/w) on a free base basis of ruxolitinib phosphate (prepared by methods analogous to those at smaller scale described in Example 16) and were isolated by diluting the product with water, filtering, washing with water, and drying. Two approaches have been utilized for preparation of crystalline ruxolitinib di-hydrate. The first approach is to generate crystalline ruxolitinib di-hydrate from the isolated amorphous ruxolitinib free base, Scheme 1, Example 1. In this approach, crystalline ruxolitinib phosphate salt is neutralized by a base, such as an aqueous potassium hydroxide (KOH) solution. The resulting ruxolitinib free base is then extracted into an organic solvent (EtOAc or DCM) or an organic solvent system (EtOAc and DCM). After distillation to remove the solvent(s), ruxolitinib free base is obtained as an amorphous solid. The amorphous ruxolitinib free base is then crystallized in a mixed solvent system, such as isopropanol (IPA) and water, to generate crystalline ruxolitinib di-hydrate.

The second approach to prepare crystalline ruxolitinib di-hydrate is to use the in-situ generated ruxolitinib free base for crystallization in aqueous isopropanol, Scheme 2, Example 2. In this approach, ruxolitinib free base does not need to be isolated. Instead, a solution of ruxolitinib free base in DCM or ethyl acetate (EtOAc) or a mixture of EtOAc and DCM is solvent switched to isopropanol (IPA). After addition of water and seeding with ruxolitinib di-hydrate crystals isolated using the previous approach, crystalline ruxolitinib di-hydrate is generated.

Preparation and isolation of ruxolitinib phosphate can be found in WO2008/157208, which is incorporated herein by reference in its entirety. Preparation and isolation of Compound 3 L-tartrate di-hydrate, Compound 2 chloride hydrochloride, and ruxolitinib free base can also be found in US-Patent Publ. 20220056035, which is incorporated herein by reference in its entirety.

### Example 1. Preparation of Crystalline Ruxolitinib Di-Hydrate From the Isolated Amorphous Ruxolitinib Free Base

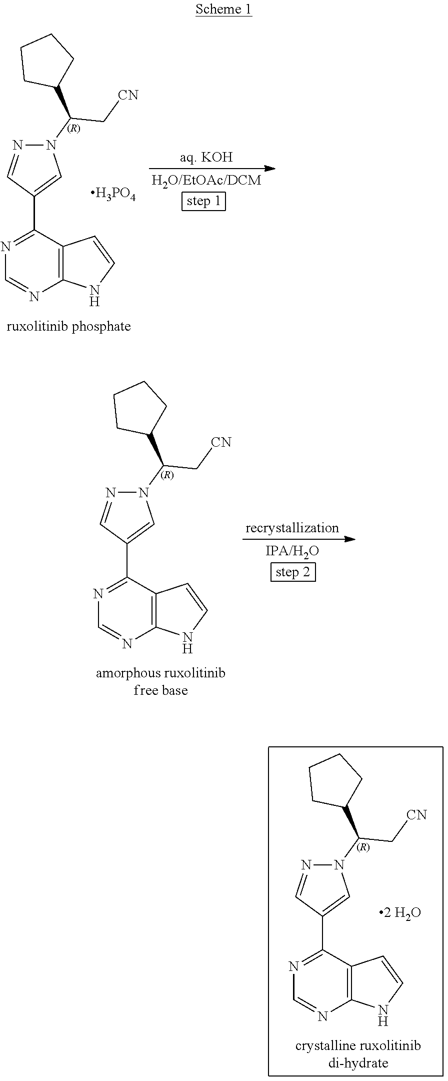

### Step 1: Neutralization of Ruxolitinib Phosphate and Isolation of the Amorphous Ruxolitinib Free Base.

A suspension of crystalline ruxolitinib phosphate, 116.14 g, 287.2 mmol) in ethyl acetate (EtOAc, 400 mL), water (350 mL), and dichloromethane (DCM, 1500 mL) was cooled to 0-5° C. in an ice bath before an aqueous solution of 3M potassium hydroxide (KOH, 200 mL, 600 mmol) was gradually charged. After neutralization (pH 7.5-8.0), the original suspension became a solution with two phases. The two phases were separated and the organic phase was washed with water (500 mL) and dried with magnesium sulfate (MgSO₄). After filtration to remove the drying agent, the organic solution was concentrated under reduced pressure to dryness. The resulting amber syrup was further dried under vacuum to afford ruxolitinib free base as an off-white to light-yellow amorphous powder, which was used in the subsequent crystallization process without further purification. For the amorphous ruxolitinib free base: ¹H NMR (DMSO-d₆, 400 MHz) δ 12.10 (br. s, 1H), 8.78 (s, 1H), 8.67 (s, 1H), 8.36 (s, 1H), 7.58 (dd, 1H, J=2.3, 3.4 Hz), 6.97 (dd, 1H, J=1.5, 3.6 Hz), 4.50 (td, 1H, J=9.7, 4.2 Hz), 3.26 (dd, 1H, J=17.5, 10.2 Hz), 3.17 (dd, 1H, J=17.2, 4.3 Hz), 2.40 (m, 1H), 1.78 (m, 1H), 1.85-1.10 (m, 7H) ppm; C₁₇H₁₈N₆ (MW, 306.37), LCMS (EI) m/e 307 (M⁺+H).

### Step 2: Crystallization of the Amorphous Ruxolitinib Free Base in Aqueous Isopropanol to Generate Crystalline Ruxolitinib Di-hydrate.

To a solution of amorphous ruxolitinib free base from Step 1, 9.32 g, 28.1 mmol) in isopropanol (IPA, 86 mL) was gradually charged water (214 mL) at ambient temperature. The resulting mixture was warmed to 55-65° C. to generate a clear solution. The resulted solution was then cooled to ambient temperature before crystalline ruxolitinib di-hydrate seeds (20.9 mg) were introduced into the solution. The crystallization mixture was then stirred at ambient temperature for 16 hours and solids were gradually crystallized out of solution. The solids were collected by filtration, washed with cold water (50 mL), and dried under vacuum to constant weight to afford (R)-3-(4-(7H-pyrrolo[2,3-d]pyrimidin-4-yl)-1H-pyrazol-1-yl)-3-cyclopentylpropanenitrile di-hydrate (9.03 g, 9.62 g theoretical, 93.9%) as a white to off-white crystalline powder. For crystalline ruxolitinib di-hydrate: >99.9% of chemical purity by HPLC; >99.9% of chiral purity by Chiral HPLC; 10.30% of water content by KF; ¹H NMR (500 MHz, DMSO-d₆) δ 12.09 (s, 1H), 8.79 (s, 1H), 8.69 (s, 1H), 8.38 (s, 1H), 7.59 (dd, J=3.7, 2.0 Hz, 1H), 6.99 (dd, J=3.7, 1.4 Hz, 1H), 4.54 (td, J=9.7, 4.0 Hz, 1H), 3.28 (dd, J=17.1, 9.7 Hz, 1H), 3.19 (dd, J=17.1, 4.0 Hz, 1H), 2.49-2.37 (m, 1H), 1.85-1.78 (m, 1H), 1.66-1.58 (m, 1H), 1.57-1.49 (m, 2H), 1.48-1.39 (m, 1H), 1.37-1.26 (m, 2H), 1.25-1.16 (m, 1H); ¹³C NMR (DMSO-d₆, 126 MHz) δ 152.60, 151.42, 150.39, 139.71, 131.47, 127.17, 121.03, 118.63, 113.29, 100.24, 62.98, 44.80, 29.57, 29.53, 25.41, 24.80, 22.99; C₁₇H₂₂N₆O₂ (MW, 342.40 for ruxolitinib di-hydrate) and C₁₇H₁₈N₆ (MW, 306.37 for ruxolitinib free base), LCMS (EI) m/e 307 (M⁺+H for ruxolitinib free base).

### Alternative Step 2: Preparation of Crystalline Ruxolitinib Di-hydrate Without Seeding.

To a 40 mL sintillation vial with stir bar was charged (R)-3-(4-(7H-pyrrolo[2,3-d]pyrimidin-4-yl)-1H-pyrazol-1-yl)-3-cyclopentylpropanenitrile (ruxolitinib free base, 1.13 g, 3.61 mmol) and IPA (7.15 mL, 93 mmol) at ambient temperature. The mixture was warmed in a 60° C. water bath to partially dissolve the foam. Water (16.0 mL, 888 mmol) was then introduced and the resulting mixture was warmed in a 60° C. water bath until completely dissolution was obtained. The solution was then cooled to ambient temperature (18.2° C.) and agitated at ambient temperature. After stirring for 6 hours at ambient temperature, the solution became turbid. Additional amount of water (1.85 mL, 103 mmol) was charged and the resulting mixture was stirred at ambient temperature for additional 17 hours. Solids were collected by filtration and the wet cake was dried under house vacuum by pulling air through the filter cake. The desired product, (R)-3-(4-(7H-pyrrolo[2,3-d]pyrimidin-4-yl)-1H-pyrazol-1-yl)-3-cyclopentylpropanenitrile dihydrate (ruxolitinib dihydrate, 0.913 g, 1.236 g theoretical, 74% yield), was obtained as off-white to light pink crystalline powder, which is identical in every comparable aspect including XRPD and DSC to the crystalline ruxolitinib dihydrate made with seeding.

### Example 2. Preparation of Crystalline Ruxolitinib Di-hydrate From In-Situ Generated Ruxolitinib Free Base

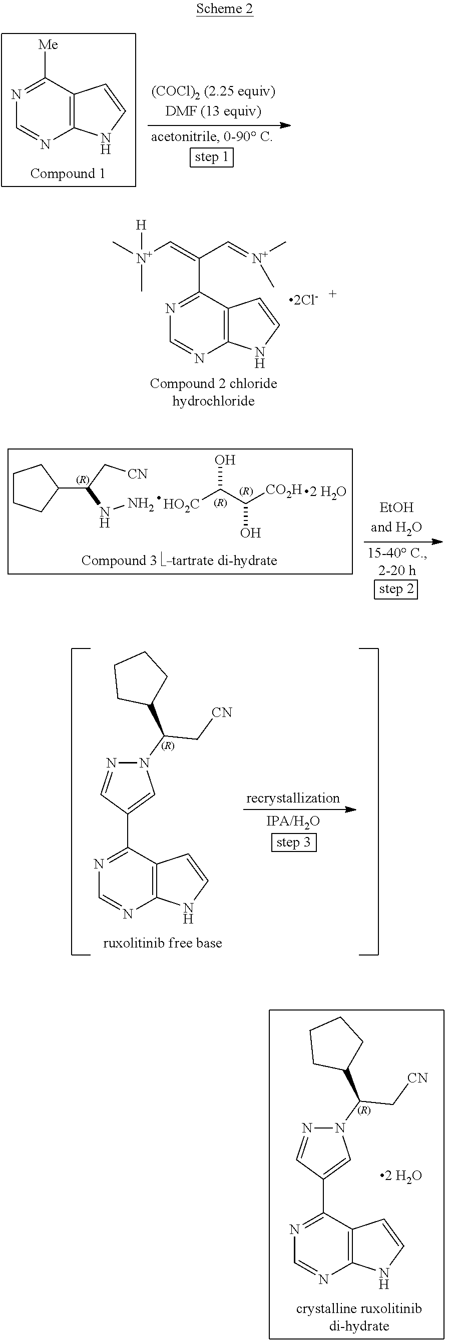

### Step 1: Preparation of (E)-N-(3-(Dimethylamino)-2-(7H-pyrrolo[2,3-d]pyrimidin-4-yl)allylidene)-N-methylmethanaminium Chloride Hydrochloride.

A solution of oxalyl chloride (21.88 g, 15.1 mL, 169 mmol, 2.25 equiv) in anhydrous acetonitrile (65 mL) was cooled to 0-5° C. in an ice bath. Anhydrous DMF (70.8 g, 75.0 mL, 969 mmol, 12.9 equiv) was added dropwise into the solution to form the corresponding Vilsmeier reagent. During addition of DMF, the internal temperature was controlled to below 10° C. The ice batch was removed and the reaction mixture was gradually warmed to ambient temperature over 40 minutes. Methyl-7H-pyrrolo[2,3-d]pyrimidine (Compound 1, 10.0 g, 75.1 mmol) was charged into the in-situ generated Vilsmeier reagent as a solid in one portion at ambient temperature, and the resulting slurry was agitated at ambient temperature for 5-10 minutes to ensure complete mixing before being warmed to 85-90° C. The reaction mixture was agitated at 85-90° C. for one hour before being gradually cooled to ambient temperature. Anhydrous acetone (CH₃COCH₃, 100 mL) was charged and the resulting slurry was agitated at ambient temperature for two hours followed by at 0-5° C. for two hours. The solids were collected by filtration, washed with a one to one mixture of acetone and MTBE (2×100 mL), and dried under vacuum to constant weight to afford the desired product, (E)-N-(3-(Dimethylamino)-2-(7H-pyrrolo[2,3 -d]pyrimidin-4-yl)allylidene)-N-methylmethanaminium chloride hydrochloride (Compound 2 chloride hydrochloride, 22.0 g, 23.72 g theoretical, 98.9% by HPLC area %, 97.6 wt %, 92.7% yield), as a yellow to brown crystalline solid which contained <1% of DMF and acetonitrile and 1-2% of water and was used in the subsequent reaction without further purification. For Compound 2 chloride hydrochloride: ¹H NMR (500 MHz, DMSO-d₆) δ 13.65 (s, 1H), 8.99 (s, 1H), 8.48 (s, 2H), 7.99-7.94 (m, 1H), 6.84 (dd, J=3.6, 1.6 Hz, 1H), 3.48 (s, 6H), 2.82 (s, 6H) ppm; ¹³C NMR (DMSO-d₆, 125 MHz) δ 163.8, 151.3, 147.6, 145.0, 132.1, 117.5, 102.9, 91.6, 48.9, 42.1 ppm; C₁₃H₁₉Cl₂N₅(MW, 279.77 for Compound 2 chloride hydrochloride and 244.32 for Compound 2 without anion) LCMS (EI) m/e 244.2 (M⁺, base peak). Note that the Compound 2 chloride hydrochloride is the same as Crystalline Form I of Compound 2b in WO 2022/040180.

Step 2: Preparation of (R)-3-(4-(7H-Pyrrolo[2,3-d]pyrimidin-4-yl)-111-pyrazol-1-yl)-3-cyclopentylpropanenitrile (Ruxolitinib Free Base).

A solution of (E)-N -(3-(dimethylamino)-2-(7H -pyrrolo[2,3-d]pyrimidin-4-yl)allylidene)-N-methylmethanaminium chloride hydrochloride (Compound 2 chloride hydrochloride, 10.0 g, 31.6 mmol) in water (10.0 mL) was treated with an 30% aqueous solution of NaOH at 0-5° C. to pH 7-8. To the resulting aqueous solution was added charcoal (2.0 g) and the mixture was agitated at ambient temperature for 2-4 hours. The charcoal was removed by filtration through a Celite bed and the wet charcoal cake was washed with water (15 mL). The resulting aqueous solution, which contained (E)-N-(3-(dimethylamino)-2-(7H-pyrrolo[2,3-d]pyrimidin-4-yl)allylidene)-N-methylmethanaminium chloride (Compound 2 chloride), was then treated with ethanol (30 mL) and (R)-3-cyclopentyl-3-hydrazinylpropanenitrile L-tartaric acid salt di-hydrate (Compound 3 L-tartrate di-hydrate, 11.21 g, 33.0 mmol, 1.04 equiv) at ambient temperature. The resulting mixture was then agitated at ambient temperature for 16 hours. When the reaction was complete, the reaction mixture was filtered to remove the solids (L-tartaric acid). The cake was washed with ethanol (2×10 mL). The filtrate and the wash solution were combined and the combined solution was concentrated under a reduced pressure at 40-50° C. to remove most of the ethanol. To the residue was then added H₂ O (30 mL) and dichloromethane (DCM, 50 mL) and the mixture was treated with a 30% aqueous sodium hydroxide (NaOH) solution to adjust the pH to around 10. The two layers were separated, and the aqueous layer was extracted with DCM (30 mL). The combined organic extracts were filtered through a Celite bed and the Celite bed was washed with DCM (10 mL). The solution of ruxolitinib free base in DCM was then utilized directly in the subsequent process step to generate crystalline ruxolitinib di-hydrate.

Step 3: Preparation of Crystalline (R)-3-(4-(7H-Pyrrolo[2,3-d]pyrimidin-4-yl)-111-pyrazol-1-yl)-3-cyclopentylpropanenitrile Di-hydrate (Crystalline Ruxolitinib Di-Hydrate).

A solution of ruxolitinib free base in DCM was concentrated under the reduced pressure to remove most of DCM. Isopropanol (IPA, 50 mL) was then charged to the residue and the resulting solution was further concentrated under the reduced pressure. Additional IPA (50 mL) was charged to the concentrated solution to completely switch the solvent to IPA. The resulting solution of (R)-3 -(4-(7H-pyrrolo[2,3-d]pyrimidin-4-yl)-1H-pyrazol-1-yl)-3-cyclopentylpropanenitrile (ruxolitinib free base) in IPA was then cooled to ambient temperature before water (208 mL) was gradually charged into the IPA solution at ambient temperature with stirring. Once water addition was complete, the resulting mixture was warmed to 55-65° C. to generate a clear solution. The resulting solution was then cooled to ambient temperature before crystalline ruxolitinib di-hydrate seeds (32 mg) (isolated from the approach set forth above) were introduced into the solution. The crystallization mixture was then stirred at ambient temperature for 16 hours and solids were gradually crystallized out of solution. The solids were collected by filtration, washed with a mixture of IPA and water (20% IPA in water, 25 mL), and dried under vacuum to constant weight to afford ruxolitinib di-hydrate (6.76 g, 10.82 g theoretical, 62.5%) as a white to off-white crystalline powder, which is identical in every comparable aspect to the material obtained in Example 1.

### Physicochemical Properties of the Crystalline Ruxolitinib Di-hydrate

The structure of ruxolitinib di-hydrate was determined by the single crystal x-ray analysis. See Example 3. The physicochemical properties of the crystalline ruxolitinib di-hydrate were characterized by X-Ray Powder Diffraction (XRPD), Differential Scanning calorimetry (DSC), Thermogravimetric Analysis (TGA), and Dynamic Vapor Sorption (DVS). See Examples 4-7.

### Example 3. Single Crystal X-Ray Analysis of Ruxolitinib Di-hydrate

A colorless block crystal having approximate dimensions of 0.260×0.190×0.120 mm³, was mounted on a polymer loop in random orientation. Preliminary examination and data collection were performed on a Bruker AXS D8 Quest diffractometer, equipped with a copper anode microsource sealed X-ray tube (Cu Kαλ=1.54178 Å) and a PhotonIII_C14 charge-integrating and photon counting pixel array detector.

Cell constants and an orientation matrix for data collection were obtained from least-squares refinement using the setting angles of 9104 reflections in the range 3.4603°<θ<78.3810°. The data were collected to a maximum diffraction angle (2θ) of 159.822° at room temperature.

The crystal system is orthorhombic and the space group is P212121. The cell parameters and calculated volume are: a=9.9731(11) Å, b=15.1765(17) Å, c=23.649(3) Å, α=90°, β=90°, γ=90°, V=3579.4(7) Å3. The formula weight is 342.40 g mol⁻¹ with Z=8, resulting in a calculated density of 1.271 g cm⁻³.

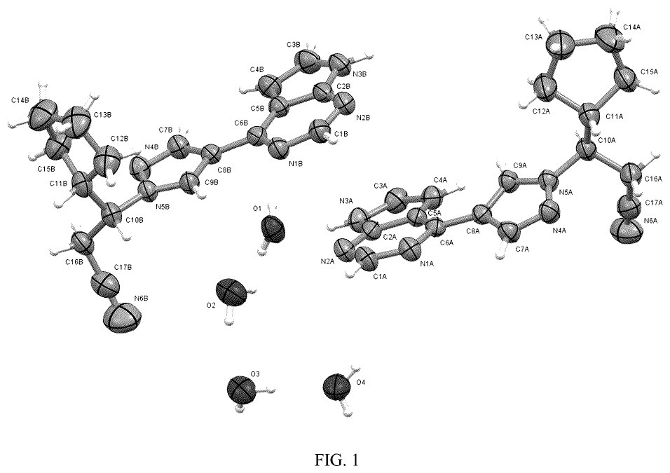

The single crystal structure of ruxolitinib di-hydrate was determined. The structure of ruxolitinib di-hydrate was determined to be a hydrated crystal form, composed of two ruxolitinib molecules and four water molecules in the asymmetric unit, as shown in FIG. 1.

### Example 4A. X-Ray Powder Diffraction (XRPD) Analysis of Crystalline Ruxolitinib Di-hydrate

X-Ray Powder Diffraction (XRPD) was obtained from a Bruker D8 Advance ECO X-ray Powder Diffractometer (XRPD) instrument. The general experimental procedures for XRPD were: (1) X-ray radiation from copper at 1.5418 Å and LYNXEYE™ detector with a slit of 0.6 mm; (2) X-ray power at 40 kV, 25 mA; and (3) the sample powder was dispersed on a zero-background sample holder. The general measurement conditions for XRPD were: Start Angle 3 degrees; Stop Angle 30 degrees; Sampling 0.015 degrees; and Scan speed 2 degree/min.

XRPD analysis is one means of determining crystallinity. Here, XRPD analysis of a sample of ruxolitinib di-hydrate showed that it was a crystalline solid (FIG. 2). The XRPD pattern is consistent with the calculated XRPD pattern from the single crystal structure. The

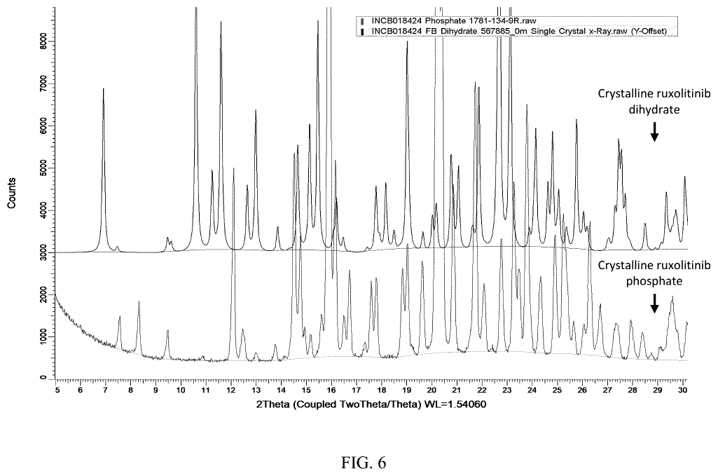

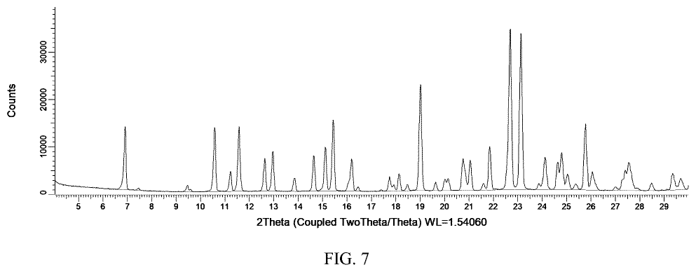

XRPD peak data corresponding to FIG. 2 are provided in Table 1. FIG. 6 shows a comparison of the XRPD pattern of ruxolitinib di-hydrate with the XRPD pattern of ruxolitinib phosphate. Another batch of the ruxolitinib di-hydrate also shows that the product is crystalline, see Table 2 and FIG. 7.

TABLE 1
XRPD Data for a Representative Sample
of Crystalline Ruxolitinib Di-hydrate

| 2-Theta (°) | H % |
|---|---|
| 6.9 | 31.7 |
| 7.4 | 1.1 |
| 9.4 | 3.3 |
| 10.6 | 44.0 |
| 11.2 | 13.0 |
| 11.6 | 39.4 |
| 12.6 | 15.8 |
| 12.9 | 24.7 |
| 13.8 | 7.0 |
| 14.6 | 22.5 |
| 15.1 | 28.6 |
| 15.4 | 48.0 |
| 16.2 | 15.1 |
| 16.4 | 3.1 |
| 17.4 | 0.8 |
| 17.7 | 9.8 |
| 18.1 | 11.5 |
| 18.4 | 3.6 |
| 19.0 | 59.2 |
| 19.6 | 4.4 |
| 20.1 | 8.3 |
| 20.7 | 21.3 |
| 21.0 | 19.4 |
| 21.6 | 4.3 |
| 21.8 | 30.0 |
| 22.7 | 100 |
| 23.1 | 86.6 |
| 23.9 | 3.5 |
| 24.1 | 21.3 |
| 24.6 | 16.6 |
| 24.8 | 26.6 |
| 25.0 | 10.5 |
| 25.3 | 4.5 |
| 25.7 | 34.1 |
| 26.0 | 9.5 |
| 27.0 | 2.5 |
| 27.4 | 15.3 |
| 27.5 | 20.7 |
| 27.9 | 1.6 |
| 28.5 | 5.0 |
| 28.9 | 0.3 |
| 29.3 | 11.7 |
| 29.7 | 8.0 |

TABLE 2
XRPD Data for Another Representative Sample
of Crystalline Ruxolitinib Di-hydrate

| 2-Theta (°) | H % |
|---|---|
| 6.9 | 38.9 |
| 7.5 | 1.4 |
| 9.5 | 4.0 |
| 10.6 | 39.6 |
| 11.2 | 12.3 |
| 11.6 | 39.7 |
| 12.6 | 20.2 |
| 13.0 | 24.7 |
| 13.9 | 8.3 |
| 14.6 | 21.9 |
| 15.1 | 27.1 |
| 15.4 | 44.1 |
| 16.2 | 19.9 |
| 16.4 | 2.5 |
| 17.4 | 0.8 |
| 17.7 | 9.0 |
| 17.9 | 3.9 |
| 18.1 | 10.7 |
| 18.5 | 3.8 |
| 19.0 | 65.9 |
| 19.6 | 5.2 |
| 20.1 | 7.3 |
| 20.8 | 19.8 |
| 21.1 | 18.8 |
| 21.6 | 4.2 |
| 21.8 | 27.0 |
| 22.1 | 0.6 |
| 22.7 | 100 |
| 23.1 | 96.9 |
| 23.9 | 3.7 |
| 24.1 | 20.0 |
| 24.6 | 17.0 |
| 24.8 | 22.9 |
| 25.0 | 9.7 |
| 25.4 | 3.5 |
| 25.8 | 40.8 |
| 26.1 | 11.3 |
| 27.0 | 2.1 |
| 27.4 | 12.6 |
| 27.6 | 17.4 |
| 27.9 | 1.4 |
| 28.5 | 4.8 |
| 28.9 | 0.4 |
| 29.1 | 0.5 |
| 29.3 | 10.2 |
| 29.7 | 7.0 |

### Example 4B. XRPD Characterization (Method A) of Additional Representative Sample of Ruxolitinib Di-hydrate

Additional representative samples of ruxolitinib di-hydrate were analyzed.

### Transmission Geometry

XRPD patterns were collected with a PANalytical X'Pert PRO MPD or a PANalytical Empyrean diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640f) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 5.5.

Under most circumstances, peaks within the range of up to about 30° 2Θ were selected. Rounding algorithms were used to round each peak to the nearest 0.1° or 0.01° 2Θ, depending upon the instrument used to collect the data and/or the inherent peak resolution. The location of the peaks along the x-axis (° 2Θ) in both the figures and the tables were determined using proprietary software and rounded to one or two significant figures after the decimal point based upon the above criteria. Peak position variabilities are given to within ±0.2° 2Θ based upon recommendations outlined in the USP discussion of variability in x-ray powder diffraction. The accuracy and precision associated with any particular measurement reported herein has not been determined. Moreover, third party measurements on independently prepared samples on different instruments may lead to variability which is greater than ±0.2° 2Θ. For d-space listings, the wavelength used to calculate d-spacings was 1.5405929 Å, the Cu—K_{α1} wavelength. Variability associated with d-spacing estimates was calculated from the USP recommendation, at each d-spacing, and provided in the respective data tables.

Per USP guidelines, variable hydrates and solvates may display peak variances greater than 0.2° 2Θ and therefore peak variances of 0.2° 2Θ are not applicable to these materials.

For samples with only one XRPD pattern and no other means to evaluate whether the sample provides a good approximation of the powder average, peak tables contain data identified only as “Prominent Peaks”. These peaks are a subset of the entire observed peak list. Prominent peaks are selected from observed peaks by identifying preferably non-overlapping, low-angle peaks, with strong intensity.

If multiple diffraction patterns are available, then assessments of particle statistics (PS) and/or preferred orientation (PO) are possible. Reproducibility among XRPD patterns from multiple samples analyzed on a single diffractometer indicates that the particle statistics are adequate. Consistency of relative intensity among XRPD patterns from multiple diffractometers indicates good orientation statistics. Alternatively, the observed XRPD pattern may be compared with a calculated XRPD pattern based upon a single crystal structure, if available. Two-dimensional scattering patterns using area detectors can also be used to evaluate PS/PO. If the effects of both PS and PO are determined to be negligible, then the XRPD pattern is representative of the powder average intensity for the sample and prominent peaks may be identified as “Representative Peaks”. In general, the more data collected to determine Representative Peaks, the more confident one can be of the classification of those peaks. “Characteristic peaks”, to the extent they exist, are a subset of Representative Peaks and are used to differentiate one crystalline polymorph from another crystalline polymorph (polymorphs being crystalline forms having the same chemical composition). Characteristic peaks are determined by evaluating which representative peaks, if any, are present in one crystalline polymorph of a compound against all other known crystalline polymorphs of that compound to within ±0.2° 2Θ. Not all crystalline polymorphs of a compound necessarily have at least one characteristic peak.

FIG. 9 shows an XRPD pattern of crystalline ruxolitinib di-hydrate, Table 3A shows the observed peaks, and Table 3B shows the prominent peaks.

TABLE 3A
Observed Peaks for Ruxolitinib Di-hydrate

| 2-Theta (°) | d space (Å) | H (%) |
|---|---|---|
| 6.91 ± 0.20 | 12.778 ± 0.369 | 44 |
| 7.46 ± 0.20 | 11.847 ± 0.317 | 4 |
| 9.47 ± 0.20 | 9.332 ± 0.197 | 6 |
| 9.60 ± 0.20 | 9.201 ± 0.191 | 5 |
| 10.60 ± 0.20 | 8.339 ± 0.157 | 88 |
| 11.24 ± 0.20 | 7.864 ± 0.139 | 25 |
| 11.59 ± 0.20 | 7.629 ± 0.131 | 68 |
| 12.64 ± 0.20 | 6.997 ± 0.110 | 19 |
| 12.98 ± 0.20 | 6.814 ± 0.105 | 40 |
| 13.86 ± 0.20 | 6.384 ± 0.092 | 9 |
| 14.66 ± 0.20 | 6.037 ± 0.082 | 31 |
| 15.14 ± 0.20 | 5.849 ± 0.077 | 36 |
| 15.46 ± 0.20 | 5.727 ± 0.074 | 65 |
| 16.09 ± 0.20 | 5.505 ± 0.068 | 8 |
| 16.21 ± 0.20 | 5.465 ± 0.067 | 16 |
| 16.47 ± 0.20 | 5.377 ± 0.065 | 5 |
| 17.43 ± 0.20 | 5.084 ± 0.058 | 3 |
| 17.77 ± 0.20 | 4.988 ± 0.056 | 21 |
| 17.95 ± 0.20 | 4.938 ± 0.055 | 5 |
| 18.16 ± 0.20 | 4.880 ± 0.053 | 22 |
| 18.50 ± 0.20 | 4.793 ± 0.051 | 6 |
| 19.03 ± 0.20 | 4.660 ± 0.049 | 56 |
| 19.65 ± 0.20 | 4.515 ± 0.046 | 6 |
| 20.04 ± 0.20 | 4.426 ± 0.044 | 11 |
| 20.17 ± 0.20 | 4.398 ± 0.043 | 15 |
| 20.78 ± 0.20 | 4.271 ± 0.041 | 26 |
| 21.08 ± 0.20 | 4.212 ± 0.040 | 22 |
| 21.63 ± 0.20 | 4.106 ± 0.038 | 8 |
| 21.87 ± 0.20 | 4.060 ± 0.037 | 44 |
| 22.70 ± 0.20 | 3.914 ± 0.034 | 100 |
| 23.14 ± 0.20 | 3.840 ± 0.033 | 78 |
| 23.90 ± 0.20 | 3.720 ± 0.031 | 7 |
| 24.14 ± 0.20 | 3.684 ± 0.030 | 32 |
| 24.67 ± 0.20 | 3.606 ± 0.029 | 17 |
| 24.82 ± 0.20 | 3.584 ± 0.028 | 28 |
| 25.07 ± 0.20 | 3.549 ± 0.028 | 16 |
| 25.39 ± 0.20 | 3.505 ± 0.027 | 7 |
| 25.79 ± 0.20 | 3.452 ± 0.026 | 32 |
| 26.08 ± 0.20 | 3.414 ± 0.026 | 11 |
| 27.04 ± 0.20 | 3.294 ± 0.024 | 6 |
| 27.48 ± 0.20 | 3.243 ± 0.023 | 28 |
| 28.51 ± 0.20 | 3.128 ± 0.021 | 9 |
| 29.38 ± 0.20 | 3.038 ± 0.020 | 15 |
| 29.73 ± 0.20 | 3.003 ± 0.020 | 14 |
| 30.12 ± 0.20 | 2.965 ± 0.019 | 19 |
| 30.56 ± 0.20 | 2.923 ± 0.019 | 4 |
| 30.82 ± 0.20 | 2.899 ± 0.018 | 5 |
| 31.05 ± 0.20 | 2.878 ± 0.018 | 4 |
| 31.52 ± 0.20 | 2.836 ± 0.018 | 8 |

TABLE 3B
Representative Peaks for Ruxolitinib Di-hydrate

| | | Intensity |
|---|---|---|
| °2θ | d space (Å) | (%) |
| 6.91 ± 0.20 | 12.778 ± 0.369 | 44 |
| 10.60 ± 0.20 | 8.339 ± 0.157 | 88 |
| 11.24 ± 0.20 | 7.864 ± 0.139 | 25 |
| 11.59 ± 0.20 | 7.629 ± 0.131 | 68 |
| 12.64 ± 0.20 | 6.997 ± 0.110 | 19 |
| 12.98 ± 0.20 | 6.814 ± 0.105 | 40 |
| 14.66 ± 0.20 | 6.037 ± 0.082 | 31 |
| 15.14 ± 0.20 | 5.849 ± 0.077 | 36 |
| 15.46 ± 0.20 | 5.727 ± 0.074 | 65 |
| 17.77 ± 0.20 | 4.988 ± 0.056 | 21 |
| 18.16 ± 0.20 | 4.880 ± 0.053 | 22 |
| 19.03 ± 0.20 | 4.660 ± 0.049 | 56 |
| 20.78 ± 0.20 | 4.271 ± 0.041 | 26 |
| 21.08 ± 0.20 | 4.212 ± 0.040 | 22 |
| 21.87 ± 0.20 | 4.060 ± 0.037 | 44 |
| 22.70 ± 0.20 | 3.914 ± 0.034 | 100 |
| 23.14 ± 0.20 | 3.840 ± 0.033 | 78 |
| 24.14 ± 0.20 | 3.684 ± 0.030 | 32 |
| 24.82 ± 0.20 | 3.584 ± 0.028 | 28 |
| 25.79 ± 0.20 | 3.452 ± 0.026 | 32 |
| 27.48 ± 0.20 | 3.243 ± 0.023 | 28 |

### Example 5. Differential Scanning Calorimetry (DSC) Analysis on Crystalline Ruxolitinib Di-hydrate

DSC was obtained from a TA Instruments Differential Scanning calorimetry, Discovery DSC2500 with autosampler. The DSC instrument conditions were as follows: 20-300° C. at 10° C./min; Tzero aluminum sample pan and lid; and nitrogen gas flow at 50 mL/min.

The DSC thermogram of a representative sample of crystalline ruxolitinib di-hydrate is shown in FIG. 3. The DSC thermogram revealed one endothermal event at an onset temperature of 60.5° C. with a peak temperature of 66.7° C. which corresponds to the dehydration process. In another DSC thermogram of a representative sample of crystalline ruxolitinib di-hydrate had one endothermal event at an onset temperature of 59.5° C. In yet another DSC thermogram of a representative sample of crystalline ruxolitinib di-hydrate had one endothermal event with a peak temperature of 69.1° C.

A separate DSC thermogram was performed for the material. For this experiment, DSC was performed using a Mettler-Toledo DSC3+ differential scanning calorimeter (Method B). A tau lag adjustment was performed with indium, tin, and zinc. The temperature and enthalpy were adjusted with octane, phenyl salicylate, indium, tin and zinc. The adjustment was then verified with octane, phenyl salicylate, indium, tin, and zinc. The sample was placed into a hermetically sealed aluminum DSC pan, the weight was accurately recorded, and the sample was inserted into the DSC cell. A weighed aluminum pan configured as the sample pan was placed on the reference side of the cell. The pan lid was pierced prior to sample analysis. The samples were analyzed from −30° C. to 250° C. at 10° C./min.

The DSC thermogram of a representative sample of crystalline ruxolitinib di-hydrate is shown in FIG. 10. A broad endothermic event was observed at an onset temperature of 68° C. and a peak temperature of 72° C. with a secondary endotherm with a peak maximum of 110° C.

### Example 6A. Thermogravimetric Analysis (TGA) Analysis on Crystalline Ruxolitinib Di-Hydrate

TGA was obtained from a TA Instruments Thermogravimetric Analyzer, Discovery TGA5500 with autosampler. The general experimental conditions for TGA were: ramp from 25° C. to 300° C. at 10° C./min; nitrogen purge gas flow at 25 mL/min; platinum sample holder.

The TGA thermogram of a representative sample of crystalline ruxolitinib di-hydrate is shown in FIG. 4. Weight loss of 10.4% was observed below 100° C. due to dehydration. The resulting anhydrous free base decomposed at above 200° C.

The representative sample of crystalline ruxolitinib di-hydrate lost all hydrate from 30° C. to 80° C. to become an amorphous solid. The amorphous solid remained an amorphous solid after adding one drop of water. The amorphous solid became crystalline di-hydrate after adding two drops of water after one day.

### Example 6B. Additional Thermogravimetric Analysis on Crystalline Ruxolitinib Di-hydrate

An additional TGA thermogram was performed for the material. Thermogravimetric analyses were performed using a Mettler-Toledo TGA/DSC3+ analyzer (Method C). Temperature and enthalpy adjustments were performed using indium, tin, zinc, and phenyl salicylate, and then verified with indium. The balance was verified with calcium oxalate. The sample was placed in an aluminum pan. The pan was hermetically sealed, the lid pierced, and the pan was then inserted into the TG furnace. A weighed aluminum pan configured as the sample pan was placed on the reference platform. The furnace was heated under nitrogen. Samples were analyzed from ambient to 350° C. at 10° C./min. Thermogravimetric analyses typically experience a period of equilibration at the start of each analysis, indicated by bracket on the thermograms. The starting temperature for relevant weight loss calculations is selected at a point beyond this region (typically above 35° C.) for accuracy.

The TGA thermogram of a representative sample of crystalline ruxolitinib di-hydrate is shown in FIG. 11. TGA analysis indicated a 1.7% weight loss up to 90° C. followed by an additional 8.2% up to 178° C. A 10.5% (9.9% observed) weight loss would be consistent with the dihydrate.

### Example 7A. Dynamic Vapor Sorption (DVS) Analysis on Crystalline Ruxolitinib Di-hydrate

The DVS experiment was performed on a VTI-SA+Vapor Sorption Analyzer from TA Instruments. A representative sample of crystalline ruxolitinib di-hydrate was first pre-dried at 60° C. under dry N₂ (0% RH) for 1 h. Then the humidity was cycled from 5% RH to 95% RH (adsorption) and back to 5% RH (desorption) with 5% RH interval at a constant temperature of 25° C. The equilibration criteria were 0.010 wt % in 5 minutes with a maximum equilibration time of 180 minutes per step.

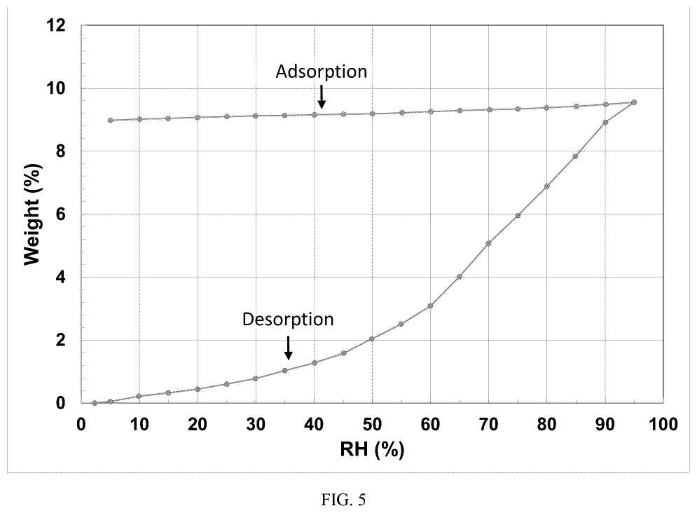

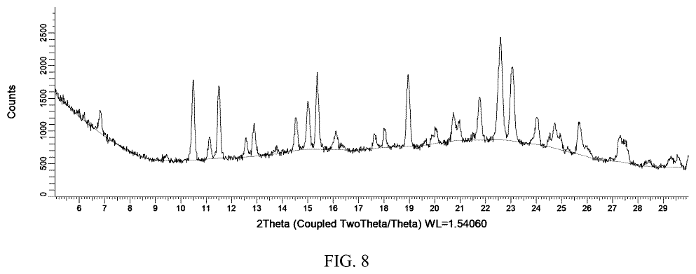

In the pre-dried step at 60° C., a representative sample of crystalline ruxolitinib di-hydrate was dehydrated to form amorphous ruxolitinib free base. Then it re-adsorbed water up to 9.6% at 95% RH and kept relatively stable down to 5% RH. The XRPD analysis showed that the resulted solid is crystalline ruxolitinib di-hydrate after DVS, see FIG. 8 and Table 4. The DVS experimental results performed on this representative sample of crystalline ruxolitinib di-hydrate are shown in FIG. 5.

TABLE 4
XRPD Data for Crystalline Ruxolitinib Di-Hydrate after DVS

| 2-Theta (°) | H % |
|---|---|
| 6.8 | 22.1 |
| 9.4 | 5.3 |
| 10.5 | 78.0 |
| 11.1 | 21.0 |
| 11.5 | 70.6 |
| 12.6 | 18.7 |
| 12.9 | 31.6 |
| 13.8 | 8.2 |
| 14.5 | 31.7 |
| 15.0 | 46.3 |
| 15.4 | 73.9 |
| 16.1 | 18.1 |
| 17.6 | 14.2 |
| 18.0 | 18.6 |
| 19.0 | 70.0 |
| 19.6 | 4.5 |
| 20.0 | 12.8 |
| 20.7 | 26.3 |
| 20.9 | 17.4 |
| 21.8 | 41.9 |
| 22.6 | 100 |
| 23.1 | 71.1 |
| 24.0 | 26.7 |
| 24.5 | 13.2 |
| 24.7 | 24.4 |
| 25.0 | 14.5 |
| 25.2 | 5.4 |
| 25.7 | 30.1 |
| 26.0 | 9.3 |
| 27.3 | 25.2 |
| 27.5 | 22.2 |
| 28.4 | 5.2 |
| 29.3 | 10.6 |
| 29.6 | 11.5 |

### Example 7B. Additional DVS Analysis

Additional DVS on crystalline ruxolitinib di-hydrate was collected on a Surface Measurement System DVS Intrinsic instrument (Method D). The samples were not dried prior to analysis. Sorption and desorption data were collected from 5% to 95% RH in 10% RH increments under a nitrogen purge. The equilibrium criteria used for the analyses were 0.001 dm/dt weight change in 5 minutes with a minimum step time of 30 minutes and maximum equilibration time of 180 minutes with a 3 minute data logging interval. Data were not corrected for the initial moisture content of the sample.

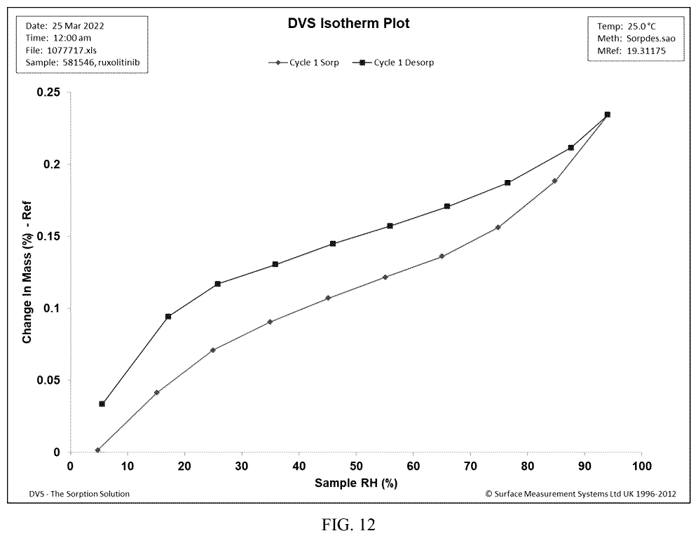

DVS showed that ruxolitinib di-hydrate gained 0.23% weight from 5 to 95% relative humidity and lost 0.20% upon desorption during the DVS analysis (FIG. 12 and Table 5).

TABLE 5
DVS Data of Ruxolitinib Di-hydrate

| | | | | Desorp | |
|---|---|---|---|---|---|
| Target | Sample | Sorp Mass | Sample | Mass | |
| RH | RH | Change | RH | Change | |
| (%) | (%) | (%) | (%) | (%) | Hysteresis |
| 5.0 | 4.8 | 0.0013 | 5.6 | 0.0334 | |
| 15.0 | 15.1 | 0.0412 | 17.1 | 0.0940 | 0.0528 |
| 25.0 | 25.0 | 0.0707 | 25.8 | 0.1168 | 0.0461 |
| 35.0 | 35.0 | 0.0904 | 35.9 | 0.1302 | 0.0399 |
| 45.0 | 45.1 | 0.1069 | 46.0 | 0.1447 | 0.0378 |
| 55.0 | 55.1 | 0.1214 | 56.0 | 0.1572 | 0.0357 |
| 65.0 | 65.1 | 0.1359 | 66.0 | 0.1706 | 0.0347 |
| 75.0 | 74.9 | 0.1561 | 76.6 | 0.1872 | 0.0311 |
| 85.0 | 84.8 | 0.1882 | 87.6 | 0.2115 | 0.0233 |
| 95.0 | 94.0 | 0.2343 | 94.0 | 0.2343 | |

### Example 8. Hot Stage Microscopy of Crystalline Ruxolitinib Di-hydrate

A representative sample of crystalline ruxolitinib di-hydrate was heated at a rate of 5° C./min starting from a temperature of 25° C. The crystals showed no significant change at temperatures at and below 60° C. The crystals became darker from 62° C. to 70° C. which corresponded to the dehydration process. Melting was observed at approximately 75° C. and was completed at 80° C. No crystallization was observed after cooling to 25° C.

### Example 9. Aqueous Solubility of Ruxolitinib Di-hydrate

The solubility of representative samples of crystalline ruxolitinib di-hydrate were measured under different aqueous solutions (Table 6) at 20-25° C. The results are summarized in Table 6.

TABLE 6
Aqueous Solubility Data for Ruxolitinib Di-hydrate

| Media (20-25° C.) | Solubility (mg/mL) |
|---|---|
| 0.1N HCl | >10.7003 |
| SGF (pH 1.6) | >10.9974 |
| pH 2.0 (HCl) | 3.4711 |
| pH 3.0 (KHP) | 1.5708 |
| pH 4.0 (KPH) | 0.3620 |
| pH 4.0 (Acetate) | 0.1861 |
| pH 5.0 (KHP) | 0.1471 |
| pH 5.0 (Acetate) | 0.2960 |
| pH 5.5 (KHP) | 0.1066 |
| pH 5.5 (Acetate) | 0.0935 |
| pH 6.0 (PBS) | 0.0174 |
| pH 6.5 (PBS) | 0.1061 |
| pH 7.0 (PBS) | 0.1011 |
| pH 7.4 (PBS) | 0.1000 |
| pH 8.5 (Borate)* | 0.1116 |
| FeSSIF (pH 5.0) | 0.4194 |
| FeSSIF (pH 6.5) | 0.1116 |
| Saline (pH 5.5) | 0.1047 |
| Water | 0.1106 |

*Borate buffer prepared from boric acid and potassium chloride. The buffer pH is adjusted by sodium hydroxide.

### Example 10. Solubility of Crystalline Ruxolitinib Di-hydrate in Organic Solvents

The solubility of representative samples of crystalline ruxolitinib di-hydrate was measured under different organic solvents (Table 7). The results are summarized in Table 7. In polar solvents, ruxolitinib di-hydrate lost crystalline water. The resulting ruxolitinib free base readily dissolved in the organic solvents.

TABLE 7
Solubility Data for Ruxolitinib Di-hydrate in Organic Solvents

| Solvent | Solubility (mg/mL) |
|---|---|
| Methanol (MeOH) | >500 |
| Ethanol (EtOH) | >500 |
| Isopropanol (IPA) | >500 |
| Acetone | >500 |
| Ethyl Acetate | >200 |

### Example 11. Characterization of Single Crystals of Ruxolitinib Di-hydrate

### Preparation

Single crystals of ruxolitinib di-hydrate were prepared as follows. A vial was charged with 98.4 mg of amorphous ruxolitinib and contacted with 1 ml of ethyl acetate. The resulting solution was dried over magnesium sulfate. The sample was seeded with crystalline ruxolitinib di-hydrate followed by the addition of 1 ml of heptane resulting in oiling. The sample was contacted with 0.02 ml of water resulting in seed material dissolving. The sample was reseeded and 2 ml of heptane added. The sample was stored for 8 days at room temperature resulting in nucleation of hexagonal tablets.

### Data Collection

A colorless plate having approximate dimensions of 0.56×0.36×0.08 mm³, was mounted on a polymer loop in random orientation. Preliminary examination and data collection were performed on a Rigaku SuperNova diffractometer, equipped with a copper anode microfocus sealed X-ray tube (Cu Kαλ=1.54184 Å) and a Dectris Pilatus3 R 200K hybrid pixel array detector.

Cell constants and an orientation matrix for data collection were obtained from least-squares refinement using the setting angles of 12431 reflections in the range 4.7310°<θ<75.4160°. The space group was determined by the program CRYSALISPRO to be P2₁2₁2₁ (international tables no. 19).

Table 8 below provide the crystal data and data collection parameters.

TABLE 8
Crystal Data and Data Collection Parameters
for Ruxolitinib Dihydrate.

| | |
|---|---|
| Empirical formula | C₁₇H₂₂N₆O₂ |
| Formula weight (g mol⁻¹) | 342.40 |
| Temperature (K) | 299.90(10) |
| Wavelength (Å) | 1.54184 |
| Crystal system | orthorhombic |
| Space group | P2₁2₁2₁ |
| Unit cell parameters | |
| a = 9.96950(10) Å | α = 90° |
| b = 15.1563(2) Å | β = 90° |
| c = 23.6432(2) Å | γ = 90° |
| Unit cell volume (Å³) | 3572.51(7) |
| Cell formula units, Z | 8 |
| Calculated density (g cm⁻³) | 1.273 |
| Absorption coefficient (mm⁻¹) | 0.714 |
| F(000) | 1456 |
| Crystal size (mm³) | 0.56 × 0.36 × 0.08 |
| Reflections used for cell measurement | 12431 |
| θ range for cell measurement | 4.7310°-75.4160° |
| Total reflections collected | 17385 |
| Index ranges | −12 ≤ h ≤ 7; −18 ≤ k ≤ |
| | 18; −29 ≤ l ≤ |
| θ range for data collection | θₘᵢₙ = 3.464°, θₘₐₓ = 75.877° |
| Completeness to θₘₐₓ | 98.1% |
| Completeness to θ_{full} = 67.684° | 99.9% |
| Absorption correction | multi-scan |
| Transmission coefficient range | 0.758-1.000 |
| Refinement method | full matrix least-squares on F² |
| Independent reflections | 7190 [Rᵢₙₜ = 0.0167, R_{σ} = |
| | 0.0199] |
| Reflections [I > 2σ(I)] | 6861 |
| Reflections/restraints/parameters | 7190/0/491 |
| Goodness-of-fit on F² | S = 1.04 |
| Final residuals [I > 2σ(I)] | R = 0.0397, R_{w} = 0.1153 |
| Final residuals [all reflections] | R = 0.0413, R_{w} = 0.1168 |
| Largest diff. peak and hole (e Å⁻³) | 0.340, −0.215 |
| Max/mean shift/standard uncertainty | 0.001/0.000 |
| Absolute structure determination | Flack parameter: 0.18(6) |

### Calculated X-Ray Powder Diffraction (XRPD) Pattern

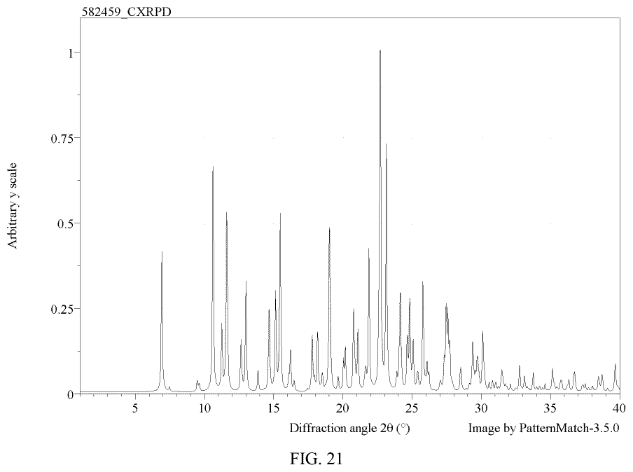

A calculated XRPD pattern was generated for Cu radiation using MERCURY and the atomic coordinates, space group, and unit cell parameters from the single crystal structure. See FIG. 21.

### Example 12. Relative Humidity Testing of Ruxolitinib Di-Hydrate

Ruxolitinib di-hydrate was held at select relative humidities to help determine range of physical stability (see table below). The d-hydrate was transferred to a vial, which was then uncapped and placed inside ajar, for a designated time period, containing a saturated aqueous salt solution:

| | Salt Utilized for RH |
|---|---|
| Humidity @ 25° C. | Jar |
| 11% RH | lithium chloride |
| 33% RH | magnesium chloride |
| 53% RH | magnesium nitrate |
| 58% RH | sodium bromide |
| 69% RH | potassium iodide |
| 98% RH | potassium sulfate |

The di-hydrate form was observed to be physically stable above 11% RH with conversion to an anhydrous crystalline free base form (Form I) at an undetermined value below 11% RH during the time tested. XRPD peak positions are consistent between XRPD patterns and are not representative of a variable system.

| Source | Condition | Duration | Result |
|---|---|---|---|
| Ruxolitinib Di- | 0% RH, N₂, RT | 1 d | Di-hydrate + Form I |
| hydrate | 11% RH, RT | 2 d | Di-hydrate |
| | | 14 d | Di-hydrate |
| | 33% RH, RT | 2 d | Di-hydrate |
| | 53% RH, RT | 2 d | Di-hydrate |
| | 98% RH, RT | 2 d | Di-hydrate |
| | | 4 d | Di-hydrate |

### Example 13. Water Slurry Experiments

Limited water activity slurries were conducted utilizing ruxolitinib di-hydrate (see table below). The effect of water activity (a_{w}) on the hydration state of the di-hydrate was investigated through competitive water activity trituration experiments (slurries) in various aqueous solvent mixtures. The resulting solid phase was characterized by XRPD. The experiments establish the physically stable form at various a w . Water activity is also related to relative humidity in that RH %=a_{w}×100. Therefore, it is possible to directly relate the stability of an anhydrous/hydrate system in slurry experiments to solid-state stability. Literature suggests that the slurry technique at controlled water activities provides an accurate method of rapidly predicting the physically stable form in anhydrous/hydrate systems. The method is particularly valuable when relatively slow kinetics of conversion in the solid state prevents reaching true equilibrium in a reasonable timeframe, since solvent-mediated transformation accelerates the conversion process.

The crystalline di-hydrate form was observed from all conditions tested.

| Source | Condition | Time | Result |
|---|---|---|---|
| Ruxolitinib | 0.68 a_{w} | 20 d | Di-hydrate |
| Di-hydrate | 50:50 DMSO/H₂O | | fine blades, slurry analysis |
| | 0.42 a_{w} | 20 d | Di-hydrate |
| | 70:30 DMSO/H₂O | | tablet like, slurry analysis |
| | 0.34 a_{w} | 20 d | Di-hydrate |
| | 75:25 DMSO/H₂O | | large blade, slurry analysis |

### Example 14. Dehydration of Ruxolitinib Di-hydrate

Dehydration of ruxolitinib di-hydrate results in the isolation of anhydrous crystalline ruxolitinib free base (Form I) (see table below). Form I is described in more detail in Example 15.

| Source | Technique¹ | Observation² | Result |
|---|---|---|---|
| Ruxolitinib | RT, vacuum oven, 1 d | — | Form I |
| Di-hydrate | | | disordered |
| | RT, vacuum oven, 1 d | — | Form I |
| | | | disordered |
| | 42° C. oven, 1 d | free flowing | Di- |
| | | | hydrate |
| | 50° C., vacuum oven, 1 d | — | Form I |
| | | | disordered |
| | P₂O₅ jar, RT, 4 d | — | Form I |
| | P₂O₅ jar, RT, 5 d | free flowing | Form I |
| | P₂O₅ jar, RT, 13 d | | conversion |
| | | | to Form |
| | | | I known |
| | 80° C./30 minutes | irregular, NB | amorphous |
| | 110° C./20 minutes | pink film, NB | amorphous |
| Ruxolitinib | P₂O₅ jar, RT, 21 d | opaque blades | Form I |
| Di-hydrate | | and chunks | disordered |
| crystals | | | |

¹Reported time, temperature, and humidity are approximate. RT = Room temperature.
²B = birefringence and NB = no birefringence upon observation by polarized light microscopy.

### Example 15. Anhydrous Crystalline Ruxolitinib Free Base

Dehydration of ruxolitinib di-hydrate as described in Example 14 results in the isolation of anhydrous crystalline ruxolitinib free base (Form I). Drying over P₂O₅ has resulted in the most crystalline product, and an XRPD pattern of Form I was successfully indexed using Method A for the XRPD (see FIG. 13 and Tables 9A and 9B).

TABLE 9A
Observed Peaks for Anhydrous Crystalline
Ruxolitinib Free Base (Form I)

| °2θ | d space (Å) | Intensity (%) |
|---|---|---|
| 7.27 ± 0.20 | 12.150 ± 0.334 | 100 |
| 9.51 ± 0.20 | 9.292 ± 0.195 | 29 |
| 9.71 ± 0.20 | 9.101 ± 0.187 | 12 |
| 11.07 ± 0.20 | 7.986 ± 0.144 | 9 |
| 11.49 ± 0.20 | 7.695 ± 0.133 | 60 |
| 11.61 ± 0.20 | 7.616 ± 0.131 | 53 |
| 12.38 ± 0.20 | 7.144 ± 0.115 | 26 |
| 13.15 ± 0.20 | 6.727 ± 0.102 | 86 |
| 13.96 ± 0.20 | 6.339 ± 0.090 | 40 |
| 14.59 ± 0.20 | 6.066 ± 0.083 | 13 |
| 15.36 ± 0.20 | 5.764 ± 0.075 | 54 |
| 15.65 ± 0.20 | 5.658 ± 0.072 | 43 |
| 16.05 ± 0.20 | 5.518 ± 0.068 | 31 |
| 16.62 ± 0.20 | 5.330 ± 0.064 | 12 |
| 17.19 ± 0.20 | 5.154 ± 0.060 | 15 |
| 18.15 ± 0.20 | 4.884 ± 0.053 | 91 |
| 18.50 ± 0.20 | 4.792 ± 0.051 | 31 |
| 19.11 ± 0.20 | 4.640 ± 0.048 | 52 |
| 19.58 ± 0.20 | 4.530 ± 0.046 | 69 |
| 19.93 ± 0.20 | 4.451 ± 0.044 | 17 |
| 20.47 ± 0.20 | 4.335 ± 0.042 | 12 |
| 20.96 ± 0.20 | 4.235 ± 0.040 | 20 |
| 21.55 ± 0.20 | 4.120 ± 0.038 | 22 |
| 21.95 ± 0.20 | 4.046 ± 0.036 | 36 |
| 22.26 ± 0.20 | 3.990 ± 0.035 | 15 |
| 23.10 ± 0.20 | 3.847 ± 0.033 | 15 |
| 23.94 ± 0.20 | 3.714 ± 0.031 | 82 |
| 24.72 ± 0.20 | 3.599 ± 0.029 | 12 |
| 25.52 ± 0.20 | 3.488 ± 0.027 | 14 |
| 25.62 ± 0.20 | 3.474 ± 0.027 | 15 |
| 26.25 ± 0.20 | 3.392 ± 0.025 | 32 |
| 26.59 ± 0.20 | 3.350 ± 0.025 | 18 |
| 27.32 ± 0.20 | 3.262 ± 0.023 | 11 |
| 28.16 ± 0.20 | 3.166 ± 0.022 | 13 |
| 28.34 ± 0.20 | 3.146 ± 0.022 | 11 |
| 28.86 ± 0.20 | 3.091 ± 0.021 | 11 |
| 29.46 ± 0.20 | 3.030 ± 0.020 | 18 |
| 30.20 ± 0.20 | 2.957 ± 0.019 | 7 |
| 30.91 ± 0.20 | 2.891 ± 0.018 | 8 |
| 31.39 ± 0.20 | 2.848 ± 0.018 | 12 |
| 32.13 ± 0.20 | 2.783 ± 0.017 | 5 |
| 32.77 ± 0.20 | 2.730 ± 0.016 | 6 |
| 32.93 ± 0.20 | 2.718 ± 0.016 | 6 |
| 33.61 ± 0.20 | 2.664 ± 0.015 | 7 |

TABLE 9B
Prominent Peaks for Anhydrous Crystalline
Ruxolitinib Free Base (Form I)

| | | Intensity |
|---|---|---|
| °2θ | d space (Å) | (%) |
| 7.27 ± 0.20 | 12.150 ± 0.334 | 100 |
| 9.51 ± 0.20 | 9.292 ± 0.195 | 29 |
| 11.49 ± 0.20 | 7.695 ± 0.133 | 60 |
| 11.61 ± 0.20 | 7.616 ± 0.131 | 53 |
| 12.38 ± 0.20 | 7.144 ± 0.115 | 26 |
| 13.15 ± 0.20 | 6.727 ± 0.102 | 86 |
| 13.96 ± 0.20 | 6.339 ± 0.090 | 40 |
| 15.36 ± 0.20 | 5.764 ± 0.075 | 54 |
| 15.65 ± 0.20 | 5.658 ± 0.072 | 43 |
| 16.05 ± 0.20 | 5.518 ± 0.068 | 31 |
| 18.15 ± 0.20 | 4.884 ± 0.053 | 91 |
| 18.50 ± 0.20 | 4.792 ± 0.051 | 31 |
| 19.11 ± 0.20 | 4.640 ± 0.048 | 52 |
| 19.58 ± 0.20 | 4.530 ± 0.046 | 69 |
| 21.95 ± 0.20 | 4.046 ± 0.036 | 36 |
| 23.94 ± 0.20 | 3.714 ± 0.031 | 82 |
| 26.25 ± 0.20 | 3.392 ± 0.025 | 32 |

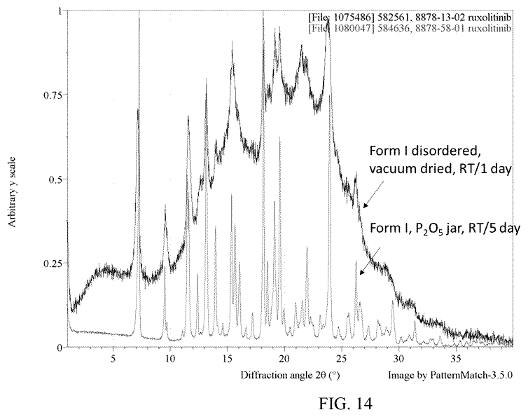

Differences in the XRPD patterns for Form I are observed with respect to degree of crystallinity. The degree of crystallinity is affected by the method of desolvation. Material exhibiting higher crystallinity is formed when the dehydration is conducted at a slower rate such as storage over P₂O₅ versus vacuum drying or heating (FIG. 14).

Two Form I samples were thermally analyzed by DSC Method B and TGA Method C. The thermal data is consistent with an anhydrous solid. TGA indicated a 0.07% weight loss up to 230° C. (FIG. 17), and the DSC depicted a single endothermic event with an onset of 83° C. (FIG. 15. The event is broad with the signal returning to baseline near 140° C. No evidence of crystallization is observed after the presumed melt at 83° C.

The second Form I sample exhibited greater weight loss in the TGA with 0.4% weight loss up to 98° C. with no appreciable additional weight loss observed until after 260° C. (FIG. 18). A single endothermic event with an onset of 81° C. is observed by DSC (FIG. 16). An exothermic trend is observed prior to the 81° C. endothermic onset and likely related to the sample increasing in Form I crystallinity prior to the melt at 81° C. The second sample of Form I was observed to contain a small quantity of diffuse scattering when compared to the first sample and would indicate the material may contain defects or minor amount of amorphous content.

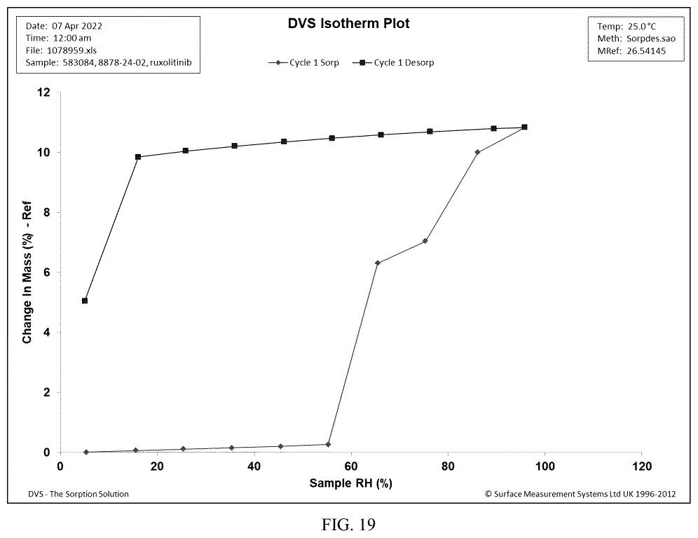

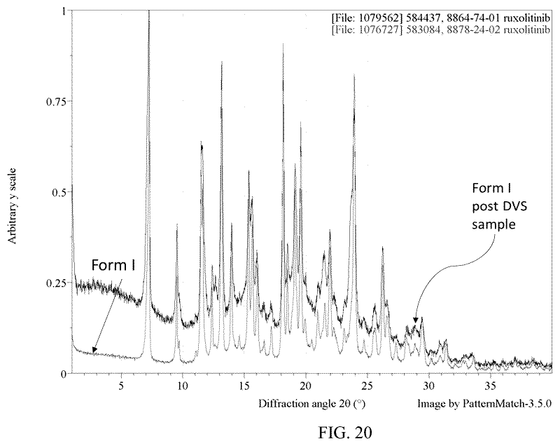

The DVS data was collected as generated via DVS Method D, and determined Form I is a hygroscopic (FIG. 19). Minimal water absorption was observed from 5 to 55% RH with a 0.26% weight gain observed. From 55 to 95% RH a 10.6% weight gain was observed (corresponding to 2 moles of water and confirmed to be the di-hydrate by variable humidity XRPD and RH stressing studies). Hysteresis is observed upon desorption. From 95 to 15% RH only 1% weight loss is observed followed by an additional 4.8% from 15 to 5% RH. The post DVS sample was observed to be Form I but was less crystalline than the starting material (FIG. 20).

TABLE 10
DVS Data

| Target | Sample | Sorp Mass | Sample | Desorp Mass | |
|---|---|---|---|---|---|
| RH | RH | Change | RH | Change | |
| (%) | (%) | (%) | (%) | (%) | Hysteresis |
| 5.0 | 5.4 | 0.00 | 5.1 | 5.04 | |
| 15.0 | 15.6 | 0.06 | 16.1 | 9.84 | 9.78 |
| 25.0 | 25.4 | 0.11 | 25.9 | 10.05 | 9.94 |
| 35.0 | 35.4 | 0.15 | 36.0 | 10.21 | 10.06 |
| 45.0 | 45.5 | 0.19 | 46.1 | 10.35 | 10.16 |
| 55.0 | 55.3 | 0.26 | 56.1 | 10.47 | 10.21 |
| 65.0 | 65.5 | 6.30 | 66.2 | 10.58 | 4.28 |
| 75.0 | 75.3 | 7.04 | 76.3 | 10.68 | 3.65 |
| 85.0 | 86.1 | 10.00 | 89.4 | 10.79 | 0.79 |
| 95.0 | 95.8 | 10.83 | 95.8 | 10.83 | |

### Example 16. Cream Formulation Produced Starting From Ruxolitinib Di-hydrate

An oil-in-water cream formulation are prepared using ruxolitinib di-hydrate at 0.5, 1.0 and 1.5% by weight of the formulation (free base equivalent). The formulation for three strengths are identical except for adjustments to the purified water quantity based on the amount of active ingredient. All excipients used in the formulation are compendial grade (i.e., USP/NF or BP) or are approved for use in topical products. The quantitative formulae for representative 400 kg batches of the cream formulation ruxolitinib di-hydrate at 0.5, 1.0 and 1.5% are also provided in Tables 11, 12, and 13, respectively.

TABLE 11

| Ingredient | Kilograms | Percentage (w/w) |
|---|---|---|
| Ruxolitinib di-hydrate | 2.24 (di-hydrate) | 0.559 (di-hydrate) |
| | 2.0 (free base) | 0.5 (free base) |
| Propylene Glycol USP | 40.0 | 10.00 |
| Methyl Paraben NF | 0.4 | 0.10 |
| Propyl Paraben NF | 0.2 | 0.05 |
| Propylene Glycol USP | 20.0 | 5.00 |
| Xanthan Gum NF | 1.6 | 0.40 |
| Light Mineral Oil NF | 16.0 | 4.00 |
| Glyceryl Stearate SE | 12.0 | 3.00 |
| Polysorbate 20 NF | 5.0 | 1.25 |
| White Petrolatum USP | 28.0 | 7.00 |
| Cetyl alcohol NF | 12.0 | 3.00 |
| Stearyl alcohol NF | 7.0 | 1.75 |
| Dimethicone 360 NF | 4.0 | 1.00 |
| Medium Chain Triglycerides NF | 20.0 | 5.00 |
| Purified Water USP (approximate) | 201 | 50.25 |
| Edetate Disodium USP | 0.2 | 0.05 |
| Polyethylene Glycol USP | 28.0 | 7.00 |
| Phenoxyethanol BP | 2.0 | 0.5 |
| Total (approximate) | 400.0 | 100 |

TABLE 12

| Ingredient | Kilograms | Percentage (w/w) |
|---|---|---|
| Ruxolitinib di-hydrate | 4.47 (di-hydrate) | 1.12 (di-hydrate) |
| | 4.0 (free base) | 1.00 (free base) |
| Propylene Glycol USP | 40.0 | 10.00 |
| Methyl Paraben NF | 0.4 | 0.10 |
| Propyl Paraben NF | 0.2 | 0.05 |
| Propylene Glycol USP | 20.0 | 5.00 |
| Xanthan Gum NF | 1.6 | 0.40 |
| Light Mineral Oil NF | 16.0 | 4.00 |
| Glyceryl Stearate SE | 12.0 | 3.00 |
| Polysorbate 20 NF | 5.0 | 1.25 |
| White Petrolatum USP | 28.0 | 7.00 |
| Cetyl alcohol NF | 12.0 | 3.00 |
| Stearyl alcohol NF | 7.0 | 1.75 |
| Dimethicone 360 NF | 4.0 | 1.00 |
| Medium Chain Triglycerides NF | 20.0 | 5.00 |
| Purified Water USP (approximate) | 198.5 | 49.6 |
| Edetate Disodium USP | 0.2 | 0.05 |
| Polyethylene Glycol USP | 28.0 | 7.00 |
| Phenoxyethanol BP | 2.0 | 0.5 |
| Total (approximate) | 400.0 | 100 |

TABLE 13

| Ingredient | Kilograms | Percentage (w/w) |
|---|---|---|
| Ruxolitinib di-hydrate | 6.71 (di-hydrate) | 1.68 (di-hydrate) |
| | 6.0 (free base) | 1.5 (free base) |
| Propylene Glycol USP | 40.0 | 10.00 |
| Methyl Paraben NF | 0.4 | 0.10 |
| Propyl Paraben NF | 0.2 | 0.05 |
| Propylene Glycol USP | 20.0 | 5.00 |
| Xanthan Gum NF | 1.6 | 0.40 |
| Light Mineral Oil NF | 16.0 | 4.00 |
| Glyceryl Stearate SE | 12.0 | 3.00 |
| Polysorbate 20 NF | 5.0 | 1.25 |
| White Petrolatum USP | 28.0 | 7.00 |
| Cetyl alcohol NF | 12.0 | 3.00 |
| Stearyl alcohol NF | 7.0 | 1.75 |
| Dimethicone 360 NF | 4.0 | 1.00 |
| Medium Chain Triglycerides NF | 20.0 | 5.00 |
| Purified Water USP (approximate) | 195.5 | 48.9 |
| Edetate Disodium USP | 0.2 | 0.05 |
| Polyethylene Glycol USP | 28.0 | 7.00 |
| Phenoxyethanol BP | 2.0 | 0.5 |
| Total (approximate) | 400.0 | 100 |

The oil-in-water cream formulations are synthesized according to the following procedure at either a 400 kg scale. Generally, overhead mixer with high and low shear mixing blades are suitable for the process.

### Procedure

1. A paraben phase is prepared by mixing methyl and propyl parabens with a portion of the propylene glycol (see % in Tables 11-13).
2. Next, a xanthan gum phase is prepared by mixing xanthan gum with propylene glycol (see % in Tables 11-13).
3. An oil phase is then prepared by mixing light mineral oil, glyceryl stearate, polysorbate 20, white petrolatum, cetyl alcohol, stearyl alcohol, dimethicone and medium chain triglycerides. The phase is heated to 70-80° C. to melt and form a uniform mixture.
4. The aqueous phase is next prepared by mixing purified water, polyethylene glycol, and disodium EDTA. The phase is heated to 70-80° C.
5. The aqueous phase of step 4, paraben phase of step 1, and ruxolitinib di-hydrate are combined to form a mixture.
6. The xanthan gum phase from step 2 is then added to the mixture from step 5.
7. The oil phase from step 3 is then combined under high shear mixing with the mixture from step 6 to form an emulsion.
8. Phenoxyethanol is then added to the emulsion from step 7. Mixing is continued, and then the product is cooled under low shear mixing.

### Example 17. Sustained-Release Dosage Form for Ruxolitinib Di-hydrate

A 25 mg sustained-release formulation of ruxolitinib di-hydrate is prepared according to the following process. The formulation components are provided in Table 14. Percentages are by weight.

TABLE 14

| Component | Function | Percentage |
|---|---|---|
| Ruxolitinib di-hydrate | Active ingredient | 10.3 (di-hydrate) |
| | | 9.2 (free base) |
| Microcrystalline cellulose, NF | Filler | 22.0 |
| Hypromellose, USP | Sustained release | 4.0 |
| (Methocel K15M) | matrix former | |
| Hypromellose, USP | Sustained release | 16.0 |
| (Methocel K4M) | matrix former | |
| Lactose monohydrate, NF | Filler | 43.6 |
| Colloidal silicon dioxide, NF | Glidant | 1.0 |
| Magnesium stearate, NF | Lubricant | 0.5 |
| Stearic acid, NF | Lubricant | 2.0 |
| Total | | 100 |

### Process

Step 1. Microcrystalline cellulose, ruxolitinib phosphate, lactose monohydrate, and hypromelloses are added to a suitable blender and mix.
Step 2. The mixture from Step 1 is transferred to a suitable granulator and is mixed.
Step 3. Purified water is added while mixing. Alternatively, the mixture is dry granulated.
Step 4. The granules from Step 3 are screened.
Step 5. The granules from Step 4 are transferred into a suitable dryer and are dried until LOD is no more than 3%.
Step 6. The granules from Step 5 are screened.
Step 7. Colloidal silicon dioxide is mixed with granules in Step 6 in a suitable blender.
Step 8. Stearic acid and magnesium stearate are mixed with the blend in Step 7 and are blended.
Step 9. The final blend in Step 8 is compressed on a suitable rotary tablet press.

Various modifications of the present disclosure, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A solid form, which is crystalline ruxolitinib di-hydrate:

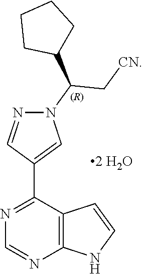

2. The solid form of claim 1, wherein the solid form is substantially isolated.

3. The solid form of claim 1, wherein the solid form is characterized by having at least one XRPD peak, in terms of 2-theta (±0.2 degrees), selected from 6.9, 10.6, 11.6, 12.9, 15.1, 15.4, 19.0, 21.8, 22.7, 23.1, 24.8, and 25.7 degrees.

4. The solid form of claim 1, wherein the solid form is characterized by having at least five XRPD peaks, in terms of 2-theta (±0.2 degrees), selected from 6.9, 10.6, 11.6, 12.9, 15.1, 15.4, 19.0, 21.8, 22.7, 23.1, 24.8, and 25.7 degrees.

5. The solid form of claim 1, wherein the solid form is characterized by having an XRPD pattern with characteristic peaks as substantially shown in FIG. 2.

6. The solid form of claim 1, wherein the solid form is characterized by single crystal x-ray diffraction having a P212121 space group and cell formula units (Z) of 8.

7. The solid form of claim 6, wherein the solid form has unit cell parameters: a is about 9.97 Å, b is about 15.18 Å, c is about 23.64 Å, α is about 90°, β is about 90°, and γ is about 90°.

8. The solid form of claim 1, wherein the solid form is characterized by having an endothermic peak with an onset temperature (±5° C.) at 61° C. and a maximum temperature (±5° C.) at 67° C., in a DSC thermogram.

9. The solid form of claim 1, wherein the solid form is characterized by having a DSC thermogram substantially as depicted in FIG. 3. The solid form of claim 1, wherein the solid form is characterized by having a TGA thermogram substantially as depicted in FIG. 4.

11. A solid form, which is anhydrous crystalline ruxolitinib free base.

12. The solid form of claim 11, wherein the solid form is substantially isolated.

13. The solid form of claim 11, wherein the solid form is characterized by having at least one XRPD peak, in terms of 2-theta (±0.2 degrees), selected from 7.2, 11.5, 11.6, 13.2, 14.0, 15.4, 18.2, 19.1, 19.6, 22.0, and 23.9 degrees.

14. The solid form of claim 11, wherein the solid form is characterized by having an XRPD pattern with characteristic peaks as substantially shown in FIG. 13.

15. The solid form of claim 11, wherein the solid form is characterized by having an endothermic peak with an onset temperature (±5° C.) at 83° C. and a maximum temperature (±5° C.) at 93° C., in a DSC thermogram.

16. The solid form of claim 11, wherein the solid form is characterized by having a DSC thermogram substantially as depicted in FIG. 15 or FIG. 16.

17. The solid form of claim 11, wherein the solid form is characterized by having a TGA thermogram substantially as depicted in FIG. 17 or FIG. 18.

18. A process of preparing a solid form of claim 1, which is ruxolitinib di-hydrate: 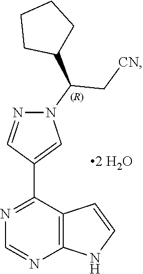
comprising isolating the solid form from a solution comprising ruxolitinib free base and an aqueous solvent component.

19. The process of claim 18, wherein the aqueous solvent component comprises a polar protic solvent and water, wherein the polar protic solvent is isopropanol.

20. The process of claim 18, wherein the ruxolitinib free base is prepared by a process comprising reacting ruxolitinib phosphate: 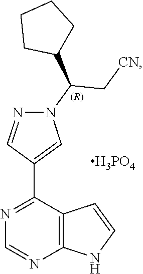
with a base in a solvent component, wherein:
the reacting of ruxolitinib phosphate with a base comprises using from about 2 to about 3 molar equivalents of the base relative to ruxolitinib phosphate.
the base is a hydroxide base; and
the solvent component comprises water, an ester solvent, a halogenated solvent, or a mixture thereof, wherein the ester solvent is ethyl acetate; and wherein the halogenated solvent is dichloromethane.

21. A process of preparing a solid form of claim 11, comprising drying crystalline ruxolitinib di-hydrate.

22. The process of claim 21, wherein the drying comprising drying crystalline ruxolitinib di-hydrate in ajar with desiccant at about room temperature.

23. A pharmaceutical composition comprising the solid form of claim 1.

24. A pharmaceutical composition comprising the solid form of claim 11.

25. The pharmaceutical composition of claim 23, which is an oral dosage form.

26. The pharmaceutical composition of claim 25, wherein the oral dosage form is an immediate dosage form.

27. The pharmaceutical composition of claim 26, wherein the solid form is ruxolitinib di-hydrate, which is present in an amount of about 5 to about 25 mg on a free base basis.

28. The pharmaceutical composition of claim 25, wherein the oral dosage form is a sustained-release dosage form.

29. The pharmaceutical composition of claim 28, wherein the solid form is ruxolitinib di-hydrate, which is present in an amount of about 10 to about 50 mg on a free base basis.

30. The pharmaceutical composition of claim 23, wherein the composition is a topical formulation.

31. The pharmaceutical composition of claim 30, wherein the topical formulation is a cream formulation.

32. The pharmaceutical composition of claim 31, wherein the cream formulation comprises an oil-in-water emulsion.

33. The pharmaceutical composition of claim 32, wherein the cream formulation is prepared by incorporating ruxolitinib di-hydrate in the oil-in-water emulsion.

34. A topical pharmaceutical formulation, comprising (a) ruxolitinib, or a pharmaceutically acceptable salt thereof, and (b) a solid form of claim 1, which is present in an amount of less than about 0.9%, less than about 0.8%, less than about 0.7%, less than about 0.6%, less than about less than about 0.4%, less than about 0.3%, less than about 0.2%, less than about 0.1%, less than about 0.09%, less than about 0.08%, less than about 0.07%, less than about 0.06%, less than about 0.05%, less than about 0.04%, less than about 0.03%, less than about 0.02%, less than about 0.01%, less than about 0.009%, less than about 0.008%, less than about 0.007%, less than about 0.006%, less than about 0.005%, less than about 0.004%, less than about 0.003%, less than about 0.002%, or less than about 0.001% on a free base basis by weight of the formulation.

35. The topical pharmaceutical formulation of claim 34, wherein the topical pharmaceutical formulation is prepared at a large batch size.

36. The topical pharmaceutical formulation of claim 34, wherein the ruxolitinib, or the pharmaceutically acceptable salt thereof, is present in an amount of about 0.5% to about 1.5% on a free base basis by weight of the formulation.

37. The topical pharmaceutical formulation of claim 34, wherein the ruxolitinib, or the pharmaceutically acceptable salt thereof, is present in an amount of about 1.5% on a free base basis by weight of formulation.

38. The topical pharmaceutical formulation of claim 37, wherein the ruxolitinib, or the pharmaceutically acceptable salt thereof, is ruxolitinib phosphate.

39. A process for releasing a batch of the topical pharmaceutical formulation of claim 34, comprising (i) testing a sample of the topical pharmaceutical formulation for the absence of crystalline ruxolitinib di-hydrate; and, if the sample passes the test in step (i), then: (ii) releasing the batch for public use.

40. The process of claim 39, wherein the testing comprises observing a sample of the formulation under a light microscope in order to detect the absence or presence of crystals, wherein the sample passes the test when crystals are not detected.

41. A method of treating a disease in a patient in need thereof, comprising administering to the patient a pharmaceutical composition of claim 23, wherein the disease is myelofibrosis, polycythemia vera, acute graft versus host disease or chronic graft versus host disease.

42. A method of treating a skin disorder in a human patient in need thereof, comprising administering to the patient a pharmaceutical composition of claim 23.

43. A method of treating a skin disorder in a human patient in need thereof, comprising administering to an affected skin area of the patient a topical pharmaceutical formulation of claim 34.

44. The method of claim 42, wherein the skin disorder is an autoimmune skin disease.

45. The method of claim 44, wherein the skin disorder is atopic dermatitis, lichen planus, hidradenitis suppurativa, psoriasis, skin rash, skin irritation, skin sensitization, contact dermatitis or allergic contact dermatitis, or bullous pemphigoid.

46. A method of treating a disease in a patient in need thereof, comprising administering to the patient a pharmaceutical composition of claim 24, wherein the disease is myelofibrosis, polycythemia vera, acute graft versus host disease or chronic graft versus host disease.

47. A method of treating a skin disorder in a human patient in need thereof, comprising administering to the patient a pharmaceutical composition of claim 24.
